(19) United States
(12) Patent Application Publication
(10) Pub. No.: US 2025/0034591 A1
(43) Pub. Date: Jan. 30, 2025

(54) SELF-INACTIVATING LENTIVIRAL VECTOR ENCODING BETA- OR GAMMA-GLOBIN

(71) Applicant: Children's Hospital Medical Center, Cincinnati, OH (US)

(72) Inventor: Punam Malik, Cincinnati, OH (US)

(21) Appl. No.: 18/737,201

(22) Filed: Jun. 7, 2024

Related U.S. Application Data
(63) Continuation of application No. 16/197,164, filed on Nov. 20, 2018, now abandoned.
Application No. 16/197,164 is a continuation of application No. 14/707,557, filed on May 8, 2015, now abandoned.
Application No. 14/707,557 is a continuation of application No. 12/928,302, filed on Dec. 6, 2010, now abandoned.
(60) Provisional application No. 61/267,008, filed on Dec. 4, 2009.

(51) Int. Cl. C12N 15/86; C12N 15/63; C12N 15/85

(52) U.S. Cl. CPC C12N 15/86; C12N 15/63; C12N 15/85; C12N 2740/15043; C12N 2830/48

(57) ABSTRACT

Methods and compositions disclosed herein generally relates to methods of determining minimum hematopoietic stem cell (HSC) chimerism and gene dosage for correction of a hematopoietic disease; in particular, in in vivo models. The invention also relates to modified lentiviral expression vectors for increase a viral titer and various methods for increasing such titers as well as expression vectors capable of enhancing such titers. The invention also relates to CHS4 chromatin insulator-derived functional insulator sequences. The invention further relates to methods for genetic correction of diseases or reducing symptoms thereof, such as sickle cell anemia, a lysosomal storage disease. The invention further relates to a method of improving and/or correcting one or more central nervous system (CNS) abnormalities caused by one or more lysosomal storage disease. The invention further relates to methods of improving titer in transfection-based bioreactor culture production or transfection-based production systems using eukaryotic cells.

### CROSS REFERENCE TO RELATED APPLICATIONS

This application is a continuation of U.S. application Ser. No. 16/197,164, filed on Nov. 20, 2018, which is a continuation of U.S. application Ser. No. 14/707,447 filed on Dec. 6, 2010, which is a continuation of U.S. Ser. No. 12/928,302 filed on Dec. 6, 2010, which claims priority to U.S. Provisional Application No. 61/267,008, filed on Dec. 4, 2009, the contents of each of which are herein incorporated by reference in their entirety.

### GOVERNMENT RIGHTS

This invention was made with government support under HL073104, HL070135, and HL070595, awarded by the National Institutes of Health. The government has certain rights in the invention.

### FIELD OF THE INVENTION

The invention disclosed herein generally relates to methods of determining minimum hematopoietic stem cell (HSC) chimerism and gene dosage for correction of a hematopoietic disease; in particular, in an in vivo model. The invention also relates to the use of modified SIN lentiviral expression vectors to increase a viral titer and various methods for increasing such titers as well as expression vectors capable of enhancing such titers. The invention also relates to CHS4 chromatin insulator-derived functional insulator sequences. The invention further relates to methods for genetic correction of diseases or reducing symptoms thereof, such as sickle cell anemia, a lysosomal storage disease. The invention further relates to various expression vectors capable of genetically correcting sickle cell anemia or reducing symptoms thereof. The invention further relates to a method of improving and/or correcting one or more central nervous system (CNS) abnormalities caused by one or more lysosomal storage disease. The invention further relates to methods of improving titer in transfection-based bioreactor culture production or transfection-based production systems using a eukaryotic cell.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Genetic Correction and Vector Design

Successful genetic correction of diseases, mediated by hematopoietic stem cells (HSCs), depends upon stable, safe, targeted gene expression of therapeutic quantities. Expression vectors are central to the process of genetic correction and consequently the subject of considerable research. Although significant advances in vector design have improved the efficacy of gene therapy, certain key obstacles have emerged as barriers to successful clinical application. Among those obstacles, vector genotoxicity is among the most formidable, as evidenced by the occurrence of gene therapy related leukemia in patients in X-SCID trials, as disclosed herein. As a result, gamma-retroviral vectors and lentiviral vectors have been modified to a self-inactivating (SIN) design to delete ubiquitously active enhancers in the U3 region of the long terminal repeats (LTR) (as disclosed herein). Several methods of improving transgene expression have been subsequently employed.

As an added measure of stabilizing expression, many vectors are now designed with chromatin insulating elements that reduce chromatin position effects. While these insulators can improve the safety and expression profiles of certain vectors, in some cases an undesirable side effect is decreased titers compared to non-inactivated versions.

Thus, there is a need in the art for improved expression vector design, aimed at safely stabilizing the expression of transgenes, while maintaining clinically relevant viral titers.

### Reprogramming Erythroid Cells

Healthy individuals can produce 2.4×10¹¹ RBC per day with a daily output of 7.2 g of hemoglobin. Redirecting a portion of the formidable protein synthesis machinery in maturing erythroid cells toward the expression of a transgene can provide an efficient approach for long-term protein delivery into circulation. Moreover, the high efficiency of protein synthesis can compensate for the generally low hematopoietic (HSC) gene transfer frequency. Finally, expulsion of the nucleus by erythroid cells upon maturation to reticulocytes is arguably one of the most radical and effective safety measures imaginable. Thus, there is a need in the art to determine methods of using erythroid cells as a depot to deliver corrective proteins.

### Treatment of Lysosomal Storage Diseases

Lysosomal storage disorders (LSD) include about 50 metabolic diseases that collectively affect approximately 1 in 5000 live births with ˜65% affecting the CNS. Treatment modalities for LSDs are currently limited to bone marrow transplantation (BMT) and enzyme replacement therapy (ERT). These approaches, while providing significant promise for treatment of the visceral manifestations of LSDs, do little to address CNS pathologies for this group of disorders. Moreover, BMT is limited by procedure-related mortality between 20 and 30%, late complications such as graft versus host disease, and by the need to find an HLA-matched donor. Pharmaceutical lysosomal enzyme products are available for several LSDs and are being used to ameliorate visceral manifestations in some LSD patients. However, it is limited by poor penetration of the CNS, the need for frequent intravenous infusion for a lifetime and by tremendous costs. Thus, there is a need in the art to develop a novel therapeutic approach for treatment of LSDs with lower mortality and morbidity, and with the capacity to correct CNS deterioration.

### Determining Critical Parameters of Correction in Sickle Cell Anemia

Expressing a tremendous amount of fetal/antisickling hemoglobin will undoubtedly correct disease, as has been demonstrated, but is not practically possible in a clinical setting. As an example, an initial gene therapy for adenosine deaminase (ADA) deficiency was performed using no conditioning, and was not therapeutic, even though few gene-marked stem cells engrafted, and a selective advantage to gene-corrected lymphocytes was evident upon withdrawal of ADA (as disclosed herein). In a subsequent trial, 4 mg/kg busulfan was used before transplantation, as conditioning, resulting in adequate gene-corrected stem cell dose and gene-modified T cells (as disclosed herein). Thus, there is a need in the art to establish methods of determining thresholds for genetic correction before embarking on clinical studies.

### Improvement of Viral Titer

Significant research has been devoted to improving viral titer by manipulating the parameters of production in closed system bioreactors. Increases in titer translate into practical benefits, including decreased costs and the related potential for expanding the patient base for clinical trials. Thus, there is a continued need in the art for improving titer by optimizing the parameters of bioreactor vector production.

### SUMMARY OF THE INVENTION

Methods and composition described herein are provided by way of example and should not in any way limit the scope of the invention.

In one aspect, a method of determining minimum hematopoietic stem cell (HSC) chimerism and gene dosage for correction of a hematopoietic disease in an in vivo model is provided. The method comprises: inducing various levels of chimerism and gene dosage post-transplantation, wherein various levels of chimerism and gene dosage are induced by applying reduced intensity conditioning prior to transplantation; transducing cells at a range of multiplicity of infection (MOI), where MOI is 30-100; and inducing varying levels of chimerism and gene dosage results in determining minimum HSC chimerism and gene dosage for correction of a hematopoietic disease in an in vivo model.

In another aspect, a modified SIN lentiviral expression vector is provided, which is capable of enhanced viral titer in comparison with a standard SIN lentiviral expression vector. More specifically, the vector comprises: a gutted/minimal Cis lentiviral vector backbone devoid of Cis elements, except the packaging signal (w); and a small therapeutic transgene of interest (GOI), where the modified SIN lentiviral expression vector containing devoid of Cis elements, except a ψ Cis element, enhances viral titer in comparison with a standard SIN lentiviral expression vector.

In another aspect, a cHS4 chromatin insulator-derived functional insulator sequence is provided. The functional insulator sequence comprises: a proximal core region of the cHS4 insulator sequence comprising 250 base pairs (bp) or less; and a distal element of the cHS4 insulator sequence, comprising 400 bp or less, where the functional insulator sequence is at least 80% as effective as an unmodified cHS4 chromatin insulator, and where the functional insulator sequence permits a higher titer of expression of a vector carrying such a sequence, in comparison with a vector carrying the unmodified cHS4 chromatin insulator.

In another aspect, a method of increasing a titer of a modified SIN lentiviral vector, when compared to a standard SIN lentiviral vector, is provided. The method comprises: inserting one or more copies of a heterologous polyadenylation (polyA) signal sequence downstream from a viral 3′ long terminal repeat sequence in a standard SIN lentiviral vector backbone, resulting in a poly A-modified SIN lentiviral vector; and transfecting a eukaryotic cell with the poly A-modified vector, where insertion of the heterologous polyA signal sequence and subsequent transfection of the eukaryotic cell increases the viral titer of the modified vector, when compared to the standard SIN lentiviral vector. In some embodiments, the insertion of the heterologous polyA signal sequence and subsequent transfection of the eukaryotic cell reduces transcriptional read-through of vector transcripts, improves packaging efficiency and increases the viral titer of the modified vector, when compared to the standard SIN lentiviral vector.

In another aspect, a method of increasing titer of a modified SIN lentiviral vector, when compared to a standard SIN lentiviral vector, is provided. The method comprises: inserting one or more of an upstream polyA enhancer signal into a 3′ LTR of a standard SIN lentiviral vector backbone, resulting in a polyA enhancer-modified lentiviral vector; and transfecting a eukaryotic cell with the polyA enhancer-modified lentiviral vector, where transfection with the polyA enhancer-modified vector results in an increased titer as compared to titer of a standard SIN lentiviral vector. In some embodiments, the polyA enhancer is a USE sequence derived from an SV40 late polyA signal. In some embodiments, transfection with the USE sequence derived vector increases titer by reducing transcriptional read-through of vector transcripts and improving packaging efficiency. In some embodiments, three upstream polyA enhancer signal sequences are inserted into the 3′ LTR of a standard SIN lentiviral vector backbone.

In another aspect, a method of increasing titer of a multiply modified SIN lentiviral vector, when compared to a standard SIN lentiviral vector is provided. The method comprises: providing a standard SIN lentiviral vector, wherein a backbone of the vector comprises a viral 3′ long terminal repeat (LTR) sequence and a U3 deletion; obtaining a multiply modified SIN lentiviral vector and transfecting a eukaryotic cell with the multiply modified vector, where the multiply modified vector permits an increase of viral titer, compared to a standard SIN lentiviral vector. In some embodiments, the multiply modified SIN lentiviral vector is created, for example, by inserting, in any order: a heterologous polyA signal sequence downstream from the viral 3′ LTR sequence; and an upstream sequence derived from an SV40 late polyA signal into the U3 deletion.

In another aspect, an expression vector capable of enhanced titer in comparison with a corresponding lentiviral vector is provided. The expression vector comprises at least one of: a β-growth hormone polyA signal sequence downstream from a viral 3′ LTR sequence in a standard SIN lentiviral vector backbone; and a USE sequence derived from an SV40 late polyA signal, inserted into a U3 deletion region of a standard SIN lentiviral vector backbone.

In another aspect, an expression vector capable of enhancing stability and safety of gene expression while maintaining a clinically useful titer of a lentiviral vector is provided. The expression vector comprises: a heterologous polyA signal sequence downstream from a viral 3′ LTR sequence in a standard SIN lentiviral vector backbone; a USE sequence derived from an SV40 late poly A signal in a U3 deletion region of a standard SIN lentiviral vector backbone; one or more flanking cHS4-derived reduced-length functional insulator sequences; and a lineage-specific promoter and/or enhancer selected for restricted activation in cells in which expression is desired.

In another aspect, an expression vector capable of enhancing stability and safety of transgene expression while maintaining a clinically useful titer of a lentiviral vector is provided. The expression vector comprises: a transgene of interest; a heterologous poly A signal sequence downstream from a viral 3′ LTR sequence in a standard SIN lentiviral vector backbone; a USE sequence derived from an SV40 late polyA signal in a U3 deletion region of a standard SIN lentiviral vector backbone; one or more flanking cHS4-derived reduced-length functional insulator sequences; a lineage-specific promoter and/or enhancer selected for restricted activation in cells in which expression is desired; and one or more lentivirus non-coding cis sequences, selected from: R, U5, a packaging signal, rev response element, env splice acceptor site, and an extended gag sequence.

In another aspect, a method for genetic correction of sickle cell anemia or reducing symptoms thereof vector is provided. The method comprises: treating a subject with reduced intensity conditioning; and transplanting autologous hematopoietic stem cells (HSCs) transfected with a modified lentivirus where the modified lentivirus comprises: a gamma-globin gene and beta-globin locus control region; wherein: post-transplantation fetal hemoglobin exceeds at least 20%; F cells constitute at least ⅔ of the circulating red blood cells; fetal hemoglobin per F cells account for at least ⅓ of total hemoglobin in sickle red blood cells; and at least 20% gene-modified HSCs re-populate bone marrow of the subject.

In another aspect, a human gamma-globin lentiviral expression vector capable of genetically correcting sickle cell anemia or reducing symptoms thereof is provided, The expression vector comprises: a gamma-globin gene cloned in reverse orientation to a viral transcriptional unit; one or more elements of a beta-globin locus control region cloned in reverse orientation to the viral transcriptional unit; a heterologous polyA signal sequence downstream from a viral 3′ LTR sequence in a standard SIN lentiviral vector backbone; one or more USE sequences derived from an SV40 late polyA signal in a U3 deletion region of a standard SIN lentiviral vector backbone; and one or more flanking CHS4-derived reduced-length functional insulator sequences.

In another aspect, a human gamma-globin lentiviral expression vector capable of genetically correcting sickle cell anemia or reducing symptoms thereof is provided, The expression vector comprises: a gamma-globin gene cloned in reverse orientation to a viral transcriptional unit; an erythroid lineage specific enhancer element; a heterologous poly A signal sequence downstream from a viral 3′ LTR sequence in a standard SIN lentiviral vector backbone; a USE sequence derived from an SV40 late polyA signal in a U3 deletion region of a standard SIN lentiviral vector backbone; and one or more flanking CHS4-derived reduced-length functional insulator sequences.

In another aspect, a lentiviral expression vector capable of genetically correcting beta-thalassemia or reducing symptoms thereof is provided. The expression vector comprises: a beta-globin gene cloned in reverse orientation to the viral transcript; one or more elements of a beta-globin locus control region cloned in reverse orientation to the viral transcriptional unit; a heterologous polyA signal sequence downstream from a viral 3′ LTR sequence in a standard SIN lentiviral vector backbone; a USE sequence derived from an SV40 late polyA signal in a U3 deletion region of a standard SIN lentiviral vector backbone; and one or more flanking cHS4-derived reduced-length functional insulator sequences.

In another aspect, a method of genetically modifying an erythroid cell for utilization as a depot to produce and expel proteins non-native to blood and/or not conventionally secreted into blood circulation is provided. The method comprises: transducing an HSC with a vector, the vector comprising: an erythroid specific promoter; a gene of interest (GOI) operably linked to the promoter, the GOI encoding a protein non-native to blood and/or not conventionally secreted into blood circulation, where activation of the erythroid specific promoter leads to expression and expulsion by an erythroid cell of a protein or proteins non-native to blood and/or not conventionally secreted into blood circulation.

In another aspect, a method of genetically correcting Mucopolysaccharidosis type I (MPS I) and/or reducing symptoms thereof is provided. The method comprises: transducing a HSC with a vector, which vector comprises: an erythroid specific promoter; and a gene encoding alpha-L-iduronidase (IDUA), wherein activation of the erythroid specific promoter leads to the expression and expulsion of IDUA by an erythroid cell; and introducing the HSC into an individual with MPS I, where the expression and explusion of IDUA from erythroid offspring of genetically modified HSC leads to high IDUA levels in blood stream, and results in a correction of MPS I or a reduction of symptoms thereof.

In another aspect, an expression vector capable of genetically correcting MPS type I and/or reducing symptoms thereof is provided. The expression vector comprises: an erythroid specific promoter; and a gene encoding IDUA, wherein activation of the erythroid specific promoter leads to sustained expression and expulsion of IDUA by developing erythroid cells and the genetic correction of MPS I and/or reduction of symptoms thereof.

In another aspect, an expression vector capable of genetically correcting or reducing symptoms of a disease is provided. The expression vector is characterized by insufficient expression of a least one functional protein non-native to blood and/or not conventionally secreted and comprises: an erythroid specific promoter; and a GOI encoding a protein non-native to blood and/or not conventionally secreted, where activation of the erythroid specific promoter leads to the expression and expulsion by an erythroid cell of a protein or proteins non-native to erythroid cells and/or not conventionally secreted by cells.

In another aspect, a method of genetically correcting a lysosomal storage disease is provided. The method is characterized by deficiency of one or more lysosomal enzymes that can be imported into a cell through mannose 6-phosphate receptor mediated endocytosis and comprises: transducing an HSC with a vector and introducing the HSC into an individual with a lysosomal storage disease, wherein the expression and expulsion of a lysosomal enzyme from erythroid offspring of genetically modified HSCs leads to normal or high sustained lysosomal enzyme levels in an individual's blood stream; and the enzyme is endocytosed into cells through mannose 6-phosphate mediated endocytosis, resulting in correction of a lysosomal storage disease and/or a reduction of symptoms thereof. The vector comprises: an erythroid specific promoter; and a gene encoding a lysosomal enzyme that can be released when over-expressed in a cell, where activation of the erythroid specific promoter leads to the expression and expulsion of a lysosomal enzyme by an erythroid cell.

In another aspect, a method of improving and/or correcting one or more central nervous system (CNS) abnormalities caused by one or more lysosomal storage disease is provided. The method comprises: transducing an HSC with a vector, and introducing the HSC into an individual with a lysosomal storage disease, where the expression and expulsion of a lysosomal enzyme from erythroid offspring of genetically modified HSCs leads to sustained normal or above normal lysosomal enzyme levels in an individual's blood stream, and correction or improvement of one or more CNS abnormalities caused by one or more lysosomal storage diseases. The vector comprises: an erythroid specific promoter; and a gene encoding a lysosomal enzyme that can be released when over-expressed in a cell, wherein activation of the erythroid specific promoter leads to the expression and expulsion of a lysosomal enzyme by an erythroid cell.

In another aspect, a method of improving viral titer in transfection-based production system using a eukaryotic cell is provided. The method comprises at least one of: harvesting a population of eukaryotic cells prior to transfection that have progressed beyond log phase of cell growth to a state of confluency for at least 24 hours; mixing the population with transfection reagents and plasmid DNA at the time of re-seeding into a new culture vessel, where the harvesting and mixing steps, alone or in combination, results in an improved viral titer, by at least 2-fold, in a transfection-based production using a eukaryotic cell. In another aspect, a method of improving titer in transfection-based production using a eukaryotic cell is provided. The method comprises at least one of: harvesting of a confluent population of eukaryotic cells for transfection that have progressed beyond log phase of growth; mixing the population with transfection reagents and plasmid DNA at the time of seeding; and seeding cells at a cell density of at least 5×10⁴ cells/cm² 4 to 5 days prior to cell harvest and transfection, where any of the harvesting, mixing, and/or seeding, alone or in any combination, results in an improved titer, by at least 2-fold, in a transfection-based production using a eukaryotic cell.

In another aspect, a method of improving titer in transfection-based bioreactor culture production using a eukaryotic cell is provided. The method comprises at least one of: harvesting of a confluent population of eukaryotic cells for transfection that have progressed beyond log phase of growth; mixing the population with transfection reagents and plasmid DNA at the time of seeding; seeding cells at a cell density of at least 5×10⁴ cells/cm² 4 to 5 days prior to cell harvest and transfection; and transfecting of cells with at least 9.2 μg/ml of plasmid DNA, using either suspension cells or cells to be plated onto carriers or microcarriers, wherein any of the harvesting, mixing, seeding, and/or transfecting steps, alone or in any combination, results in an improved titer, by at least 2-fold, in transfection-based bioreactor culture production using a eukaryotic cell.

### BRIEF DESCRIPTION OF THE FIGURES

Those of skill in the art will understand that the drawings, described below, are for illustrative purposes only. The drawings are not intended to limit the scope of the present teachings in any way.

FIGS. 1A and 1B depict titers from the standard and gutted SIN-LV (a) A schematic representation of SIN lenti-proviruses. sSIN-GFP, sBG-6 and sFIG are SIN-LV carrying GFP, BG or the Fanconi Anemia A cDNA, -IRES-GFP respectively. dsSIN-GFP, sBG-1 and ds-FIG are their gutted counterparts. SD=splice donor. SA=splice acceptor. ψ packaging sequence. cPPT: central poly purine tract. The gag (360 bp) and the env fragment containing the RRE (-850 bp) are indicated. (b) The viral obtained after infection of MEL cells and analysis for GFP and hβ-globin expressing cells. Titers are expressed as IU/mL of concentrated supernatant (n=3).

FIG. 2 depicts BG SIN-LV constructs. A schematic representation of 10 SIN-lentiviral proviral forms (sBG-1 to sBG-10). All the vectors contain BG (HS2, 3, and 4 elements of the LCR, the (β-promoter and gene) and cPPT. Gag (630 bp or 360 bp), RRE, env fragments are shown. * indicates a point mutation that disrupts the SA.

FIGS. 3A and 3B depict viral titers of BG SIN-LV (a) Viral supernatants of sBG-1 to sBG-10 SIN lentiviral vectors were concentrated 1400-fold and titered on MEL cells by monitoring for β-globin positive cells by flow cytometry (n=4). (b) Fold increase in titers with inclusion of cis-elements. The titers were normalized to that of the completely gutted vector (sBG-1), which was considered 1.

FIGS. 4A and 4B depict effect of LV cis-elements on the provirus stability and expression. (a) Proviral integrity: Southern blot analysis of MEL cells transduced with sBG-127 to sBG-10, restricted with AflII that cuts in the viral LTRs, and probed with a hβ-globin fragment. (b) Expression of hβ-globin in MEL cells: dot plot analysis of sBG-1 to sBG-10 transduced MEL cells from one representative experiment; MFI are indicated in the upper right corner of the dot-plot.

FIGS. 5A and 5B depict vRNA transcripts in packaging cells. Northern blot analysis of (A) total RNA from 293T packaging cells transfected with SIN LV plasmids and probed with a 32P labeled hp-globin fragment. Lower panel shows the same blot hybridized with an 18S probe as loading control. A full length band of the expected size is visible for all the vectors. * indicate vectors in which SA is present and both full length and spliced bands are visible. A small schematic of the vector cis-sequences are shown above the vector lanes to depict the & packaging sequence; R: RRE; SA: Splice Acceptor in the env fragment; SG: short gag fragment (360 bp); LG: long gag fragment (630 bp) in vectors. (B) Cytoplasmic RNA for vectors with and without RRE from the same experiment shown in panel A, showing the efficiency of vRNA export into the cytoplasm. The phosphoimager quantified ratios cytoplasmic/total are shown in FIG. 6E.

FIGS. 6A through 6E depict packaging of vRNA into virons (a) A representative dot blot analysis on vRNA extracted from sBG series of virus supernatants showing that the amount of vRNA is proportional to infectious titers. Virus was made from all ten vectors and concentrated identically as described and the dot-blot was probed with a β-globin fragment. NC-negative control. Four different dilutions for each vector were loaded in duplicate in the representative experiment shown. A total of three experiments were performed (B) Phosphoimager counts obtained on the 28 dot blot shown in panel (A). (C) Relative quantification of vRNA from all three experiments. (D) p24 activity in concentrated virus from all vectors (n=2). (E) Ratio of Cytoplasmic/Total RNA from 2 Northern Blot Analysis (NB) in Packaging Cells (The ratio cytoplasmic/total RNA was normalized to the value for the completely gutted vector lacking the RRE (SBG-1) and to 18S RNA (for loading) in two independent experiments. Analogous vectors with and without RRE are marked as I, II and III to allow ready comparisons).

FIGS. 7A and 7B depict vector constructs and experimental design. A. Self-inactivating (SIN) lentiviral vector carrying the hβ-globin gene and the HS2, HS3 and HS4 of the locus control region is shown as sBG. Using this backbone, a series of vectors were generated to incorporate either the cHS4 59 250 bp core, 2 tandem repeats of the core, 5′ 400 bp or 59 800 bp of cHS4, and the full-length 1.2 Kb cHS4 insulator. Vectors sBG400S and sBG800S carry in addition to the core inert DNA spacers from 1 bacteriophage. B. Schema of In vitro and in vivo analyses: MEL cells were transduced with various vectors to derive single copy MEL clones and hβ-globin expression and ChIP analysis was performed in differentiated clones. In vivo analysis was done using vector transduced Hbb^{th3±} donor LSK cells transplanted into lethally irradiated Hbb^{th3/±} recipients and analyzed at 6 months post-transplant. Secondary transplants were performed for CFU-S analysis. C. Representative FACS plot showing hβ-globin-expressing cells (% hβ+) for uninsulated (sBG, green) and insulated (sBG-I, Pink) single copy MEL clone with coefficient of variation (CV) of expression shown by arrows.

FIGS. 8A and 8B depict human β-globin expressing cells in MEL clones. A. Proportion of hβ-globin-expressing cells (% hβ+) in MEL clones. Each circle represents an individual single copy MEL clone. B. CV values of hβ-globin expression of each clone. The means are represented with a horizontal line and the mean 6 SEM of % hβ+ MEL cells and CV of hβ-globin expression for each vector are indicated in the box above. Filled circles represent representative clones picked for ChIP analysis. *P<0.05 by ANOVA, as compared to sBG.

FIGS. 9A through 9E depict human β-globin expression in RBCs and single copy secondary CFU-S. A. Representative FACS histograms showing (% hβ+ RBC are indicated within the histogram). B. Cumulative data on the percentage of hβ+ RBCs normalized to vector copy. C. The coefficient of variation (CV) of hβ expression in RBCs. D. Cumulative data on % hβ+ cells/CFU-S. Each circle represents an individual single integrant CFU-S. E. The CV of hβ expression in the individual CFU-S. Numbers above bar diagrams represent mean 6 SEM and values significantly different from controls by ANOVA are marked by an asterisk. *P<0.05; * P<0.01.

FIGS. 10A through 10F depict chIP analysis showing the active and repressive histone marks on the 5′ 250 bp cHS4 core and the hβ promoter in MEL cell clones. A. Map of the proviral form of the vector. Arrows show the position of the primer pairs used for PCR and qPCR; and the lines represent insulator fragments. B-C. ChIP with antibodies against control IgG, acH3, acH4, H3K4-me2, H3K9-me3 and H3K27-me3 and semiquantitative PCR primers to the β-globin promoter region D-F ChIP with antibodies to AcH3 and AcH4 (D), H3K4-me2 (E); H3K9-me3 and H3K27-me3 (F) followed by qPCR using primers amplifying cHS4 core (left panels) and hβ-globin promoter (right panel) on pooled clones (shown in FIG. 2). *P<0.05; **P<0.01.

FIGS. 11A and 11B depict human β-globin expression in mice. A. RBC parameters, reticulocytes and vector copies. Values represent means±SEM. Hb=hemoglobin, MCV=mean corpuscular volume, MCHC=mean corpuscular hemoglobin concentration, vector copy=vector copies in leukocytes by qPCR. B. HPLC analysis of human β-globin protein in blood lysates as a percentage of total hemoglobin [hβ-ma/(hβ-ma+mβ-ma)]. Data is normalized to vector copy/cell in leukocytes. *P<0.05; **P<0.01.

FIGS. 12A through 12G depict effect of 3′400 bp region of the cHS4 insulator. A. Vector design of sBG^{3′400} vector. The full length cHS4 is shown for comparison. B-C. Proportion of hβ+ cells (B) and the coefficient of variation of hβ expression of sBG^{3′400} (C) in MEL clones. Each circle represents a single integrant MEL clone. The means are represented with a horizontal line and the mean±SEM are represented in the figure. D-E. The percentage of hβ-globin+ RBC (D), and the CV of hβ expression (E) in mice. F-G. hβ-globin-expressing cells (F) and the CV of hb expression (G) in single copy CFU-S following secondary transplant. Each circle represents individual CFU-S. Mean±SEM and P-values are shown. *P<0.05; **P<0.01; ***P<0.001.

FIGS. 13A through 13G depict effect of the combination of the 5′ core with the 3′ 400 bp regions of the cHS4 insulator. A. Vector design of sBG650. The full length cHS4 is shown for comparison. B. Proportion of hβ+ cells and C. CV of hβ-globin expression in sBG65° MEL clones. Each circle represents a single copy MEL clone. The means are represented with a horizontal line and the mean±SEM is indicated above each group. D. Percentage of hbglobin expressing RBC in ransplanted mice. E. Percentage hβ-globin expressing cells in single copy CFU-S from secondary mice. F-G. ChIP active and repressive chromatin followed by semiquantitative PCR (F) or qPCR (G) of the cHS4 core region or the hβ-globin promoter region.

FIGS. 14A through 14J depict chromatin patterns over the 3′400 bp and its interaction with the 5′ core region. A. A map of 3′LTR showing location of full length 1.2 kb insulator and the position of primers used in ChIP analysis. Vectors tested with the indicated regions of the cores are depicted beneath map B ChIP with antibodies to AcH3 and AcH4, H3K4-me2 and H3K9-me3 and H3K27-me3 followed by a semiquantitative PCR of the 3′400 region in sBG^{3′400}, sBG⁶⁵⁰, sBG-I provirus. C-D ChIP with antibodies to USF-1 and CTCF followed by semi-quantitative PCR (C) or qPCR (D) for the core region. E-F ChIP with antibodies to USF-1 and CTCF followed by semi-quantitative PCR (C) or qPCR (D) for the 3/400 bp region of the sBGC, sBG^{3′400}, sBG⁶⁵⁰ and sBG-I provirus in pools of three single copy MEL clones. (G) Representative histograms (FACS) showing hb expressing cells in mock, sBG, sBGC, sBG2C, sBG400 and sBGI sBGI single copy CFU-S. The % of hβ+ cells are indicated within the histogram. (H) Human β-globin messenger RNA (mRNA) expressionin single copy secondary CFU-S of sBG, sBGC, sBG2C, sBG400 and sBG-I by qPCR. Murine a-globin expression servedas the internal control against which hβ-globin expression was normalized. P values are shown in the figure. ** indicates P<0.01. (I) The primers and probes (SEQ ID NOs: 19-30) used in chromatin immunoprecipitation (ChIP) is shown. ‘F’ represents forward primer and ‘R’ represents reverse primer. (J) Insertional site analysis on single copy MEL clones from uninsulated sBG and insulated sBG-I vector with gene hits according to genome.ucsc.edu.

FIGS. 15A through 15E depict viral titers of lentiviral vectors with inserts into the 3′LTR were inversely proportional to the length of the LTR insert. (A) Schematic representation of the lentiviral vectors. All vectors were based on sBG, a SIN lentiviral vector carrying the β-globin gene, β-globin promoter and the locus control region elements HS2, HS3 and HS4. Different fragments of the cHS4 site were inserted in the U3 region of the sBG 3′LTR (shown above the sBG vector). Similar sized inserts were made by replacing the region downstream of cHS4 core with inert DNA spacers from the lambda phage DNA (shown below the sBG vector). (B) Viral titers of insulated vectors decreased as the length of the insulator insert increased. Titers reflect concentrated virus made concurrently for all vectors in each experiment (n=4). All titers were significantly lower than the titers of the control vector sBG (p<0.01; 1-way ANOVA). (C) Titers fell with insertion of increasing length of an inert DNA spacer downstream of the core. Titers of insulated lentivirus vectors (hatched bars) are similar to those containing inert DNA spacers in the LTR (open bar) in four independent experiments. The titers of sBG with a 400 bp spacer were slightly higher (* p<0.05). (D) The sBG^{2C} vector, carrying tandem repeats of the cHS4 core recombined with high frequency. A schematic representation of the vectors sBG-I and sBG^{2C} proviruses, when intact, or when the core elements recombine with loss of one or two cores with the region probed and restriction site of the enzyme used (AflII) is shown. The size of the expected band is shown adjacent to each vector cartoon. The right panel is the Southern blot analysis showing a single 8 Kb expected band for sBG-I transduced MEL cell population, and two bands in the sBG2.c transduced MEL cell population, representing sBG^{2C} with either loss of one or both cores.

FIG. 16 depicts similar amounts of viral RNA were produced from the insulated and uninsulated vectors in packaging cells. Northern blot analysis on the 293T packaging cells after transfection with sBG and sBG-I vectors showed the expected length viral RNA. The membrane was hybridized with a ³²P labeled p-globin probe (top panel) and 18S (bottom panel) as a loading control. An expected 7.3 Kb and 8.5 Kb band corresponds to sBG and sBG-I viral RNA were detected. The 18S and 28S rRNA was non-specifically probed with this probe. No extraneous recombined bands were detected with either vector. The phosphoimager quantified ratios of viral RNA and 18S rRNA of both vectors are listed below the lanes and show no difference in the amount of v-RNA between the two vectors.

FIGS. 17A through 17D depict virus production was not impaired by insertion of cHS4 in the 3′LTR (A) Reverse transcriptase activity in sBG and sBG-I viral supernatants is similar (23±5 vs. 27±3; n=3, p 0.5). (B) p24 levels detected in the concentrated viral preparation is the same with sBG and sBG-I. (2.9±0.5×105 versus 1.7±0.5×10⁵; n=3, p 0.1) (C) Dot-Blot analysis of viral RNA extracted from sBG and sBG-I viral supernatant shows similar amounts of viral RNA packaged into virions in both vectors. Note that 4 different dilutions of viral RNA were loaded in duplicate for the two vectors. The membrane was hybridized with a 32P labeled p-globin probe. Only one of two representative experiments is shown. (D) Phosphoimager quantification of two independent experiments was plotted and showed similar amounts of viral RNA in sBG and sBG-I virions (1.9±0.7×10⁶ vs. 1.9±0.6×10⁶n=2, p>0.5).

FIGS. 18A through 18D depict kinetic of reverse transcription and nuclear translocation in lentivirus vector carrying insulator element in the LTR. In panel (A) a schema of the lentivirus reverse transcription and nuclear translocation process is illustrated. On the right a summary of q-PCR assays performed to analyze several steps of the process. Thin line: RNA; thick line: DNA. Open boxes: polypurine tract (PPT). Open circle: priming binding site (PBS). The 3′ LTR DNA insert is illustrated in the first strand transfer diagram. The positions of the q-PCR assays are shown. DNA from MEL cells after infection with sBG and sBG-I virus was collected at different time points after infection and analyzed by qPCR. Solid line: sBG. Dashed line: sBGI. (B) Kinetic of reverse transcription before the first strand transfer (R/U5) shows no difference between the two viruses. (C-D) After the first strand transfer (U3/R and Psi) there is a decrease in reverse transcription efficiency in presence of the insulator. (n=3).

FIGS. 19A through 19C depict insertion of cHS4 in the LTR affected viral integration. Linear viral cDNA circularizes and is the form that integrates; 1-LTR and 2-LTR circles represent abortive integration products from homologous recombination and non-homologous end joining, respectively. The 1- and 2-LTR circles are therefore used as markers of nuclear translocation. (A) There are reduced 2-LTR circles, analyzed by qPCR on DNA extracted from MEL cells infected at different times after infection with virus suggesting reduced nuclear translocation or non-homolgous end-joining. (B) Southern blot analysis of MEL cells 72 h after infection with same amount of sBG and sBG-I virus. Stul digestion of genomic DNA allows identification of 1-LTR circles, 2-LTR circles, linear DNA and integrated DNA (a smear) for sBG and sBG-I. Expected band sizes are shown for both vectors. While linear, 1- and 2-LTR circles are seen in the sBG lane, no linear DNA or 2-LTR circles are detected in the sBG-I lane. However, 1-LTR circles are almost as prominent as in the sBG lane. The relative ratios of linear, 1- and 2-LTR circles suggest increased recombined abortive integration products with the sBG-I vector, and hence result in inefficient integration. (C) sBG and sBGI transduced MEL cells show intact proviral integrants (7.5 Kb and 8.0 Kb respectively). There was an 8-fold difference in phosphoimager counts of the two bands. Vector copy number per cell was also quantified by qPCR and is depicted below the lanes.

FIGS. 20A through 20E depict hypothesis of mechanism by which insulator sequence decrease viral titer. In wild type HIV, linear cDNA molecules translocate to the nucleus where a small percentage undergoes recombination and end-joining ligation to form 1- and 2-LTR circles, respectively. Only the linear form is the immediate precursor to the integrated provirus. In the case of insulated LV vectors, it is shown an increase in 1LTR circle formation, due to the presence of a larger U3 sequence that could facilitate an increase in homologous recombination. This process depletes the amount of viral DNA available for integration as well as the amount of 2-LTR circle formation, as shown herein. The decreased amount of DNA available for integration could explain the loss in titers for lentivirus vector carrying large inserts in the LTR. (B) A further addition of a 1.2 Kb PGK-MGMT internal cassette to the BG-I vector, termed BGM-I, did not reduce the titers any further (C) An optimized vector design results in reasonable virus titers without loss of insulator activity. A 650 bp sequence of cHS4, optimized for insulator activity through a structure function analysis. A vector containing this 650 bp fragment (sBG650), was found to have-2-fold lower titers than the uninsulated vector sBG (n=3). (D) PCR for Presence of 3′LTR Inserts in Proviruses Derived from Single Copy MEL Clones Shows Stable Transmission of all Inserts Except those Present as Tandem Repeats MEL cells were cloned from pools with ≤5% gene transfer. Single copy clones were detected using β-globin primers and confirmed by a qPCR using primers spanning the w region. PCR with primers spanning the 250 bp core was performed in the single copy clones, as these core sequences were common to all vectors. The 1.2 Kb cHS4 insert in the sBG-I vector was further confirmed by PCR primers spanning the 5′ core and the 3′ end of cHS4. (E) PCR primers (SEQ ID NOs: 31-43).

FIGS. 21A through 21C depict transgene expression and release during erythroid induction in MEL cells. (A) Illustration of lentiviral vectors. PIHK, erythroid specific hybrid promoter/enhancer containing intron 8 erythroid specific enhancer of human ALAS2 (I), HS40 core element from human alpha LCR (H) and human ankyrin-1 promoter (K); P_{EF}, human EF 1a promoter; PsF, LTR of SFFV; IRES, internal ribosome entry site. (B) Representative FACS plots (left panel), and quantitative analysis (right panel), of GFP expression in transduced MEL before and after inductive culture. Solid bar represents the mean of MFI derived from 2-3 experiments. (C) Intracellular IDUA activities (left panel) and extracellular IDUA release (right panel). Culture media were harvested 24 hr after inoculation of cells at 10⁵ cells/100 ul. All enzyme levels were normalized by transduction efficiency (TE) determined by FACS analysis for GFP±% (mean of 48% for KIiG, 75% for EliG, and 67% for SIiG). Data were derived from 2-3 experiments in duplicate wells and shown as mean±SEM.

FIGS. 22A and 22B depict in vitro erythroid differentiation of MEL cells during HMBA-induction. (A) Morphology changes in MEL cells during inductive culture with 1 mg/ml HMBA. Cytospin slides were counterstained with Wright's dye, demonstrating a gradual reduction of cell volume. (B) Detection of hemoglobin-expressing cells by histochemical staining with Benzidine-hydrogen peroxide solution.

FIGS. 23A and 23B depict erythroid derived IDUA is functional in correcting patient's cells. Lymphoblastoid cells derived from a MPS I patient (LCLmps) were exposed for 3 hr to medium preconditioned by 24 hr culture of enzyme-overexpressing MEL-KEG at Day 7 of induction (30 U/ml IDUA), with or without the presence of 1 mM M6P inhibitor. (A) IDUA specific activity in cell lysate. Data were derived from 2 experiments in triplicate. (B) Normalization of lysosomal abundancy in treated LCLmps. Cells were labeled with lysosome-specific probe (red), and countered stained with DAPI (blue) for nuclei.

FIGS. 24A through 24D depict long-term expression of IDUA in LV-KEG-transduced MPS I chimeras and gene transfer efficiency. (A) Plasma IDUA levels over 5 months after BMT in primary MPS I recipients. MPS I mice were transplanted at 8-9 weeks of age with wild-type BM (MPS/WT), or LV-KIiG-transduced MPS Lin-cells (MPS/KIiG), or LV-EEG-transduced MPS Lin-cells (MPS/EEG). UD, undetectable level of IDUA was found in MPS I mice. Data were derived from 5-7 mice per group. (B) Plasma IDUA levels in secondary MPS I chimeras harboring LV-KIiG or wild-type marrow. Each symbol represents a 2° MPS I BMT recipient, and solid line presents the mean. (C) Transgene frequencies determined by real-time qPCR in PBL and BM from primary and secondary BMT recipients 4-5 months after transplantation. CFU-Spleen assay was conducted with BM from 5 primary donors each into 6-7 secondary mice. (D) IDUA levels in GFP± CFU-S colonies in correlation with vector copy number. Mean of IDUA levels from CFU-S colonies derived from heterozygous (Het) or wild-type (WT) HSCs are also shown.

FIG. 25 (a table) depicts erythrocyte parameters in primary or secondary MPS I chimeras 5-6 months after transplantation, as well as in age-matched MPS I or normal control mice. Complete blood count was performed in primary or secondary MPS I chimeras 5-6 months after transplantation, as well as in age-matched MPS I or normal controls. Data are presented as mean±SD. ^{a} p=0.074, ^{b} p=0.065 and ^{c} p-0.055, in comparison to normal controls.

FIGS. 26A through 26D depict transgene expression pattern in erythroid precursors of primary MPS I BMT recipients. (A) Top panel: representative flow cytogram of BM cells immunostained for Ter119 and CD71, showing gating for various stages of erythroid cells (subpopulation I-IV). Bottom panel: representative histograms for GFP expression in gated sub-populations of treated MPS I mice. Neg, Ter119-CD71-fraction. Background GFP levels in MPS/WT controls are 0.9-1.3% in all subpopulations. (B) Frequency of detectable GFP⁺ cells in various erythroid progenitors. *P<0.05; **P<0.01. (C) Relative expression is shown as fold increase of mean MFI in GFP⁺ cells over GFP-cells in the same subpopulation. N=5 for MPS/KIiG, and n=3 for MPS/WT. (D) Results of GFP analysis.

FIGS. 27A through 27C depict cellular composition of CFU-S colonies and GFP expression in clonal progeny of GFP+ CFU-S. (A) Discrete CFU-S colonies were collected from 6 mice (one KIiG donor into 5 recipients, and one normal donor into 1 control recipient) 12-days after transplantation, followed by immunostaining with erythroid markers CD71 (as C) and Ter119 (as T). Representative cytogram is shown to define non-erythroid cells (C⁻T⁻), early stage erythroblasts (C⁺T⁻), mid/late stage of erythroblasts (C⁺T⁺) and reticulocytes/mature RBC (C⁻T⁺). (B) Cellular compositions of all individual colonies collected (except one lost colony with significantly fewer amounts of cells) are shown. (C) GFP expression in four subpopulations of a GFP⁺ CFU-S colony (dark lines) and a colony from control animal (light dotted lines). Arrows indicate MFI of GFP.

FIGS. 28A through 28M depict long-term systemic correction in treated MPS I mice. (A) Urinary GAG levels were determined by direct DMB dye-binding assay 3-months or 5-months after transplantation. N-4-6 for all groups. ** p<0.002 between MPS I mice and all other groups. (B-M) Representative views of histopathology of liver, spleen and heart from epon-embedded tissue sections (0.5-1 um-thick) with Toluidine blue staining. H, hepatocytes; K, Kupffer cell; P, perisinusoidal cells; I, interstitial cells. For MPS/KIiG and MPS/WT groups, sections of peripheral organs from the animal that exhibited the lowest plasma IDUA among the group are shown.

FIGS. 29A and 29B depict supra-physiological plasma IDUA levels leads to partial CNS correction in MPS I mice. (A) Repeated open-field test. Age-matched untreated MPS I (n=8), KIiG-treated MPS I (n=5), MPS I transplanted with wild-type morrow (n=6) and normal littermates (n=8) were evaluated 5-months after transplantation. Data is presented as mean±SEM. (B) Histopathology of cerebral cortex in epon-embedded tissue sections (0.5 um) with Toluidine blue staining. Representative brain micro-vessels (Mv) (arrowhead) and perivascular cells (Pr) (arrow) are shown in top panel. Bottom panel shows the percentages of Mv that is associated with vacuolated Pr in the brain (% Vac⁺). The mean of scoring data from 6 slides (of two animals) is shown for each group. P<0.01 by Student t-test among all groups.

FIGS. 30A through 30E depict G^{b}Ci mice that underwent transplantation after myeloablative conditioning have high HbF production that is stable and sustained in primary and secondary mice. G^{b}G mice that were fully chimeric for donor RBCs were analyzed at different time points. The proportion of HbF (A) and F cells (B) in blood of individual mice, as determined by ion-exchange HPLC and FACS analysis, respectively, is shown at different time points after primary and secondary transplantations. (C) The amount of HbF in blood directly correlated with the proportion of F cells. (D) The amount of HbF produced was directly in proportion to the vector copy number in bone marrow. Each symbol represents one mouse (and consistently depicts the same particular mouse in all the panels). (E) Hematologic parameters of GbG mice that underwent transplantation after myeloablative conditioning. Hb indicates hemoglobin; MCV, mean corpuscular volume; CH, mean corpuscular hemoglobin; RDW, red cell distribution width; Plt, platelets; pri, primary mice; and see, secondary mice. *P values represent comparison of primary mock mice with the GbG group. Statistical comparisons of secondary mice were not made as only one secondary mock mouse was alive at the time of analysis.

FIGS. 31A through 31H depict G^{b}G mice that underwent transplantation after myeloablative conditioning, which resulted in correction of hematologic parameters that correlated with the HbF expression. There was sustained reduction in reticulocytes (A), and. increase in hematocrit (B) and RBC numbers (C) over time. (D) Leukocytosis decreased with normalization of WBC counts. Data shown represent mean (±SEM) values of G^{b}G mice=5; •) and mice that underwent mock transplantation (n=10; O). A star represents mean values in BERK mice that were HSC donors for the G^{b}G and mock transplantations. (E-G) Decrease in reticulocytes, and increased hematocrit and RBC numbers correlated with the proportion of F cells in individual mice. (H) WBC counts decreased but normalized when the F cells exceeded 60%. WBC counts, counted on an automated analyzer, were representative of circulating leukocytes, since only occasional nucleated RBCs were seen in peripheral smears. Each data point/symbol in panels E-H represents one GbG mouse and symbols for individual mice have been kept consistent, to trace individual mice. A star represents mean values in BERK mice that were HSC donors for the GbG and mock transplantations.

FIGS. 32A through 32G depict GbG mice that underwent transplantation after myeloablative conditioning, which resulted in correction of functional RBC parameters in primary and secondary mice. (A) Peripheral blood smears showing numerous irreversibly sickled cells (ISCs) in a mouse that underwent mock transplantation and a paucity of ISCs in a GbG mouse. (B) Quantification of ISCs in peripheral blood smears of BERK mice that did not undergo transplantation (n=5), mock mice (n=3), and GbG mice (n=5). (*P<0.05; **P<0.01). (C) Deoxygenation of blood induces sick-ling of RBCs in a mock mouse; sickling is largely absent in a GbG mouse. (D) Quantification of sickle RBCs upon graded hypoxia (by tonometry) in the GbG mice (•), compared with mock mice (O). (E) RBC deformability by LORCA analysis in GbG, mock, and normal mice (C57, circle with x in center) analyzed at 18 weeks in primary transplant recipients. Similar data were seen in secondary recipients. Flow at low (3 Pa) and high (28 Pa) shear stress is represented by shaded areas. (F) RBC half-life (determined by in vivo biotin labeling) in the GbG mice, mock/BERK mice, and normal mice after primary transplantations. Similar results were seen in secondary recipients. (G) Correction of organ pathology in GbG mice that underwent transplantation with myeloablative conditioning. 2+ lliver infarction indicates 2 to 3 infarctions/section; 3+ lliver infarction, more than 3 infarctions/section; and E-M, extramedullary. Mild congestion of the spleen vessels with sickle RBCs is seen when splenic architecture is restored. This is not noted when the splenic architecture is effaced by extramedullary erythropoiesis. Splenic erythroid hyperplasia: severe is complete obliteration of splenic follicles; moderate, more than 1 follicle present/section; and mild, preservation of follicles with evidence of erythroid islands. Bone marrow: normal erythropoiesis indicates M/E=5:2; mild erythroid hyperplasia, M/E-2:1; moderate erythroid hyperplasia, M/E=1:1; and severe erythroid hyperplasia, M/E=1:3. Bone marrow erythropoiesis expressed as myeloid-erythroid ratio (M/E). Numbers in parentheses indicate the histologic feature seen in the number of mice/total number of mice analyzed in that group.

FIGS. 33A through 33H depict HbF expression and functional correction in (PG mice that underwent transplantation after reduced-intensity conditioning, separated into 2 groups: mice with HbF of 10% or more (G^{b}G>10) and mice with HbF of less than 10% (GbG<10). (A) HbF in individual BERK mice 18 weeks after transplantation of s G^{b}G-transduced BERK HSCs, after reduced-intensity conditioning. (B-C) Stable and high HbF expression and F-cell repopulation in long-term survivors analyzed at 11 months. (D) Box and whisker plot showing vector copy numbers in G^{b}G<10 and G^{b}G>10 mice, with mean vector copy number denoted by the line. Symbols in panels A through C represent mouse groups: O=mock (HbF 0%), ▾=G^{b}G<10 (HbF<10%), and ●=G^{b}G>10 (HbF>10%). (E) The proportion of ISCs was reduced (P<0.04) in G^{b}G<10 mice, but was markedly reduced in G^{b}G>10 mice (P<0.001), compared with mock mice. (F) Graded deoxygenation via tonometry demonstrates significant reduction in sickling at physiologically relevant partial oxygen pressures (PO2) in G^{b}G>10 mice, whereas G^{b}G<10 mice RBC sickled similar to controls. (G-H) RBC deformability showed highly variable improvement in deformability in G^{b}G<10 mice. In contrast, RBC deformability in G^{b}G>10 mice was highly significantly improved at low and high shear stress (P<0.001). Symbols represent mouse groups: O, mock; ▾, G^{b}G<10; ●, G^{b}G>10; and (circle with x in center), wild-type mice (C57BL/6). Gray shaded rectangles are representative of low and high shear stress through microvessels and large vessels, respectively. Error bars indicate SEM.

FIGS. 34A through 34D depict correction of organ pathology in G^{b}G\hat{ }10 mice that underwent transplantation after reduced-intensity conditioning and improved overall survival. (A) Representative hematoxylin-eosin-stained sections of a kidney, liver, and spleen of G^{b}G>10 and G^{b}G<10 mice 48 to 50 weeks after reduced-intensity conditioning transplantation and a 3-month-old BERK control. Image acquisition nformation is available in supplemental data. (B) Kaplan-Meier survival curve showed significantly improved survival of the G^{b}G>10 mice compared with mock/G^{b}G<10 mice at 50 weeks. Survival at 24 weeks is denoted by a dashed vertical line to compare with survival of the GbG mice in the myeloablative transplantation model. (C) Hematologic parameters of GbG mice that underwent transplantation following reduced-intensity conditioning. Hematologic parameters and abbreviations as stated in the figure. P values represent comparisons of mock mice with G^{b}G>10 at 12 (*), 18 (+), and 24 ($). (D) Organ pathology in GbG mice that underwent transplantation after reduced-intensity transplantation. E-M indicates extramedullary; and 1+liver infarction. 1 infarction/section. *Congestion of vessels and presence of sickle RBC in vessels. Notably, congested vessels were visible in spleens only when erythroid hyperplasia effecting splenic architecture was reduced. The terminology used to quantify organ pathology is the same as documented in the figure.

FIGS. 35A through 35C depict effect of HbF, F cells, and percentage HbF/F cell required for functional improvement in RBC survival and deformability. (A) RBC half-life. Left panel shows a representative G^{b}G mouse injected with biotin with biotin-labeled F cells (upper right quadrants) and non-F cells (lower right quadrants) determined by FACS. Right panel shows survival of F cells (hollow square with solid circle in center), compared with the non-F cells (Hollow circle with solid circle in center) in GbG mice (n=4); wild-type mice (A); and Berkeley mice (O). (B) A cohort of GbG mice analyzed for RBC survival in vivo, based upon the percentage of HbF/Fcell. Each symbol represents a mouse group with HbF percentage and number of mice listed in the adjacent table legend. (C) All GbG and mock mice (n=34) that were analyzed for RBC deformability were divided into groups based on proportion of F cells 0%, 1% to 33%, 33% to 66%, and more than 66%, and deformability of total RBC in these mice was plotted at low (3 Pa, 4) and high (28 Pa, upside down hollow triangle) shear stress. Significantly improved deformability over mock controls is denoted by *(1′)<0.05) and **(1)<0.01). Error bars indicate SEM

FIGS. 36A and 36B depict proportion of transduced HSCs in G^{b}G mice. Proportions in the myeloablative (A) and reduced-intensity (B) transplantation models are shown. The proportion of s G^{b}G-transduced HSCs was determined by spleen colonies (30-36 colonies/mouse) by intracellular staining with HbF and HbS. Each bar represents an individual mouse. (A) In the myeloablative transplantation model, symbols beneath each bar (representing one mouse) are consistent with the symbols in mice labeled. (B) In the reduced-intensity group, bone marrow was successfully aspirated from 8 mice at 24 weeks and mice were followed for an additional 24 weeks. The HbF expression in peripheral blood by HPLC and bone marrow copy number of the respective mice at 24 weeks are labeled under each bar.

FIGS. 37A and 37B depict expansion (A) and cell viability (B) of 293F cell suspension culture over time when initiated at 6×10⁵, 8×10⁵, and 1.5×10⁶ c/mL; mean±SD (n=3).

FIGS. 38A and 38B depict titer of MIEG3 (RD114) produced on 293T and 293F cells transfected using different transfection methods (A); and relative titer of a lentivirus and gamma-retrovirus (LTR and SIN configuration) transfected with lipofectamine (B); mean±SD (n=2). ND, not detected.

FIG. 39 depicts 293T cells (2.5×10⁸) were transfected in a 500 mL FibraStage culture system (New Brunswick Scientific; disposable 500 ml bottle with FibraCel mounted on a movable stage) with 500 microgram of SRS11.SF.GFP.pre*SE, 450 microgram of pCDNA3.MLV.g/p and 200 microgram of GALV envelope plasmid using Calcium Phosphate. One group was transfected at the time of seeding (4 hours post-seeding), the other group was transfected the day after seeding.

FIG. 40 depicts 293T cells were transfected on tissue culture plastic (2×10⁷ cells per T75 in 10 mL D10) or on FibraCel (2×10⁸ cells per 2 gram in 100 mL D10) with SRS11.SF.DsRed2.pre*, pCDNA3.MLV.gp, and Eco-env using different amounts of plasmid DNA (total amount expressed as μg per mL of media). Vector was harvested at 12-hour intervals and titered on NIH 3T3.

FIGS. 41A and 41B depict 293T cells were plated at a cell density of 2.5×104, 5×10⁴, and 1×10⁵ cells/cm² 4 days prior to transfection. At the day of transfection, cells were harvested and 2×10⁸ cells from each group were transfected with a GALV pseudotyped SIN11.SF.eGFP.pre* (A) and SRS11.EFS.IL2RGpre* (B). Vector was harvested at 12-hour intervals and titered on HT1080.

FIGS. 42A and 42B depict 293T cells were transfected T75 (2×107 cells per flask in 10 mL D10) with SERS11.EGFP.pre*, pCDNA3.MLV.gp, and GALV-env. Post-transfection, media was changed at various time points (A). Comparison of PBS rinse followed by 5 min exposure of TrypLESelect and exposure to PBS for 20 min and exposure to TrypLESelect for 30 min, all groups showed >95% viability (B). Average±SD (n=2).

### DESCRIPTION OF THE INVENTION

All references cited herein are incorporated by reference in their entirety as though fully set forth. Also incorporated herein by reference in their entirety are: 1) A description of a determination of the functional portions of the Chicken hypersensitivity site 4 and applications of that determination as described in, “The 3′ Region of the Chicken Hypersensitivity Site-4 Insulator Has Properties Similar to its Core and Is Required for Full Insulator Activity”. (2009) PLOS ONE 4 (9): e6995, Arumugam P, Urbinati F, Velu C, Higashimoto T, Grimes H L, et al. 2) A description of the relationship between reduction in titer and the size of insert into the 3′ LTR in Lentivirus as described in, “Mechanism of Reduction in Titers From Lentivirus Vectors Carrying Large Inserts in the 3′LTR”. Molecular Therapy (2009) 179, 1527-1536. Urbinati F, Arumugam P, Higashimoto T, Perumbeti A, Mitts K, et al. 3) A novel human gamma-globin gene vector for genetic correction of sickle cell anemia in a humanized mouse model and critical determinants for successful correction thereof as described in, “A novel human gamma-globin gene vector for genetic correction of sickle cell anemia in a humanized mouse model: critical determinants for successful correction”. Blood (2009) 114:1174-1185 Perumbeti A, Higashimoto T, Urbinati F, Franco R, Meiselman H et al. 4) The use of erythroid cells as a depot for expressing and excreting corrective enzymes into the blood as described in, “Reprogramming erythroid cells for lysosomal enzyme production leads to visceral and CNS cross-correction in mice with Hurler syndrome.” (2009) PNAS vol. 106 no. 47 19958-19963 (online ahead of print) Wang D, Zhang W, Kalfa T, Grabowski G, Davies S, et al.

Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russel, Molecular Cloning: A Laboratory Mamial 3rd ed., Cold Spring Harbor Laboratory Press (Cold Spring Harbor, NY 2001), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, the term “SIN” is an abbreviation of self-inactivating.

As used herein, the term “HIV” is an abbreviation of human immunodeficiency virus.

As used herein, the term “GFP” is an abbreviation of green fluorescent protein.

As used herein, the term “cDNA” is an abbreviation of complimentary DNA.

As used herein, the term “LTR” is an abbreviation of long terminal repeat.

As used herein, the term “USE sequence” refers to an upstream sequence element.

As used herein, the term “polyA” is an abbreviation of polyadenylation.

As used herein, the term “cHS4” is an abbreviation of chicken hypersensitive site-4 element.

As used herein, the term “MPS” is an abbreviation of Mucopolysaccharidosis.

As used herein, the term “HSC” is an abbreviation of hematopoietic stem cells.

As used herein, the term “GOI” is an abbreviation of gene of interest.

As used herein, the term “HbF” is an abbreviation of fetal hemoglobin.

As used herein, the term “RBC” is an abbreviation of red blood cell. As used herein, the term “IDUA” is an abbreviation of alpha-L-iduronidase.

As used herein, the term “c-GMP” as it relates to virus production is an abbreviation of current good manufacturing practice.

As used herein, the term “MPR” is an abbreviation of mannose 6-phosphate receptor.

As used herein, the term “NTP” is an abbreviation of national toxicology program.

As used herein, the term “MEL Cells” is an abbreviation of murine erythroleukemia cells.

As used herein, the term “LCR” is an abbreviation of locus control region. Essential Cis elements and the optimization of vector design

As described herein, experimentation was conducted to determine whether lentivirus non-coding cis-sequences played a specific role in the RNA export, packaging or expression of β-globin. The vector life-cycle was studied in self-inactivating (SIN)-lentiviruses, carrying the β-globin gene and locus control region (BG), or GFP cDNA. Systematic analysis started with a completely ‘gutted’ minimal SIN-lentivirus carrying only the packaging region; and SIN-lentiviruses containing increasing HIV cis-elements, along with a SIN-gamma-retrovirus. To clone the sSIN-GFP vector, the 3′LTR of a standard SIN-LV backbone previously used, as described herein, was modified to improve transcript termination. Specifically, (β-growth hormone polyadenylation signal was added downstream of the 3′LTR and a USE sequence derived from SV40 late polyadenylation signal was added in the U3 deletion.

As further described herein, SIN-gamma-retrovirus or a gutted/minimal SIN-lentivirus encoding GFP generated high titers and mediated high GFP expression. However, SIN-gamma-retrovirus or the gutted SIN-lentivirus encoding either BG or a similar sized large transgene had barely detectable titers compared to the SIN-lentivirus carrying cis-elements. Systematic addition of cis-elements demonstrated that Rev/RRE was most essential, followed by gag and env splice acceptor sequences, for efficient assembly/packaging of lentivirus particles, not mRNA export. However, these HIV cis-sequences were dispensable for smaller transgenes. These studies identify key lentivirus cis-elements and the role they play in vectors carrying large inserts, and have important implications for gene therapy.

In one embodiment, the present invention provides a method of increasing titer of a modified SIN lentiviral expression vector compared to a standard SIN lentiviral expression vector. In another embodiment, the SIN lentiviral expression vector is modified by inserting a heterologous polyadenylation (polyA) signal sequence downstream from a viral 3′ long terminal repeat sequence in a standard SIN lentiviral vector backbone. In another embodiment, the polyA signal is the bovine growth hormone polyA signal sequence. In another embodiment, the SIN lentiviral vector is modified by inserting one or more of an upstream polyA-enhancer sequence (USE sequence) into a 3′LTR of a standard SIN lentiviral vector backbone. In another embodiment, the USE sequence is derived from the SV40 late polyA signal. In another embodiment, 2-10 copies of the USE sequence are inserted into a 3′LTR of a standard SIN lentiviral vector backbone. In another embodiment, 3-5 copies of the USE sequence are inserted into a 3′LTR of a standard SIN lentiviral vector backbone. In another embodiment, one or more copies of the USE sequence is inserted into the U3 region. In another embodiment, the β-growth hormone polyA signal and one or more copies of the USE sequence derived from the SV40 late polyA signal are both incorporated into the expression vector. In another embodiment, the expression vector contains a gene of interest (GOI). In another embodiment, the gene is operably linked to a promoter. In another embodiment, the promoter is a lineage-specific promoter. In another embodiment, the promoter is an erythroid specific promoter. In another embodiment, of the GOI is β-globin. In another embodiment, the GOI is gamma-globin. In another embodiment of the invention the gamma-globin gene is under the control of β-globin regulatory elements. In another embodiment, the vector is used to treat sickle cell anemia via gene therapy. In another embodiment, the vector is used in conjunction with reduced intensity conditioning to treat sickle cell anemia. In another embodiment, the SIN lentivirus comprises a bovine, equine, feline, ovine/caprine or primate derived variety of lentivirus. In another embodiment, the SIN lentivirus is an HIV derived SIN lentivirus. In another embodiment the modified SIN lentiviral vector is introduced into a eukaryotic cell by transfection.

In one embodiment, the present invention provides a method of designing a gutted/minimal, and thus less recombinigenic and safer SIN lentiviral vector for the expression of small therapeutic transgenes that do not require extensive Cis elements for efficiency. In another embodiment the small therapeutic transgenes are equal in size or smaller than green fluorescent protein (GFP). In another embodiment the small therapeutic transgenes are smaller than human β-globin.

### Chromatin Insulator Elements

As described herein, chromatin insulator elements prevent the spread of heterochromatin and silencing of genes, reduce chromatin position effects and have enhancer blocking activity. These properties are desirable for consistent predictable expression and safe transgene delivery with randomly integrating vectors. Overcoming chromatin position effects can reduce the number of copies required for a therapeutic effect and reduce the risk of genotoxicity of vectors. Vector genotoxicity has become an area of intense study since the occurrence of gene therapy related leukemia in patients in the X-SCID trials. Gamma-retroviral vectors and lentiviral vectors have been modified to a self-inactivating (SIN) design to delete ubiquitously active enhancers in the U3 region of the long terminal repeats (LTR). A 1.2 Kb DNAse hypersensitive site-4 (cHS4) from the chicken p-globin locus has been inserted in the 3′LTR to allow its duplication into the 5′LTR in gamma-retrovirus and lentivirus vectors. Insulated vectors have reduced chromatin position effects and, provide consistent, and therefore improved overall expression. A side-by-side comparison of cHS4 insulated and uninsulated lentivirus vectors carrying hβ-globin and the locus control region was performed, and resulted in the discovery that insulated vectors showed consistent, predictable expression, regardless of integration site in the differentiated progeny of hematopoietic stem cells, resulting in a 2-4 fold higher overall expression. Recent evidence also suggests that cHS4 insulated lentivirus vectors may reduce the risk of insertional activation of cellular oncogenes. Despite the beneficial effects of insulated vectors, they also lead to a significant reduction in titers with insertion of the full-length 1.2 Kb cHS4 insulator element in the 3′LTR of lentivirus vectors. There are similar reports of lowering of viral titers or unstable transmission with gamma-retrovirus vectors containing insertions in the 3′ LTR. This reduction in titers becomes practically limiting for scale up of vector production for clinical trials, especially with vectors carrying relatively large expression cassettes, such as the human β-globin gene (hβ) and locus control region (LCR), that have moderate titers even without insulator elements.

The effects of insertions of exogenous fragments into the LTR on viral life cycle have not been addressed. The mechanism by which insertion of cHS4, or other inserts in the viral 3′LTR lower titers of lentiviral vectors was therefore studied. Large LTR inserts lower titers via a post-entry restriction in reverse transcription, and increased homologous recombination in the LTRs of viral cDNA, thus reducing the amount of virus DNA available for integration. These results have important implications for vector design for clinical gene therapy. Studies on the chicken hypersensitive site-4 (cHS4) element, a prototypic insulator, have identified CTCF and USF-1/2 motifs in the proximal 250 bp of cHS4, termed the “core”, which provide enhancer blocking activity and reduce position effects. However, the core alone does not insulate viral vectors effectively. While the full-length cHS4 has excellent insulating properties, its large size severely compromises vector titers. A structure-function analysis of cHS4 flanking lentivirus-vectors was performed and transgene expression in the clonal progeny of hematopoietic stem cells and epigenetic changes in cHS4 and the transgene promoter were analyzed.

As further described herein, the core only reduced the clonal variegation in expression. Unique insulator activity resided in the distal 400 bp cHS4 sequences, which when combined with the core, restored full insulator activity and open chromatin marks over the transgene promoter and the insulator. These data consolidate the known insulating activity of the canonical 5′ core with a novel 3′ 400 bp element with properties similar to the core. Together, they have excellent insulating properties and viral titers. This data has important implications with respect to understanding the molecular basis of insulator function and design of gene therapy vectors.

In one embodiment, the present invention provides a method of increasing the titer of lentiviral vectors by incorporating one or more reduced-length chromatin insulators containing functional portions of a full-length chromatin insulator. In another embodiment, the functional portions are derived from a single type of full length chromatin insulator. In another embodiment, the reduced-length functional insulator comprises functional portions of two or more separate varieties of chromatin insulators. In another embodiment, the functional reduced-length chromatin insulator is derived from a chicken hypersensitive site-4 (cHS4) element. In another embodiment, the functional reduced-length insulator is a cHS4-derived insulator of 650 base pairs or less. In another embodiment, one or more reduced-length cHS4-derived insulators is combined with other modifications to a SIN lentivirus expression vector in order to increase titer and improve stability of transgene expression. In another embodiment, one or more reduced-length cHS4-derived insulators is added to a vector containing a heterologous polyadenylation (polyA) signal sequence downstream from a viral 3′LTR and a USE sequence in the U3 deletion.

### Erythroid Cells Function as an Effective Depot

As disclosed herein, restricting transgene expression to maturing erythroid cells can reduce the risk of activating oncogenes in hematopoietic stem cells (HSCs) and their progeny, while taking advantage of their robust protein-synthesis machinery for high-level protein production. An erythroid-specific hybrid promoter can provide inducible expression and release of a lysosomal enzyme, alpha-L-iduronidase (IDUA), during in vitro erythroid differentiation in murine erythroleukemia cells. The erythroid released IDUA can use the MPR lysosomal enzyme trafficking system and can lead to phenotypic cross-correction in an enzyme-deficient lymphoblastoid cell line derived from patients with Mucopolysaccharidosis (MPS) Type I. Stable and higher than normal plasma IDUA levels were achieved in vivo in primary and secondary MPS I chimeras for at least 9 months after transplantation of HSCs transduced with the erythroid-specific IDUA-containing lentiviral vector (LV). Moreover, long-term metabolic correction was demonstrated by normalized urinary glycosaminoglycan accumulation in all treated MPS I mice. Complete normalization of tissue pathology was observed in heart, liver and spleen. Notably, neurological function and brain pathology were significantly improved in MPS I mice by erythroid-derived, higher-than-normal peripheral IDUA protein.

As further disclosed herein, these data are the first to demonstrate that late-stage erythroid cells, transduced with a tissue-specific LV, can deliver a lysosomal enzyme continuously at supra-physiological levels to the bloodstream, and can correct the disease phenotype in both viscera and CNS of MPS I mice. This approach provides a paradigm for the utilization of red blood cell precursors as a depot for efficient and potentially safer systemic delivery of non-secreted proteins by ex vivo HSC gene transfer.

In one embodiment, the present invention provides a method of genetically modifying an erythroid cell for utilization as a depot to produce and expel proteins non-native to blood and/or not conventionally secreted into blood circulation. In another embodiment, modification is accomplished by transduction of an HSC with a vector capable of providing long-term gene transfer. In another embodiment, the vector comprises an erythroid specific promoter and a gene of interest (GOI) operably linked to the promoter. In another embodiment, the GOI encodes a protein non-native to erythroid cells and/or not conventionally secreted. In another embodiment, activation of the erythroid specific promoter leads to expression and expulsion by an erythroid cell offspring of a protein or proteins non-native to erythroid cells and/or not conventionally secreted by erythroid cells. In another embodiment, the expression results in a sustained release of high levels of the protein in the blood circulation.

In one embodiment, the present invention provides a method of genetically correcting a lysosomal storage disease and/or reducing symptoms thereof. In another embodiment, the genetic correction and/or reduction in symptoms is accomplished by transducing a HSC with a vector comprising an erythroid specific promoter and a gene encoding a corrective enzyme in an individual with the lysosomal storage disease, wherein the gene is operably linked to the promoter. In another embodiment, activation of the erythroid specific promoter leads to expression and expulsion of the corrective protein by an erythroid cell offspring, resulting in correction of the disease and/or a reduction in symptoms. In another embodiment, the lysosomal storage disease is MPS Type I. In another embodiment, the lysosomal storage disease is Hurler Syndrome. In another embodiment of the invention the GOI encodes IDUA. In another embodiment, the expelled protein is a variety imported into cells via receptor-mediated endocytosis. In another embodiment, the mannose 6-phosphate receptor (MPR) pathway mediates trafficking of the corrective enzyme. In another embodiment, release of the corrective enzyme into the bloodstream corrects or improves CNS abnormalities associated with a lysosomal storage disease.

### Determining Critical Parameters of Disease Correction-Sickle Cell Anemia

As disclosed herein, lentiviral delivery of human γ-globin under β-globin regulatory control elements in HSCs results in sufficient postnatal HbF expression to correct SCA in mice. The amount of HbF and transduced HSCs was then de-scaled, using reduced-intensity conditioning and varying multiplicity of infection (MOI), to assess critical parameters needed for correction. A systematic quantification of functional and hematologic RBC indices, organ pathology, and life span were critical to determine the minimal amount of HbF, F cells, HbF/F cell, and gene-modified HSCs required for reversing the sickle phenotype.

As further disclosed herein, amelioration of disease occurred when HbF exceeded 10%, F cells constituted two-thirds of the circulating RBCs, and HbF/F cell was one-third of the total hemoglobin in RBCs; and when approximately 20% sG^{b}G modified HSCs repopulated the marrow. Genetic correction was sustained in primary or secondary transplant recipients followed long-term. The present study describes a method of determining minimum HSC chimerism for correction of a hematopoietic disease in an in vivo model, which would contribute to design of cell dose and conditioning regimens to achieve equivalent genetically corrected HSCs in human clinical trials. Moreover, this study addresses, for the first time, the gene dosage and the gene-modified hematopoietic stem cell dosage required for correction of a genetic defect.

In one embodiment, the present invention provides a method of determining minimum HSC chimerism for correction of a hematopoietic disease in an in vivo model. In another embodiment, reduced intensity conditioning prior to transplantation is used as a method of varying HSC chimerism. In another embodiment, the proportion of transduced HSCs and vector copy/cell is varied by transducing the cells at a range of MOI (30-100). In another embodiment, the MOI is 20-120. In another embodiment, the minimum determined chimerism and gene dosage can be used to design cell dose and conditioning regimens to achieve equivalent genetically corrected HSCs in human clinical trials. In another embodiment, reduced intensity conditioning is used prior to transplantation in a clinical setting to reduce transplantation-related morbidity. In another embodiment, the hematopoietic disease is sickle cell anemia. In another embodiment, the hematopoietic disease is 0-thalassemia.

### Improved Vector Production

As disclosed herein, the need for clinical grade gamma-retroviral vectors with self-inactivating (SIN) long terminal repeats has prompted a shift in the method with which large scale cGMP-grade vectors are produced, from the use of stable producer lines to transient transfection-based techniques. A method was developed based on the Wave Bioreactor® (GE Healthcare) production platform. This platform allows for large-scale closed-system production of high-titer retroviral vectors for clinical trials using transient transfection up to 25 Liters per harvest using closed system processing. The present study describes the development and scale-up procedures and reports on the successful use of the Wave Bioreactor in the production of six cGMP grade retroviral vectors in support of the FDA's National Toxicology Program (NTP).

As further disclosed herein, in order to determine the optimal time of transfection, 293T cells were seeded onto FibraCel and exposed to transfection reagents and plasmid DNA within hours of seeding as compared to cells that were transfected the following day. The data show a titer of less than 10⁴ IU/mL from cells that were transfected one day post-seeding as compared to cells that were transfected the same day. It has now been determined that optimal titers are achieved when cells are mixed with transfection reagents and plasmid DNA at the time of seeding onto FibraCel. Cells were plated at different cell densities, harvested and tested for virus production in five separate experiments using GALV pseudotyped gamma-retroviral vectors. Although the same number of cells was used for each group, titers varied greatly based on the plating density and were higher when cells were harvested from plates that had been seeded with a higher cell density. For scale-up, several parameters were tested including the time of media change post-transfection and the length of time the cells were exposed to PBS and TrypLESelect prior to transfection. To establish the amount of plasmid DNA necessary to improve titer, 293T cells were transfected side-by-side on tissue culture plastic as well as FibraCel. Where increasing plasmid DNA in static cultures produced a lower titer, increasing the DNA concentration on FibraCel increased titer.

In one embodiment, the present invention provides a method of improving viral titer in a transfection-based production system using eukaryotic cells. In another embodiment, the cells harvested prior to transfection have progressed beyond log phase of cell growth. In another embodiment the cells have achieved a state of confluency for at least 24 hours. In another embodiment, the cells are seeded at a cell density of at least 5×10⁴ 4 to 5 days prior to cell harvest and transfection. In another embodiment the cells are mixed with transfection reagents and plasmid DNA at the time of re-seeding into a new culture vessel. In another embodiment, the plasmid concentration used for transfection is at least 7 μg/ml of plasmid DNA. In another embodiment, the plasmid concentration used for transfection is at least 9.2 μg/ml of plasmid DNA. In another embodiment, the media is changed 12-24 hours post-transfection. In another embodiment, the media is changed 14-20 hours post-transfection. In another embodiment, the media is changed 19 hours post-transfection. In another embodiment, cells are rinsed with PBS followed by 3-8 minute exposure to TrypLESelect prior to transfection. In another embodiment, cells are rinsed with PBS followed by 4-7 minute exposure to TrypLESelect prior to transfection. In another embodiment, cells are rinsed with PBS followed by 5 minute exposure to TrypLESelect prior to transfection. In another embodiment, the harvesting, mixing, re-seeding, and/or transfection steps, alone or in combination, results in improved viral titer compared to traditional protocols of transfection-based production using eukaryotic cells. In another embodiment, the cells are 293T cells. In another embodiment, the vector is a SIN lentiviral vector. In another embodiment, the vector is a Gamma-Retroviral vector. In another embodiment, the vector is a SIN Gamma-retroviral vector. In another embodiment, the retroviral vectors produced are cGMP grade vectors. In another embodiment, the vectors are produced in a closed system bioreactor.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Lentivirus Cis Elements Required for Efficient Packaging of Large Transgenes Cassettes Like β-Globin

This study investigated whether lentivirus non-coding cis-sequences played a specific role in the RNA export, packaging or expression of β-globin. The vector life-cycle was studied in self-inactivating (SIN)-lentiviruses, carrying the β-globin gene and locus control region (BG), or GFP cDNA. Systematic analysis started with a completely ‘gutted’ minimal SIN-lentivirus carrying only the packaging region; and SIN-lentiviruses containing increasing HIV cis-elements, along with a SIN-gamma-retrovirus. It was discovered that (i) SIN-gamma-retrovirus or a gutted/minimal SIN-lentivirus encoding GFP generated high titers and mediated high GFP expression. (ii) However, SIN-gamma-retrovirus or the gutted SIN-lentivirus encoding either BG or a similar sized large transgene had barely detectable titers compared to the SIN-lentivirus carrying cis-elements. (iii) Systematic addition of cis-elements demonstrated that Rev/RRE was most essential, followed by gag and env splice acceptor sequences, for efficient assembly/packaging of lentivirus particles, not mRNA export. However, these HIV cis-sequences were dispensable for smaller transgenes. These studies identify key lentivirus cis-elements and the role they play in vectors carrying large inserts, and have important implications for gene therapy.

### Example 2

BG Expression from Gutted SIN-γRV

It has been postulated that γRV are unable to successfully express hβ-globin due to transcriptional interference between the strong γRV LTR promoter/enhancer elements and the internal LCR enhancer. SRS11.SF is a SIN-γRV that encodes the GFP cDNA under control of an internal Spleen Focus-Forming Virus (SFFV) promoter/enhancer. The SFFV-GFP in SRS11.SF was replaced with BG, an expression cassette that was successfully utilized in a standard SIN-LV to achieve therapeutic h β-globin expression in thalassemia, to generate SRS11.BG. The rationale for using SRS. 11, despite the notoriety of β-globin γRV was: (i) it contains the minimal packaging region (4), lacks gag sequences and can carry a larger vector payload, yet retains extremely high titers; (ii) it carries a large 400 bp U3 deletion of the 3′LTR, comparable to the deletion in SIN-LV. (iii) Large LCR elements have never been tested in γRV due to restrictions on vector payload.

Infectious titers and expression of SRS11.BG and SRS11.SF γRV vectors were compared on the murine erythroleukemia (MEL) cell line. Human p-globin protein expression was almost undetectable from SRS11.BG-transduced MEL cells, in contrast to the high expression of GFP in cells transduced with SRS.11 SF. The unconcentrated viral titers of SRS11 BG versus SRS11.SF vector were 6.8±5×10³ IU/mL versus 4±0.2×10⁶ IU/mL. Viral RNA (vRNA) transcripts were barely detectable in 293T cells with the SRS11.BG via northern blot analysis (data not shown). Therefore, production of BG vRNA and viral particles from γRV, even those optimized for a SIN design and high vector payload was severely impaired.

### Example 3

Expression of Large Small Transgenes from Standard or Gutted Minimal LV

In contrast to the SIN-γRV used herein, the “standard” SIN-LV commonly used retains relatively large portions of viral sequences amounting to about 20-25% of the HIV genome. These cis elements are: the LTR (634 bp for wt HIV LTR or 235 bp for SIN-LV LTR), the packaging signal ψ(150 bp), 5′ portion of the gag gene (300 or 600 bp), env sequences including the rev response element (RRE, 840 bp) and the central flap/polypurine tract (cPPT) from the pol gene (120 bp).

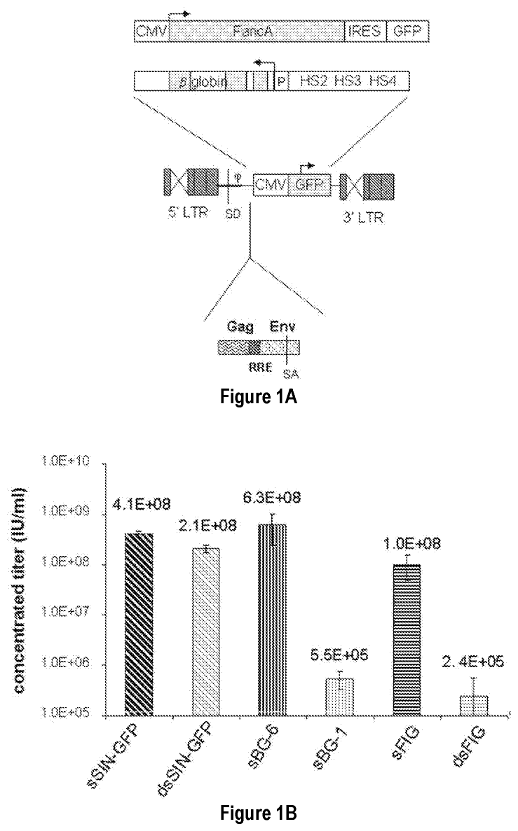

To examine the requirement of cis-sequences for GFP versus BG, the CMV-GFP cassette was cloned in a) the “standard” SIN-LV containing cis sequences listed above (sSIN-GFP), and b) a ‘gutted’ minimal SIN-LV where the gag, RRE and the rest of the env sequences were deleted and only the & region was retained (dsSIN-GFP; FIG. 1A). The titers of the minimal dsSIN-GFP LV were only 2-times lower than the titers of the “standard” LV sSIN-GFP FIG. 1B; p<0.01; n=3. In sharp contrast to the GFP vectors, the difference in titers of the analogous standard and gutted BG SIN-LV, sBG-6 and sBG-1 vectors was 1100-fold p<0.01; n=4; (FIG. 1B). Clearly, the LV non-coding sequences are necessary either for production of LV particles and/or for β-globin expression; and these sequences have a pronounced effect on infectious titers of LV encoding the β-globin gene, but not those encoding GFP. Next, vectors were constructed with a similar size transgene cassette, CMV-FANCA-IRES-GFP (FIG) as sBG (FIG. 1A) in the “standard” (sFIG) or the gutted (dsFIG) SIN LV. The same dependence of FIG on LV cis sequences: titers of dsFIG vector were three orders of magnitude lower than those of sFIG were observed (FIG. 1B). Therefore LV cis elements are dispensable for small inserts, but necessary for high titers of large inserts.

### Example 4

### LV Constructs Designed to Study the Role of Cis-Sequences

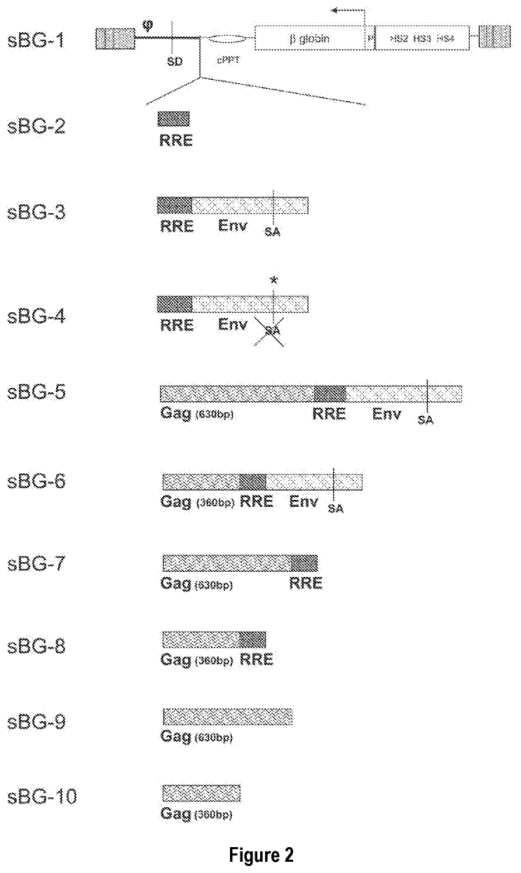

To study which particular LV cis sequences were important for this effect, and what step of the vector life cycle they affected, a series of ten SIN-LV vectors were cloned; all of them carrying the BG cassette but carrying different lentiviral non-coding cis elements (FIG. 2). The rationale for studying specific env (RRE and SA) and gag sequences in the context of BG was: (i) The RRE element in the env fragment in a “standard” LV facilitate transport of unspliced/singly spliced transcripts from the nucleus following binding with the Rev protein. (ii) The env splice sites play a fundamental role in the stability of vRNA and its availability for packaging, and absence of known downstream splice acceptor (SA) sequences results in cis-acting repressor sequence (CRS) activity, which hinders cytoplasmic accumulation of HIV-1 RNA. (iii) A portion of the gag gene is retained in vectors to help vRNA packaging. Gag sequences promote folding of the RNA secondary structure of the packaging signal, facilitate the interaction of vRNA with Gag proteins during particle formation, and are important for the dimerization of the vRNA. Sequences mapped to the 5′ splice donor site and the first 360 bp of the gag gene direct unspliced and singly spliced viral mRNA to specific subnuclear compartments from where it is exported with the help of Rev/RRE.

The first vector (sBG-1) maintained only the packaging signal (containing the 5′ splicing donor site) and the cPPT/flap (FIG. 2). Starting from this vector, the RRE, the rest of the env fragment containing the SA, and two different size gag fragments (360 bp and 630 bp) were sequentially cloned into sBG-2, sBG-3, sBG-5 and sBG-6. To verify the activity of the splicing acceptor (SA) the sequence in the env fragment was mutated by PCR site-specific mutagenesis (sBG-4). In the last four vectors, the entire env fragment including the RRE was first removed, leaving only the long and short version of the gag fragments (sBG-9, sBG-10); or additionally added RRE (sBG-7, sBG-8) downstream of the long and short gag fragments.

### Example 5

Viral Titers with Inclusion of Different HIV Cis Sequences

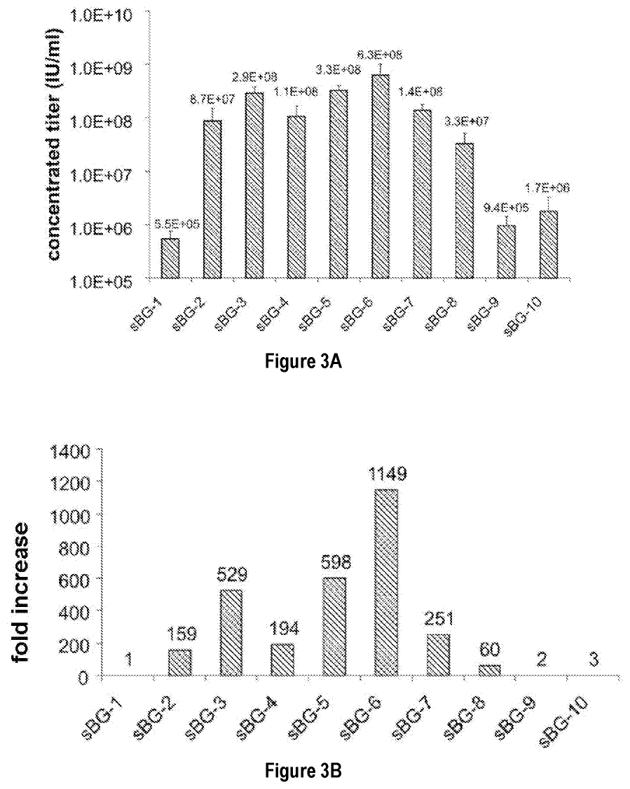

The vectors without the RRE element (sBG-1, sBG-9 and sBG-10) had a concentrated titer ranging from 5.5±2.1×10⁵ IU/mL to 1.7±1.4×10⁶ IU/mL, which was 2-3 orders of magnitude lower than vectors that carry the RRE sequence (sBG-2 to sB G-8; p<0.01). Indeed when only the RRE sequence was added to sBG-1 to generate sBG-2, the titer increased by more than a 100-fold (5.5±2.1×10⁵ IU/mL versus 8.7±6.5×10⁷ IU/mL; p<0.01; FIG. 3).

Addition of the env fragment containing the SA site increased vector titers 3-5 fold: 2.9±0.9×10⁸ IU/mL for sBG-3 versus 8.7=0.7×10⁷ IU/mL for sBG-2 (p<0.01). This effect was specific to the SA, since titers of sBG-4 vector, which contains the env sequence with a mutated SA were 1.1±0.61×10⁸ IU/mL, and were similar to that of sBG-2 carrying only the RRE (sBG4 vs. sBG-3 p<0.01). The addition of a long and short fragment of gag to env (RRE and SA) containing vectors sBG-5 and sBG-6, respectively, showed a further increase in titers by −4-5 fold, with titers from sBG-6 reaching 6.3×10⁸ IU/mL (sBG-4 vs. sBG-5 and sBG-6 p<0.01). The data suggested that the longer portion of gag was not necessary for high BG titers. However, titers of vectors carrying only the short/long gag fragments, without the RRE and env SA were low (sBG-9 and sBG-10), as compared to those containing the RRE as well (sBG-7 and sBG-8; p<0.01). Titers of sBG-7, 8, 9, and 10 ranged from 9.4±4.7×10⁵ IU/mL to 1.4±0.4×10⁸ IU/mL. Titers improved further by 3-5 fold with the inclusion of env SA. Thus, the gag fragment alone, or the combination gag/RRE was not sufficient to confer optimal titers to BG vectors, suggesting HIV-1 cis sequences acted cooperatively.

To study whether the strong effect of the RRE on viral titers was Rev-dependent, the sBG-6 vector was packaged with and without Rev. In these experiments, the packaging system was changed from 3-plasmid to a 4-plasmid system, wherein Rev and Gag-Pol were provided from different plasmids. The titers of sBG-6 were approximately 400-fold higher with Rev (3.8±0.3×10⁷ IU/mL) than without the Rev protein (9.4×±5.8×10⁴ IU/ml; p<0.01), showing that interaction of Rev with RRE was necessary for high titers.

Taken together, these data indicate that HIV-1 Rev/RRE, gag and env SA were critical for high titers of LV carrying a large cargo such as BG or FIG, although they are dispensable for small GFP based cassettes.

### Example 6

### Role of LV Cis-Elements in the Vector Life Cycle

In order to assess the role of LV cis-elements in proviral stability and expression a genomic Southern blot analysis on transduced MEL cells was performed.

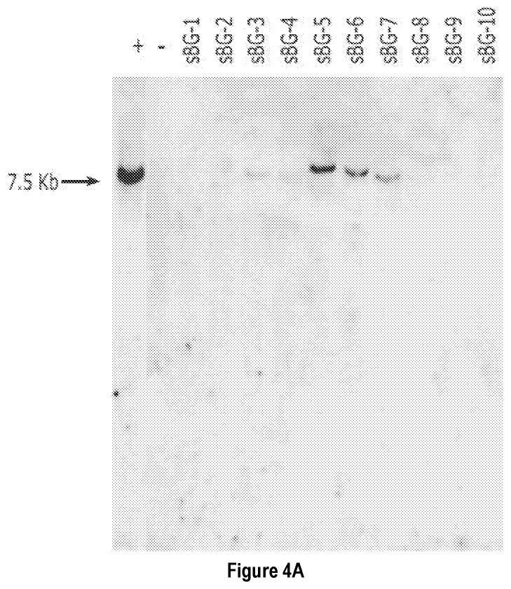

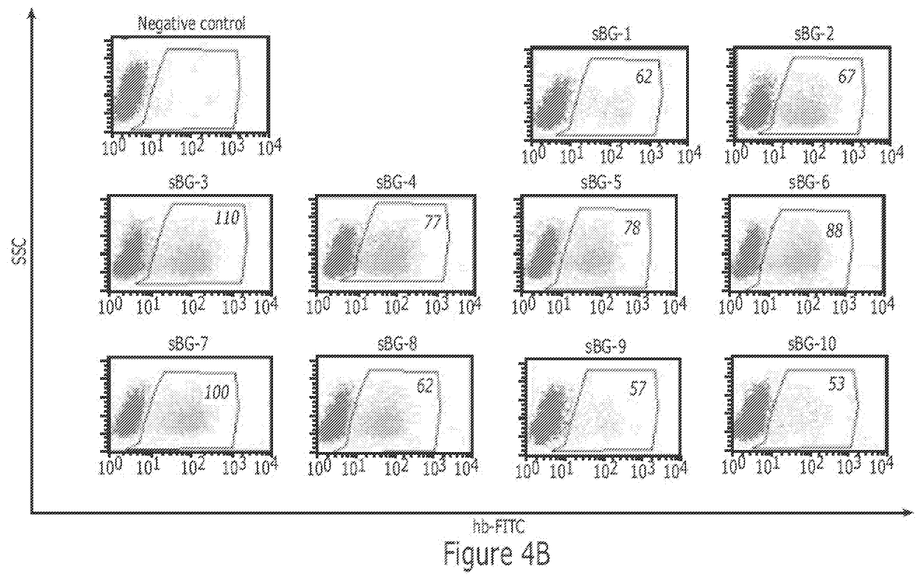

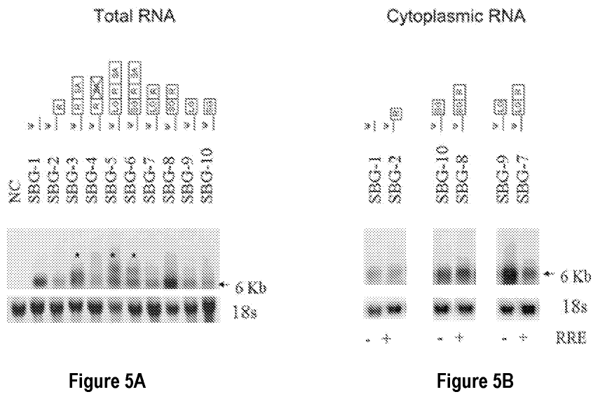

Surprisingly, given previous difficulties with genomic rearrangements of hf3-globin-containing γRV, only one proviral band of the expected size was detected in most of the LV FIG. 4A. Some low titer vectors were undetectable at the level of sensitivity of a Southern blot. Subsequent northern blot analysis in packaging cells confirmed that the expected full-length vRNA transcripts were generated from all LV (FIG. 5A), confirming that LV carrying the large BG cassette do not require cis-sequences for stable transmission.

In order to determine whether LV cis-sequences affected the level of expression of integrated BG proviruses, MEL cells were transduced with vectors sBG-1 through sBG-10 at a range of multiplicity of infection. Mean fluorescence intensity (MFI) was compared in MEL cell pools with a similar percentage of hβ-globin expressing cells (15-20%), except in vectors with low titers, where only a small percentage of gene transfer could be achieved. The MFI of the transduced MEL cell population was comparable among all the vectors (ranging from 62 to 110 arbitrary units), including that of the low titer vectors (FIG. 4B). Thus, LV cis-elements did not play a major role in regulating the expression of BG.

In order to determine the role of RRE, gag and env SA in vRNA production and cytoplasmic export the steps of vector life cycle that could impair generation of full-length vRNA in the packaging cells, its subsequent cytoplasmic export, assembly and packaging into vector particles was studied.

Total, cytoplasmic and nuclear RNA was fractionated from 293T packaging cells transfected with sBG-1 through sBG-10. FIG. 5A shows a northern blot analysis on total RNA probed with hβ-globin probe. Correctly size bands of intact vRNA from all the vectors, including the vectors without the RRE were determined (sBG-1, sBG-9 and SBG-10). The spliced and unspliced vRNA transcripts were only present for the vectors sBG-3, sBG-5 and sBG-6, since these vectors carry the env SA site. Thus, no appreciable aberrant splicing occurred in any of the LV backbones, confirming lack of recombination of the hβ-globin gene and LCR elements, and contrasting results reported with γRV.

Significantly, all vectors with very low titers, including sBG-1, sBG-9 and sBG-10 that do not contain RRE, produced vRNA in quantities that were comparable to, or higher than the highest titer vectors (sBG-5 and sBG-6). Since this finding was unexpected, the northern blot was repeated in a separate experiment, with fractionation of total and cytoplasmic RNA, with identical results.

Rev/RRE has been best characterized for export of full-length vRNA to the cytoplasm. Therefore, the next step was to determine if RRE contributed to high titers via vRNA export. Northern blot analysis showed similar amounts of vRNA in the cytoplasm of analogous vectors without or with RRE (sBG-1 versus sBG-2, sBG10 versus sBG-8, and sBG-9 versus sBG-7; FIG. 5B). The ratio of cytoplasmic RNA to total RNA in northern blots from two separate experiments is shown in 6E. The cytoplasmic vRNA transcripts were only 2-fold higher in sBG-2, when compared to sBG-1. The converse was seen with sBG-10 and sBG-9 vectors, where cytoplasmic vRNA transcripts were ˜2-fold higher than analogous vectors sBG-8 and sBG-7, which contained the RRE. Since the difference in titers between vectors with and without the RRE was 2-3 orders of magnitude, RRE likely played a minimal role in increasing nuclear export of vRNA transcripts via these vectors.

### Example 7

### LV Cis-Elements, Including RRE Improve Packaging

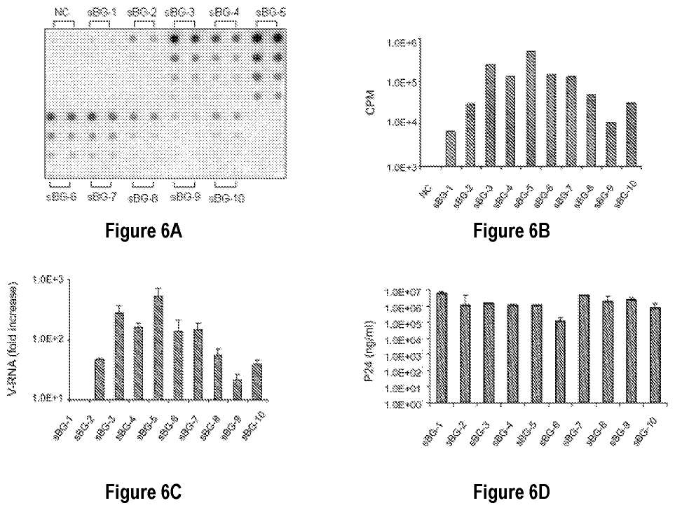

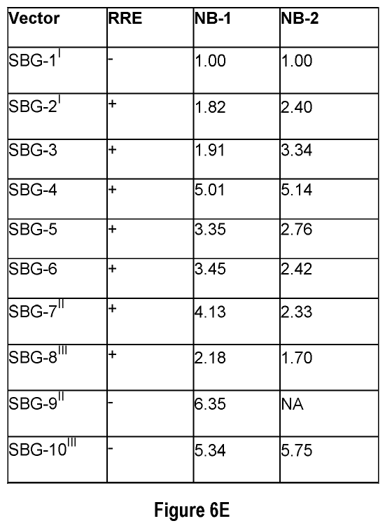

The effect of cis sequences on the packaging efficiency was next determined by analyzing vRNA, p24 levels and viral associated reverse transcriptase (RT) in purified virus particles from all ten vectors processed identically. FIG. 6A shows a representative dot blot analysis of sBG1 through sBG-10 LV. The amount of vRNA detected is proportional to the vector titer for most of the vectors, as determined by phospho-imager analysis, indicating a block in packaging efficiency in vectors lacking cis-sequences (FIG. 6B-C).

There were some exceptions that suggested cis-sequences may have some effect on steps following target cell entry: RNA in 293T cells and the infectious titers of sBG-2 and sBG-4 were comparable, although sBG-4 vRNA was 4-times higher. It seems that sBG-4 vRNA, even when packaged more efficiently, may not be stable post-target cell entry due to the absence of env SA, which is known to stabilize RNA. sBG-6 and sBG-7 had the same amount of vRNA but the titer of sBG-7 was 4-5 times lower; here again sBG-7 did not have the env SA. sBG-5, containing the inhibitory region of gag, had higher vRNA, but lower titers.

Overall, the amount of BG vRNA packaged in viral particles correlated with the transduction/infectious titers in target cells, despite high levels of mRNA produced in packaging cells with all 10 vectors. The p24 activity was similar in all the concentrated virus preparations (FIG. 6D), suggesting that viral like particles (containing no vRNA) were formed efficiently with all vectors.

### Example 8

### γRV and LV Size, Payload and Titer

Titers of the standard LV carrying BG are low to begin with, and require extensive concentration. However, the titers fall precipitously (by three orders of magnitude) with the removal of LV cis-elements. Perhaps these LV sequences protect large vRNA from degradation in packaging cells while promoting assembly, while the short GFP vRNA gets efficiently packaged without such requirements. The low titer of the ‘gutted’ BG LV are not from anti-sense RNA arising from the β-globin gene promoter inserted in the reverse orientation with respect to the 5′LTR vRNA transcript in 293T cells. There was no antisense transcript in the northern blot with any of the vectors. Besides, β-globin transcripts are erythroid-specific, and are not produced in 293T cells. Furthermore, the FIG cassette that was similar in size to BG, but in sense orientation also had the same effect on titers as BG.

### Example 9

### Cis Elements and Vector Life-Cycle

Several unique, rather unexpected results emerged from this study: (i) in packaging cells, large amounts of transcripts were produced with all BG LV in contrast to barely detectable RNA with BG γRV. One possibility is that LV minimal sequences (R, U5 and ψ regions) confer stability to BG vRNA in specific sub-cellular compartments. Therefore, high amounts of vRNA are seen in 293T cells even from the gutted LV, an essential difference from the BG γRV. (ii) BG vRNA was of the expected size and efficiently exported into the cytoplasm even in the absence of Rev/RRE, contradicting the belief that the success of ‘globin genes’ in LV is secondary to the archetypal functions of RRE of preventing splicing and vRNA export. (iv) vRNA was efficiently packaged into virions when the gutted LV encoded a small transgene such as GFP. This data confirms LV cis-sequences, other than the minimal packaging sequence, are dispensable for small transgenes.

### Example 10

### Role of RRE in Packaging

Rev/RRE interaction was most critical for packaging and high titer virus production, while the well-established function of Rev/RRE in the export of the genomic vRNA and suppression of spliced message was not prominent in BG LV. In wild type HIV virus, the presence of Rev/RRE is required along the entire mRNA transport and utilization pathway for the stabilization, correct subcellular localization, and efficient translation of RRE-containing mRNA. The data presented here confirms and extends a recent study that shows that RRE had a minor effect on cytoplasmic vRNA levels, but reduced viral titers approximately 100-fold. It further shows that Rev/RRE requirement is specific for large transgenes, but dispensable for small expression cassettes. Unlike a previous report in the literature, the present research did not see a role of RRE in vRNA stabilization, since equal or higher amounts of vRNA was seen with vectors without RRE. The likely mechanism is the capacity of RRE to be involved in viral assembly and packaging.

### Example 11

### Role of Env SA and Gag Sequences

Presence of the env SA has been shown to stabilize the viral genome, resulting in a higher virus production. Presence of SA may also stabilize the vRNA at a post-entry level, since some vectors without the env SA, when compared to analogous vectors with the env SA had the same v-RNA but had lower transduction/titer in target cells The gag sequence, with a start codon mutation to prevent the translation of the gag protein, helps the production of LV during viral packaging. In this study it was determined that this requirement was specific to large transgene cassettes. It was also demonstrated that removal of an inhibitory sequence present between 414 bp and 631 bp of the gag gene that has been previously shown to decrease the stability of gag-containing RNAs, increased titers by 3.5-fold.

In conclusion, this research describes the steps in the viral life-cycle affected by the non-coding cis-sequences when LV encodes large transgene cassettes; and their dispensability for smaller transgenes such as GFP. These results provide new insight in the design of LV vectors. Gutted/minimal LV could be designed for small therapeutic transgenes, which would be less recombinogenic and safer in gene therapy applications.

### Example 12

### Viral Vector Design

LV: To clone the sSIN-GFP vector, the 3′LTR of a standard SIN-LV backbone previously used was modified to improve transcript termination: 0-growth hormone polyadenylation signal was added downstream the 3′LTR and a USE sequence derived from SV40 late polyadenylation signal was added in the U3 deletion. The dsSIN-GFP was obtained by removing the ClaI-Nrul fragment from the sSIN-GFP plasmid. A multi-cloning site (MCS-ClaI-Eco47III, Xhol, Smal, Sall, EcoRI: CCATCGATAGCGCTCTCGAGCCCGGGGTCGACGAATTCC (SEQ ID NO: 1)) was cloned in the Clal and EcoRI sites of sSIN. The 0-globin-LCR (BG) cassette was cloned in reverse orientation into the Xhol and Smal sites and this parent construct was termed sSIN-BG. sBG-0 was obtained removing the region between Eco47III and Nrul, leaving behind only HIV-1 packaging sequence (w) following the 5′LTR from sSIN-BG. cPPT was cloned into sBG-0 Clal site (sBG-1). PCR fragments for RRE, RRE-env, short gag (360 bp), long gag (630 bp) were cloned in Xhol blunted site, and these vectors were termed sBG-2, sBG-3, sBG-10, sBG-9, respectively. Primers sequences, where F denotes forward primers and R denotes reverse primers:

RRE F:
(SEQ ID NO: 2)
ATAAACCCGGGAGCAGTGGGAATA;
RRE R:
(SEQ ID NO: 3)
ACATGATATCGCAAATGAGTTTTCC;
ENV R:
(SEQ ID NO: 4)
ACATGATATCATACCGTCGAGATCC;
GAG F:
(SEQ ID NO: 5)
ACTGCTCTCGAGCAATGGGAAAAAATTCGGT;
GAG 1R:
(SEQ ID NO: 6)
ACTGCTCTCGAGGCAGCTTCCTCATTGATG;
GAG 2R:
(SEQ ID NO: 7)
ACTGCTCTCGAGATCAGCGGCCGCTTGCTGT.

A frame-shift mutation was inserted in the 5′ sequence of gag in the start codon to disable the gag start site, using the primer Gag F that inserts the dinucleotide CA in the gag ATG. Vectors sBG-7 and sBG-8 were obtained cloning long gag and short gag PCR fragments into Xhol site of sBG-2. A point mutation to disrupt the SA site in the env sequence was performed using MutSAF (TATCGTTTCGAACCCACCTCC (SEQ ID NO: 8)) and MutSAR (GGAGGTGGGTTCGAAACGATA (SEQ ID NO: 9)) primers to generate sBG-4 (the wt SA sequence CAG inside the Env fragment was mutated into CGA). sBG-5 was obtained cloning the long gag PCR fragment into the Xhol site of sBG-3. γRV: SRS11.SF γRV plasmid was kindly provided by Drs. Axel Schambach and Christopher Baum, (Hannover, Germany). In SRS11.BG vector, the human β-globin-LCR (BG), was cloned in reverse orientation into the Pstl site of SRS11.SF retroviral vector plasmid. All vector cartoons are depicted in FIG. 2.

### Example 13

### Virus Production

LV was produced by transient co-transfection of 293T cells, as previously described using the vector plasmids, the packaging (A8.9) and the envelope (VSV-G) plasmids; virus-containing supernatant was collected at 60 hours after transfection and concentrated by ultracentrifugation. All vectors in an experiment were packaged simultaneously and the virus was concentrated 1400-fold from all viral supernatants by ultracentrifugation at 25,000 rpm. Viral titers were determined by infecting mouse erythroleukemia (MEL) cells or HT1080 cells with serial dilution of concentrated virus, differentiating them, and analyzing them for HbA or GFP expression by fluorescence-activated cell-sorter (FACS) as previously described. γRV were produced similarly but not concentrated. All transfections and subsequent titration were performed in triplicate. Packaging of vectors, with and without Rev, was performed following a similar method, except that the packaging plasmid 48.9 was replaced with pMDLg/pRRE and pRSV-Rev. The ratio of vector plasmid:pMDLg/pRRE:pRSV-Rev:VSV-G was 4:4:3:1.

### Example 14

### Cell Lines

Murine erythroleukemia cell (MEL) line and 293T cells were maintained in Dulbecco modified Eagle Medium (DMEM, Mediatech, Inc, Herndon, VA) supplemented with 10% heat inactivated fetal bovine serum (FBS) (U.S. Bio-technologies, Inc, Parker Ford, PA). MEL cells were induced to differentiate in DMEM containing 20% FBS and 5 mM N,N′-hexamethylene bisacetamide (Sigma), as previously described in the art.

### Example 15

### HbA Staining and FACS Analysis

The methodology used to label human β-globin using the anti-human HbA antibody was as previously described. Briefly, cells were fixed in 4% paraformaldehyde for 60 minutes at room temperature, washed once with phosphate-buffered saline (PBS), and the pellet resuspended in 100% methanol for 5 minutes. The fixed cells were then washed with PBS, and nonspecific antibody (Ab) binding was blocked using 5% nonfat dry milk for 10 minutes at room temperature. Subsequently, cells were washed in PBS, pelleted, and permeabilized. The cells were divided into 2 tubes and stained with either anti-zeta globin-fluorescein isothiocyanate (FITC) Ab (1 μg/10⁶ cells) as a negative control or anti-HbA-FITC Ab (0.1 μg/10⁶ cells) (Perkin Elmer, Waltham, MA) for 30 minutes at room temperature in the dark. Unbound Ab was removed by a final wash with PBS before they were analyzed on FACS Calibur (Becton Dickinson, Franklin Lakes, NJ).

### Example 16

### Total and Cytoplasmic RNA Northern Blot

293T cells were harvested and washed in PBS 72 hours after transfection. Isolation of nuclear and cytoplasmic RNA is obtained with a 7 minutes incubation on ice with NEB buffer (10 Mm Tris-HCl pH 7.4; 10 mM NaCl, 3 mM MgCl2; 5% IGEPAL). After centrifugation RNA-STAT (Tel-Test, INC, Texas) was added to the supernatant that contains cytoplasmic RNA, and proceeded with RNA extraction following manufacturer's instructions. Total RNA was extracted from 293T cells using RNA-STAT. Northern Blot was then performed according to standard protocol. The blot was hybridized with a 32P labeled β-globin probe. To normalize the loading of the RNA, membranes were then stripped and re-probed with a 32P labeled 18S probe. To test the purity of cytoplasmic RNA membranes were stripped and re-probed with a 32P labeled probe specific for GAPDH intron probe that detected no intronic transcript in the cytoplasmic preparation.

### Example 17

### Genomic Southern Blot

Genomic DNA was performed on DNA isolated from transduced MEL cells and 10 μg of genomic DNA was digested with Aflll enzyme and Southern Blot performed according to standard protocol. The blot was hybridized with a HS2 fragment of the β-globin LCR probe. RNA dot blot vRNA was extracted from same volumes of concentrated viruses using the QIAamp vRNA Mini Kit (Qiagen) following the manufacturer's instructions. Briefly the virus was lysed under highly denaturing conditions and then bound to a silica-gel-based membrane. Two washing steps efficiently washed away contaminants and vRNA was eluted in 30 μl of DEPC-water. After elution vRNA was treated for 20 min at room temperature with DNAse I, amplification grade DNase I (Invitrogen, Carlsbad, CA) was inactivated by incubating the sample at 65°. vRNA was then denatured in 3 vol of denaturation buffer (65% formamide, 8% formaldehyde, MOPS 1×) for 15 min at 65°. After denaturation 2 vol. of ice-cold 20×SSC were added and the RNA was bound to a nylon membrane by aspiration through a dot-blot apparatus. The blot was hybridized with a 32P labeled β-globin specific probe and an X-ray film was exposed overnight.

### Example 18

### Chromatin Insulators-Generally

Chromatin insulators separate active transcriptional domains and block the spread of heterochromatin in the genome. Studies on the chicken hypersensitive site-4 (cHS4) element, a prototypic insulator, have identified CTCF and USF-1/2 motifs in the proximal 250 bp of cHS4, termed the “core”, which provide enhancer blocking activity and reduce position effects. However, the core alone does not insulate viral vectors effectively. The full-length cHS4 has excellent insulating properties, but its large size severely compromises vector titers. A structure-function analysis of cHS4 flanking lentivirus-vectors was performed and transgene expression in the clonal progeny of hematopoietic stem cells and epigenetic changes in cHS4 and the transgene promoter were analyzed. The core only reduced the clonal variegation in expression. Unique insulator activity resided in the distal 400 bp cHS4 sequences, which when combined with the core, restored full insulator activity and open chromatin marks over the transgene promoter and the insulator. These data consolidate the known insulating activity of the canonical 5′ core with a novel 3′ 400 bp element with properties similar to the core. Together, they have excellent insulating properties and viral titers. This data has important implications with respect to understanding the molecular basis of insulator function and design of gene therapy vectors.

### Example 19

### Vector Constructs and Experimental Design

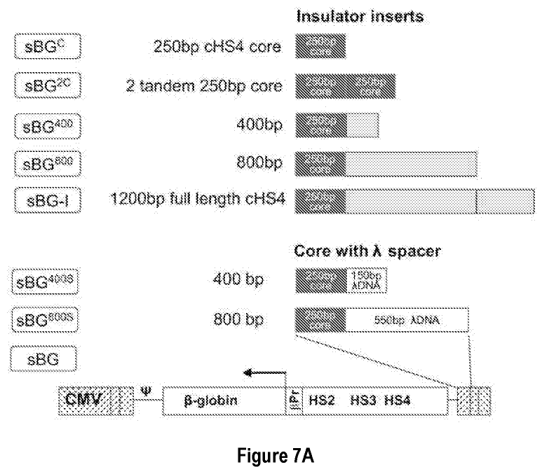

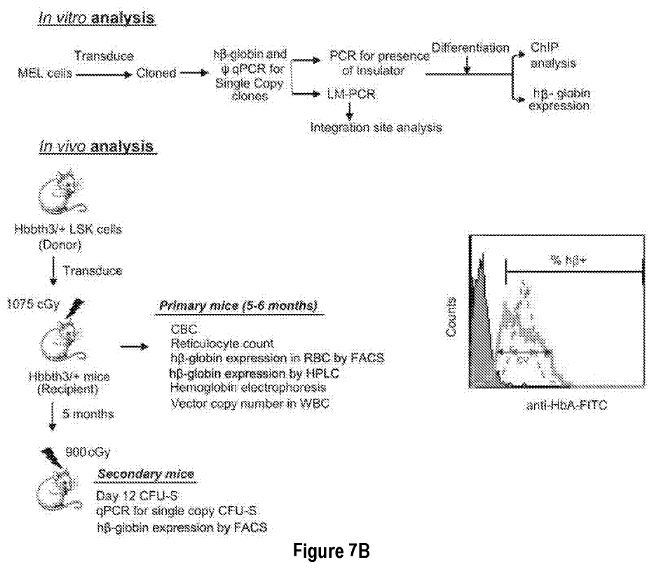

Self-inactivating lentivirus vectors were designed to incorporate either the 5′ 250 bp “core” (sBGC), two tandem repeats of the core (sBG2C), 5′ 400 bp (sBG400), 5′ 800 bp (sBG800) or the full-length 1.2 Kb cHS4 insulator (sBG-I). All vectors carried the human (h) β-globin gene and promoter and the locus control region enhancer. The different insulator fragments were cloned in the forward orientation into the U3 region of 3′ LTR, so that upon reverse transcription, integrated provirus in target cells has the insulated 3′ LTR copied to the 5′LTR, and flanks the hβ-globin expression cassette at both ends. To assess whether elements outside the 5′ 250 bp core merely provided a spatial scaffold, vectors with inert DNA spacers downstream of the core, sBG400S and sBG800S, were also tested. All vectors were compared to the uninsulated control, sBG (FIG. 7A).

First, MEL cells were infected with each of the lentivirus vectors and single integrant MEL clones were identified (FIG. 7B). All analysis was performed only on single-copy MEL clones that carried hβ-globin and verified to have intact insulator sequences by PCR, and subjected to qPCR for vector copy number; hβ-globin expression was analyzed by FACS: 1) the percentage of hβ-globin expressing cells (% hβ+ cells) was used to determine chromosomal position effects, and 2) the variation of expression of hβ-globin expression in cells within a clone, as determined by the coefficient of variation (CV), was used to determine the clonal variegation in expression (FIGS. 7A and 7B). CUP analysis was performed on the histones over the insulator regions and hβ-globin gene promoter in the different proviruses to study epigenetic modifications. Chromatin position effects of these vectors were confirmed in vivo, in RBC of Hbbth3/+ thalassemia mice transplanted with vector-transduced HSCs 24 weeks after transplant. Secondary transplants were then performed and single-integrant CFU-S following transplants were analyzed for hβ-globin protein and mRNA. In mice, hematological analysis, and HPLC forh0-globin protein were additionally performed to quantify expression.

### Example 20

Regions of cHS4 Necessary to Protect from Chromatin Position Effects

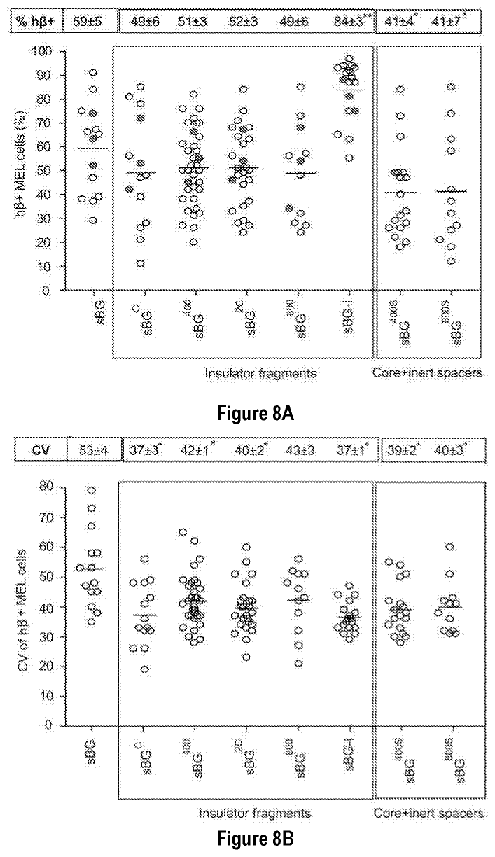

Consistent with previous results, a very high % of hβ+ cells were present in the sBG-I single-integrant clones compared to control sBG clones (P<0.01); the % of hβ+ cells in sBGC, sBG2C, sBG400 and sBG800 clones were not significantly different from the sBG control clones (FIG. 8A) In order to ensure that the presence of cHS4 in the LTR did not bias integration, and that the analysis was performed on distinct clones, by LM PCR and integration site sequencing on ten randomly selected sBG or sBG-I MEL clones. Insertions occurred near/in distinct genes between uninsulated and insulated clones, with no apparent bias. The presence of the cHS4 core (sBGC), or extended sequences of the insulator downstream to the core, up to 800 bp, did not increase the % hβ+ cells further; neither did tandem repeats of the core sequence, even though the latter has been shown to confer enhancer blocking effect in plasmid-based systems.

Another phenomenon seen with transgene expression is clonal variegation, defined as varying levels of expression in daughter cells with the same integration site. A quantitative way to determine clonal variegation is by FACS analysis of transduced clones and calculation of the coefficient of variation (CV) of expression of the transgene around the average expression of the transgene in the clone. The CV is a unit-less measure of variability calculated as ratio between sample standard deviation (SD) and the sample average. A high CV was observed in the uninsulated sBG clones (FIG. 2). The CV was significantly reduced in all vectors that contained the 5′ 250 bp core. These results were confirmed in clones derived from vectors that carried inert DNA spacers downstream of the core: sBG400S and sBG800S, showing that the reduction in CV was specific to the insulator core, and in contrast to the data on % of hf3+ cells, which required the full-length insulator to be present.

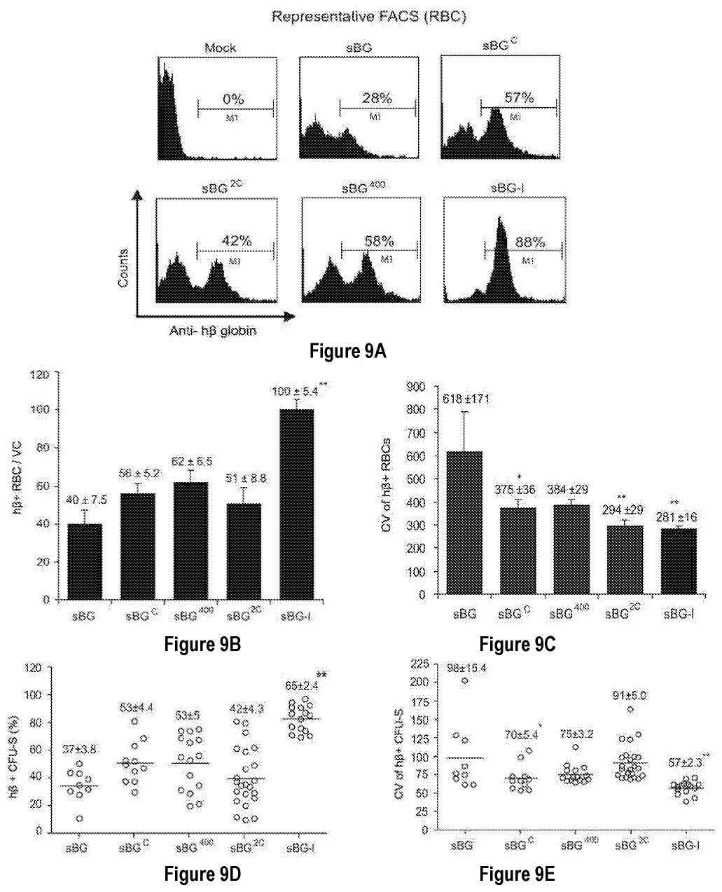

It was notable that PCR for insulator sequences showed absence of the insulator sequences only in sBG2C proviruses, with 6 of 24 clones (25%) MEL clones having both copies of the core deleted from both LTRs. There was no observed deletion of the insulator sequences in clones from all other vectors. Southern blot analysis of sBG2C MEL pools confirmed deletion of one/both copies of the core in the majority of cells. Reverse transcription of repeat sequences, known to result in recombination events in retroviral vectors likely caused unstable transmission of the vector with repeat core sequences. This effect of the core versus the full-length cHS4 was confirmed in vivo, in thalassemia mice. Peripheral blood RBC were analyzed for hβ-globin expression 6 months following transplant. FACS analysis in RBC from sBG, sBGC, sBG2C, sBG400 and sBG-I groups of mice (representative plots shown in FIG. 9A) shows that the % hf3+ RBC were significantly higher only in the sBG-I group of mice, compared to sBG group of mice, like the data in MEL cells; and the CV was significantly lower in all vectors that carried the core (P<0.01; FIG. 9B-C). Taken together, this data indicates that the full-length cHS4 is required to shield against chromosomal position effects.

### Example 21

### Chromatin Position Effects in the Clonal Progeny of Murine HSC Following Secondary Transplants

The chromatin position effects were next confirmed in single copy secondary CFU-S. The secondary colony forming units-spleen (CFU-S) assay is considered the most stringent assay that is a ‘gold-standard’ for studying epigenetic effects of chromatin insulator elements in cells derived from hematopoietic stem cells. Notably, no transduced CFU-S that was positive by PCR for vector-specific sequences that did not express hp-globin by FACS were observed, consistent with results reported on lack of transgene silencing with erythroid-specific SIN lentivirus vectors. FACS analysis for (1) % hβ+ cells and (2) TER-119 positive erythroblasts showed no difference in the percentage of TER-119+ cells between different vector groups (not shown). However, significantly higher % of hβ+ cells were only present in secondary CFU-S with the sBG-I vector. Again, the CV was significantly lower in CFU-S transduced with all the vectors carrying the core, compared to uninsulated sBG transduced CFU-S (FIGS. 3A and 3B). Real-time RT-PCR analysis on six randomly selected CFU-S from each group of mice showed that compared to the sBG vector, mRNA expression from the sBG-I CFU-S was approximately 2-fold higher. However, expression from sBGC, sBG2C and sBG400 transduced CFU-S was not significantly different from that of sBG CFU-S. Taken together, these data indicate that the 5′ 250 bp core sequences in sBGC, sBG400, sBG400S, sBG800 and sBG800S specifically reduced the clonal variegation of hβ-globin expression. However, the full-length cHS4 element was required for improved probability of expression from different integration events.

### Example 22

Patterns of Histone Acetylation and Methylation in the Core Region and the/3-Globin Promoter Region in Insulated Vectors

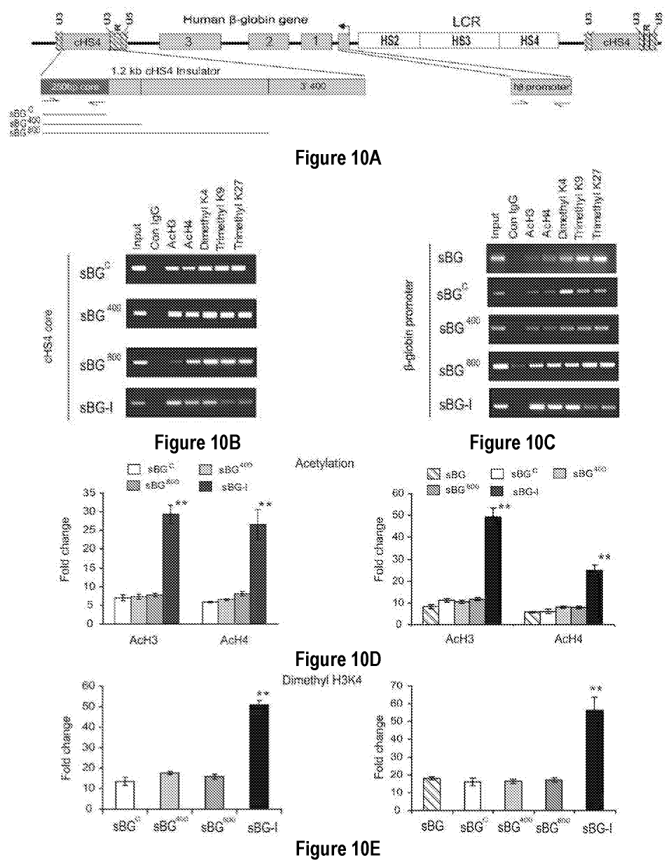

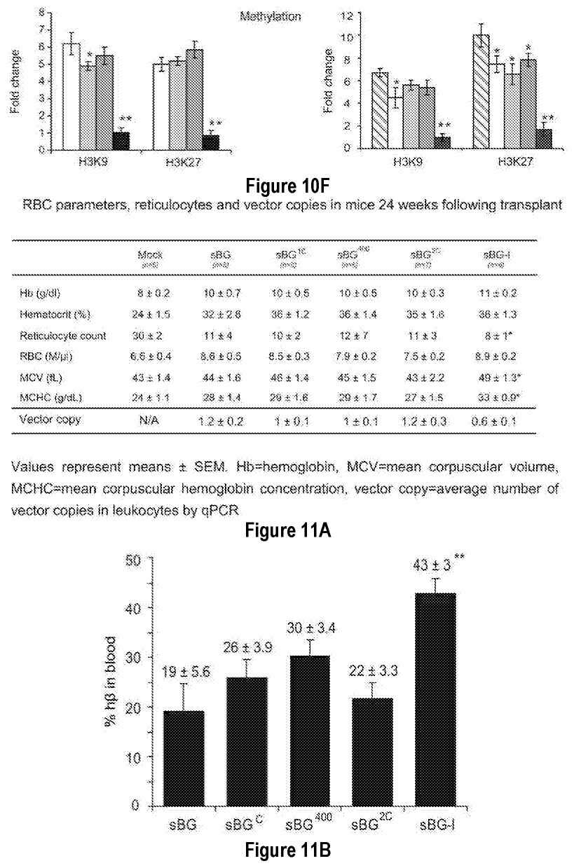

Next the epigenetic modifications that accompany the specific effects seen with the various insulator regions were determined by comparing the relative levels of active histone marks acH3, acH4 and H3K4me2 and repressive histone marksH3K9me3 and H3K27me3 between different proviruses in MEL clones. ChIP analysis was performed on the cHS4 core in three representative clones that were pooled together for each vector (clones chosen are shown as filled circles in FIG. 8A) by semi-quantitative PCR (FIG. 10B-C) and real-time PCR) (FIG. 10D-F). Clones carrying the sBG-I vector integrants showed approximately 6-fold enrichment of the active chromatin marks and decreased repressive chromatin marks over the cHS4 “core” fragment, compared to sBGC, sBG400 and sBG800, three vectors that carried the “core”.

Histone modifications were analyzed over the hβ-globin promoter in the uninsulated vector (sBG) and all other vectors, which carried the “core”, to assess whether differences in histone patterns over the transgene promoter in vectors may have contributed to the reduced clonal variegation. There was a small but significant reduction in repressive chromatin patterns H3K27me3 with sBGC, sBG400 and sBG800 proviruses, compared to the uninsulated sBG provirus (FIG. 10F, right panel). However, with the sBG-I provirus, where maximal insulator activity was present, the hβ-globin promoter region had markedly reduced repressive chromatin patterns.

These data show that the “core” sequences and extension of the core up to the 5′ 800 bp of cHS4 reduced activation marks over the transgene promoter to a small extent. However, a major reduction in repressed histone modifications over cHS4 and the transgene promoter region only occurred when the distal 3′ 400 bp sequences of cHS4 were present in addition.

### Example 23

Hematological Parameters in Thalassemia Mice Transplanted with HSCs Transduced with Uninsulated and Insulated Vectors

The anemia, reticulocytosis and other RBC indices were improved even with the sBG vector (FIG. 11A), consistent with published reports with uninsulated hβ-globin lentivirus vectors. Hemoglobin of mock-transplanted mice was 7.7±0.2 gm/dL and the sBG group of mice was 10.4±0.7, with 1.2 vector copy per cell. It was noteworthy that the sBG-I group of mice had higher hemoglobin and the lowest reticulocyte count, despite having half the vector copies per cell compared to the sBG group of mice (hemoglobin 11±0.2 gm/dL; 0.6 vector copies per cell). When normalized for transduction efficiency, this amounts to a 5.2 gm increase in hemoglobin per vector copy in sBG-I mice over mock mice, in contrast to a 2.3 gm increase in hemoglobin per vector copy in the sBG mice. RBC parameters from the experimental mice showed significant improvement (FIG. 11A; note that these data are not normalized for number of vector copies). Improvement in these indices was highest with the sBG-I mice, albeit not significantly different unless normalized for vector copy.

HPLC analysis for hβ-globin protein in blood confirmed significantly higher hβ-globin expression only in the sBG-I mice: 43±3% of the total hemoglobin in RBC was derived from hp-globin (hβ2mα2) in sBG-I mice as compared to 19±6% in the sBG mice, while that in sBGC, sBG400 and sBG2C group of mice was not significantly different from control (FIG. 11B). Human hβ-globin expression and hematological parameters in the sBG2C group of mice were similar those seen in the uninsulated control group.

### Example 24

Insulator Activity in the 3′400 cHS4 Region

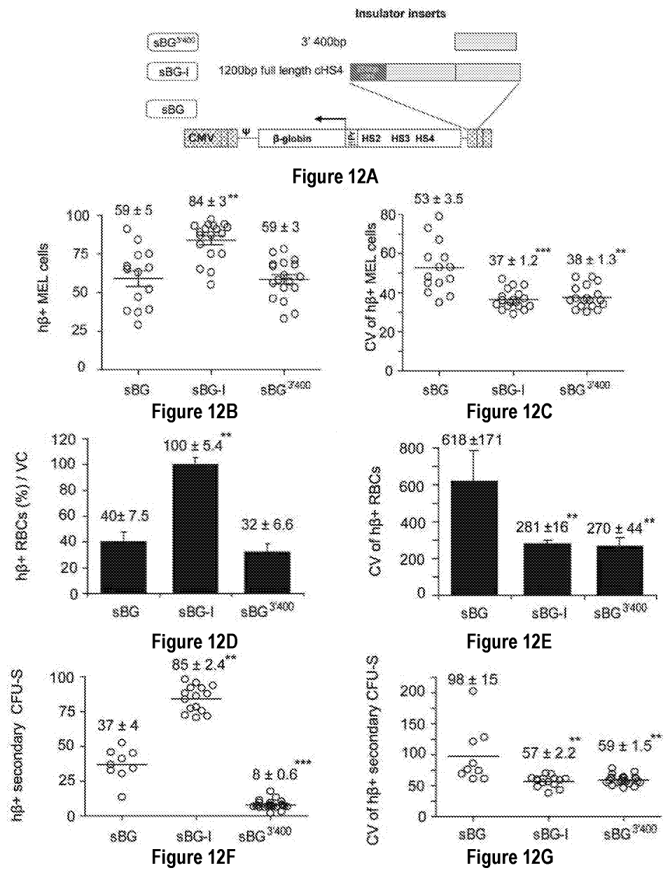

Since the 5′ 800 bp of cHS4 only reduced the CV, while full insulator activity was restored with the full-length 1.2 Kb insulator. A vector was generated carrying only the distal/3′ 400 bp region of the cHS4 (sBG3′400) derived MEL clones and mice were transplanted with sBG3′400-transduced LSK cells. Note that unlike vectors described earlier, this vector does not contain the 5′250 bp “core” sequences (FIG. 12A). The sBG3′400 vector had no effect on % of hβ+ cells in MEL clones or the % hβ+ RBC in mice (FIG. 6B,D), an effect comparable to sBG clones, or those carrying the 5′ 250 bp “core” (sBGC). However, like all vectors carrying the 5′ core, sBG3′400 significantly reduced the CV of hp-globin expression in MEL clones and in RBC (FIG. 12C,E).

The amount of hβ-globin protein in the sBG3′400 mice, determined by HPLC analysis, was not significantly different from sBG (17.5±3% versus 19.5±5.6%), but was at least 2-fold lower than that seen in the sBG-I mice (43±3%; P<0.01) (FIG. 12F). Overall, the 3′ 400 bp of cHS4 had activity that was very similar to the 5′ 250 bp core (FIG. 9): it reduced clonal variegation, reflected in a reduced CV of hβ-globin expression in MEL clones and in RBC, but had no effect on the proportion of hp-globin expressing red cells. “Core-like” effects of the 3′ 400 bp in individual single copy secondary CFU-S (FIG. 12G), were confirmed, with results similar to those with the sBGC vector (FIG. 9D-E). The 3′ 400 region has no known consensus sequences for CTCF or USF-1, and this region has not been previously analyzed. It was noteworthy that neither the 5′ core, nor the 3′ 400 bp, when present alone, were able to improve the probability of expression of integrants/protect from position effects.

### Example 25

Insulator Activity of the 5′ “Core” Combined with the 3′ 400 bp

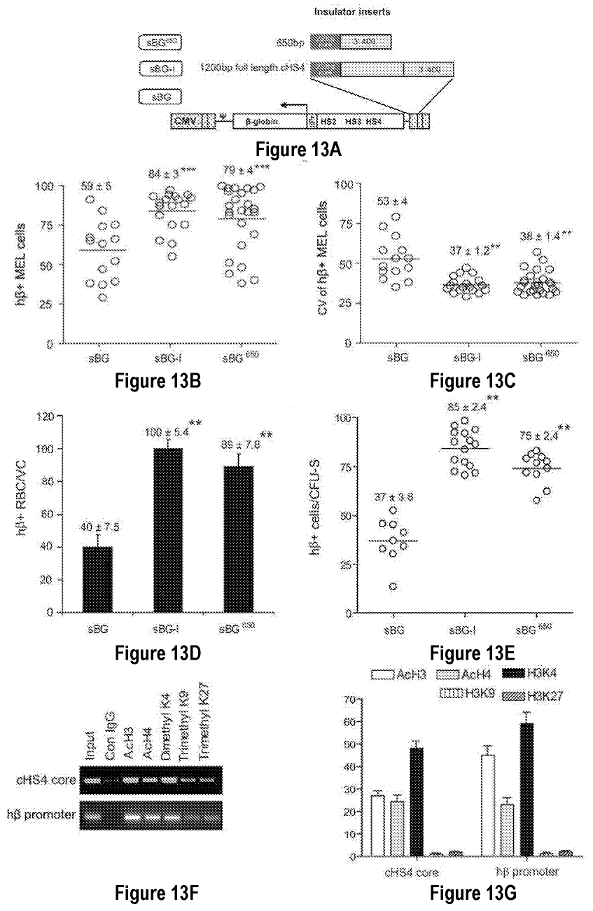

When the 5′ 250 bp core and the 3′ 400 bp sequences of cHS4 insulator (sBG650 vector; FIG. 13A) were combined, this vector performed similarly to the sBG-I vector—in MEL clones, in RBCs of transplanted mice and in secondary CFU-S. The proportion of hβ-globin expressing cells in sBG650 MEL clones and RBC (FIG. 13B-D) was significantly higher compared to sBG clones (P<0.001), and was similar to sBG-I clones. Likewise, the CV of the sBG650 clones was comparable to sBG-I clones (FIG. 13C). The hβ-globin expression in the RBC of primary mice was comparable to sBG-I mice (FIG. 13D). The amount of hβ-globin protein in the sBG650 mice, determined by HPLC analysis, was not significantly different from sBG-I mice (41±2.6% versus 43±3%, respectively), but was at least 2-fold higher than that seen in the sBG mice (19±6%; P<0.01). Five months after transplant, secondary transplants were performed to generate CFU-S, which confirmed that the sBG650 vector restored insulator activity similar to that seen with sBG-I vector (FIG. 13E). The chromatin configuration over the core in sBG650 proviruses (FIG. 13F) showed restoration of open chromatin patterns both over the insulator core and the β-globin promoter, identical to those seen in the sBG-I proviruses (FIG. 10).

### Example 26

Epigenetic Modifications in the 3′400 bp Region of cHS4 and its Interaction with the Core

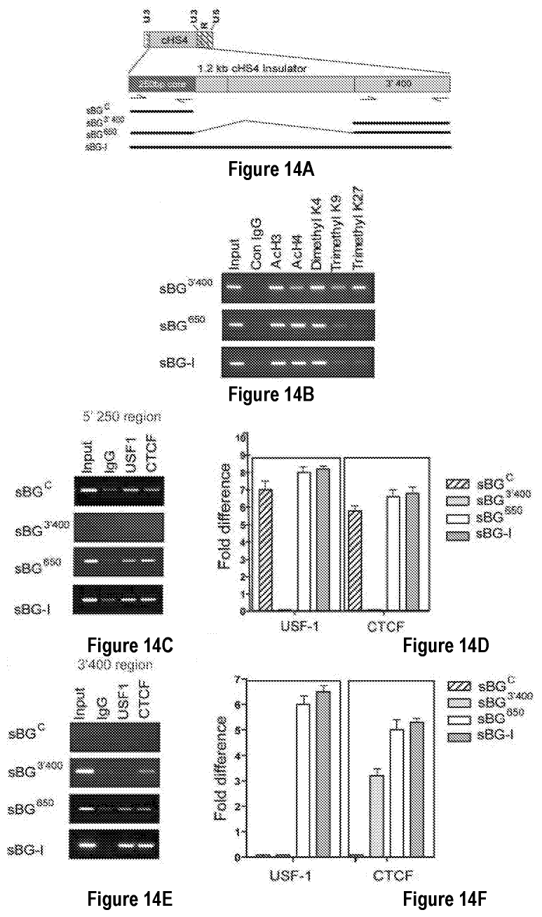

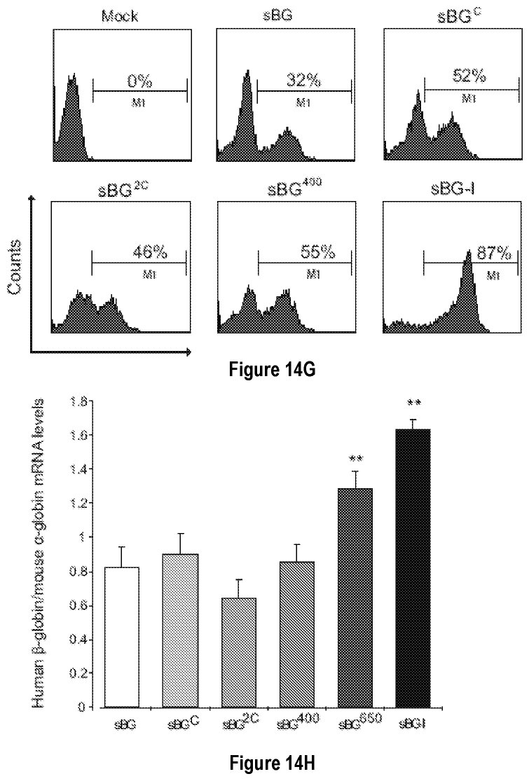

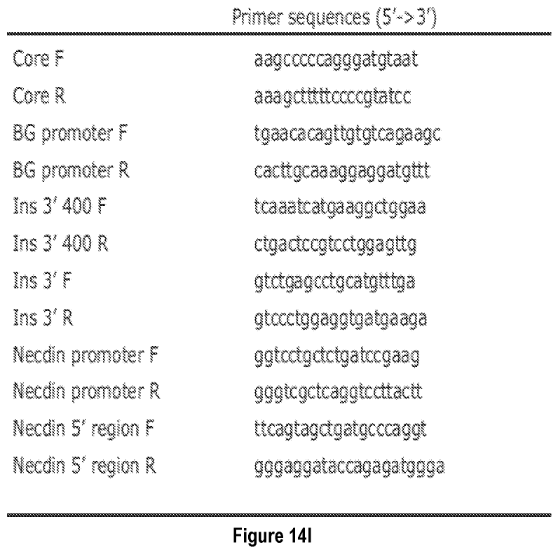

The chromatin configuration of the distal 3′ 400 bp portion of cHS4 have not been previously studied. The histone patterns were first analyzed over the 3′ 400 bp region (sBG3′400) when present alone (sBG3′400), or when in combination with the 5′ core (in sBG650 and sBG-I) (FIG. 14). The acetylation and methylation patterns of the histones in the 3′400 region of sBG3′400 provirus (FIG. 14B) were similar to those seen in the 250 bp core region in the sBGC provirus (FIG. 10). However, in sBG650 and sBG-I proviruses, the 3′ 400 bp sequences had increased acetylation marks and reduced repressive, showing once again, that the combination of the proximal and distal ends of cHS4 is necessary for open chromatin patterns. This effect was reminiscent of the ChIP analysis over the 5′ core region or the β-globin promoter region in sBG-I (FIGS. 10D and F) or sBG650 (FIGS. 13F and G). Taken together, the genetic and epigenetic analysis indicated that the 5′ and 3′ ends of the insulator were functioning as two cores, which interacted for epigenetic modifications of chromatin on the insulator and promoter, to impart adequate insulator activity.

The 3′ 400 bp region, however, has no known CTCF or USF-1 motifs, that have been shown to impart enhancer blocking and barrier activity, respectively, to cHS4. It is conceivable; however that CTCF and/or USF-1 may perhaps be recruited to the 3′400 region. Using antibodies to USF-1 and CTCF, chromatin was immunoprecipitated from sBGC, sBG3′400, sBG650 and sBG-I proviruses from MEL clones. ChIP analysis was performed using semi-quantitative PCR and qPCR. When primers to the core region were used to amplify ChIP products, CTCF and USF-1 recruitment to the 5′ core region was evident (FIG. 14C-D), as anticipated and shown previously. Interestingly, when 3′400 region primers were used to amplify the ChIP products, the sBG3′400 provirus showed enrichment for CTCF, albeit at somewhat lower levels than that seen over the core region. More notably, however, the sBG650 and sBG-I proviruses showed enrichment both USF-1 at the 3′ 400 bp region, an effect seen when both the proximal core and the distal 400 bp sequences were present. The 3′ 400 bp region, when present alone in sBG3′400, did not bind USF-1 (FIG. 14E-F). These data indicate that the 3′ 400 bp region interacts with CTCF despite lack of the CCCTC consensus, which may explain the “core-like” activity in this region and the interaction between the 5′ core region and the 3′ 400 region of the cHS4 insulator (in sBG-I or sBG650) likely occurs via USF-1.

### Example 27

Vector Titers with the 650 bp cHS4 Insulator

The 1.2 Kb cHS4 remarkably lowers titers of SIN-lentivirus vectors, limiting large-scale virus production for human trials. It has been recently shown that the mechanism of reduction in titers is specifically due to the length of the insert in the 3 ′LTR. Compared to sBG, sBG650 had very reasonable titers that were only 2.5±0.9 fold lower than sBG, in contrast to 10.4±2 fold lower titers of sBG-I (n=3). Therefore, this optimized insulator can be used for the design of safer gene therapy vectors which would provide uniform and therefore higher expression and be scalable to large-scale production.

The full-length cHS4 insulator has been previously shown by us and by others to protect viral vectors against chromosomal position effects. The profound deleterious effects on viral titers however, have precluded its utility. Attempts to use only the 5′ 250 bp of cHS4, characterized to be the core of the insulator, have failed in viral vectors despite significant activity of the core in plasmid based systems, and loss of insulator activity with mutations in these regions.

Regions surrounding the cHS4 insulator and β-globin promoter have been shown to constitutively higher marks of active chromatin in the native location. The cHS4 prevents the spread of heterochromatin to the β-globin domain, even when adjacent heterochromatin domains have high repressive histone marks, H3K9me3 and H3K27me3. Clones carrying the sBG-I vector integrants showed an enrichment of the active chromatin marks and a striking decrease in repressive chromatin marks over the cHS4 core compared to sBGC, sBG400 and sBG800 vectors, where no significant differences in these epigenetic marks were observed.

Mechanistically, the USF-1/2 element in the insulator has been shown to recruit histone modifying enzymes to the core, and interact with histone lysine methyl transferase SETT/9 and p300/CREB-binding protein-associated factor (PCAF), thus increasing active chromatin marks. However, No such increase was observed in acH3, acH4 and H3K4me2 over the core or the 3′ 400 bp when they flanked the transgene in the sBGC, sBG400, sBG800 and sBG3′400 vectors. This effect required the vector carrying the full length cHS4 (sBG-I, FIGS. 10 and 14) or both the core and 3′400 bp combined sBG650 vector (FIGS. 13 and 14). CUP analysis over the hβ-globin promoter showed that compared to an uninsulated vector, the core alone reduced repressive chromatin marks over the promoter to some extent (FIG. 10F), which may account for the reduction in CV from vectors carrying the core. However, the core was dependent on the 3′ 400 bp region and conversely, the 3′ 400 bp region dependent on the core for the high degree of histone acetylation and absent to minimal repressive marks over both these regions.

Models proposed to explain the effect of the cHS4 on surrounding chromatin include protection against transgene silencing by exclusion of methyl-CpG-binding proteins; indeed, cHS4 has been shown to block silencing by retroviral vectors. No extinction of β-globin expression over time was observed, even with the uninsulated vector in mice, or MEL clones maintained up to 6 months in culture (data not shown) This may be due to several USF-1 elements in the β-globin LCR hypersensitive sites, that have been shown to interact with the E-box elements located in HS2 and in the β-globin gene promoter. It is conceivable that this resistance to silencing conferred by the LCR may override any activity seen with the cHS4 core. These results contrast those by Panell et al that retroviruses including those derived from HIV-1, dominantly silence a linked locus control region (LCR) beta-globin reporter gene in transgenic mice. Methylation was analyzed and it was subsequently reported that there was a lack of CpG methylation and extinction in expression with erythroid-specific SIN-lentivirus vectors in vivo, in primary and secondary recipients. This data suggests that in erythroid vectors, which otherwise resist silencing via promoter methylation, the full-length cHS4 was able to modify the histone patterns over the transgene promoter, and over itself to reduce position effects.

Intriguingly, the in silico analysis of the 3′ 400 bp region revealed no CTCF or USF1 binding sites, but sites for multiple known transcription factors. Any of these transcription factors, or perhaps a novel protein may be the interacting partner with the CTCF and/or USF-1. CTCF directly regulates the balance between active and repressive chromatin marks via binding to the cohesin complex. This data reveals that the 3′ 400 bp region can also interact with CTCF: although co-immunoprecipitate the 3′400 bp and CTCF from the sBG3′400 provirus (FIG. 14C-F) was unsuccessful.

Interestingly, the 3′400 bp co-immunoprecipitated with USF-1 antibody only when the 5′ core sequences were additionally present, suggesting that USF-1 likely forms a bridge between the 5′ and 3′ end of cHS4 to reduce position effects. Whether elements within the 3′ 400 bp recruit histone acetylases that bind USF-1 or cohesin and/or nucleophosphmin complexes to affect position effects would be important to determine.

Ultimately, a systematic genetic and epigenetic analysis of insulator activity of the cHS4 in vitro and in vivo was performed and novel “core-like” activity in the 3′ 400 bp was identified. The 3′ 400 bp of cHS4, which contains no consensus sites for USF or CTCF, nevertheless binds CTCF, while USF-1 appears to bind and bridge the 5′ core and the 3′ 400 bp of cHS4. New vector systems flanked by the optimized 650 bp′ cHS4 sequence, can provide excellent insulation of the transgene without significant loss in viral titers and have important safety and efficacy implications for gene therapy.

### Example 28

### Materials and Methods-Lentivirus Vectors

All vectors were obtained by cloning the different insulator fragments into Nhel/EcoRV sites in the U3 3′LTR region of the lentivirus plasmid, as described. This plasmid carried the human (h) β-globin gene and its regulatory elements (BG). All insulator fragments were amplified by PCR using the insulator plasmid pJCI3-1 (kindly provided by Dr. Gary Felsenfeld, NIH, MD) and verified by sequencing, as described. Cloning of the hβ-globin vector with and without the 1.2 kb cHS4 insulator has been described previously. The sBGIC vector was cloned by inserting EcoRI/XbaI 250 bp core insulator PCR product into sBG into BamHI/EcoRI restriction sites of the pBS plasmid. A second copy of the 250 bp core was then added into the pBS 1-core plasmid into EcoRI/KpnI sites, thus obtaining the pBS 2-core plasmid. The two tandem copies of the 250 bp core were then isolated digesting the pBS-2 core plasmid with KpnI/XbaI, and then cloned into the sBG vector, obtaining sBG2C. The sBG400 and sBG800 vectors were obtained by cloning the 2 PCR products into the sBG Nhel/EcoRV sites. The vectors containing DNA spacers were obtained amplifying different sizes of k-phage DNA using the following primer combinations: spacerF1 and spacerR1, spacerF1 and spacerR2, amplifying 150 bp, 550 bp k-DNA, respectively. Clal/EcoRI digested PCR fragments were ligated into EcoRI/ClaI sites in the pBS-1 core plasmid, and 400 bp and 800 bp fragments from the pBS-1 core plasmid were restricted with Hincll/Xbal and XbaI/Xhol, respectively, and cloned into Nhel/EcoRV sites of sBG. Virus was produced by transient co-transfection of 293T cells and titrated on MEL cells.

### Example 29

### Materials and Methods-Cell Lines

MEL cells and 293T cells were maintained in DMEM (Mediatech, Inc) supplemented with 10% heat-inactivated fetal bovine serum (FBS; U.S. Bio-technologies, Inc.) and differentiated as described. MEL cells were transduced to achieve less than 5% transduction efficiency for each of the vectors tested and cloned. Approximately 400 clones, derived from three independent transductions from each vector were screened by PCR for hβ-globin gene; positive clones were screened for an intact insulator region. Clones thus identified were then subjected to qPCR for single integrants, expanded and cryopreserved. An entire set of clones was thawed, differentiated and analyzed concurrently by FACS.

### Example 30

### Materials and Methods-Murine Hematopoietic Stem Cell Transduction and Transplants

Hbbth3/+ thalassemia mice were used for transplants. All animal studies were done using protocols approved by the Institutional Animal Use and Care Committee. Enrichment of lineage-Sca-1+c-kit+ (LSK) hematopoietic stem/progenitor cells was performed on single cell suspension of bone marrow by immunomagnetic separation and FACS sorting (details in supplementary Materials and Methods S1) LSK cells were transduced in Stem Span (Stem Cell Technologies Inc, Vancouver, BC) with concentrated vector supernatants at an MOI of 10, twice at 12 h intervals as previously described. 10,000 transduced LSK cells were co-transplanted with 2×105 LK cells into 10.75Gy irradiated thalassemia recipients. CFU-S assay: Discrete spleen colony forming units (CFU-S) were dissected at day 12 after transplant of bone marrow cells from primary mice 24 wk after transplant, as described earlier.

### Example 31

### Materials and Methods-Analysis for 143-Globin Expression

Complete blood counts were performed on a Hemavet (Drew Scientific, Inc, Oxford, CT, USA). Reticulocyte count was analyzed by staining 10 of whole blood with 200 μl of Retic-COUNT reagent (BD Biosciences, CA) and enumerated on the FACSCalibur (BD). Quantitative analysis of hβ-globin protein in RBC was performed on hemolysates of blood by high performance liquid chromatography (HPLC), as previously described and mRNA analysis quantified by real-time RT-PCR using validated primers and probes specific to hβ-globin (ABI Biosystems) using murine a-globin for normalization. FACS analysis following intracellular staining for hβ-globin was done as described before.

### Example 32

### Materials and Methods-Chromatin Immunoprecipitation (ChIP)

ChIP analysis was performed on MEL clones as described with minor modifications. Briefly, DNA samples from input and antibody-bound chromatin fraction were analyzed by qPCR using SYBR green (Applied Biosystems) using primer sets in triplicate, and data analyzed as previously described. The enrichment ratio was determined by calculating the ratio of DNA-ChIP to DNA-input and histone modification data normalized to the “no antibody” (IgG) control and primers corresponding to the necdin 5′ region and promoter region, as controls for repressed chromatin, to normalize the efficiency of immunoprecipitation. All the DNA-ChIP to DNA-input ratios were calculated as: 2 [Ct (Input)-Ct (ChIP)] divided with [dilution rate (ChIP)/dilution rate (Input)]. Ct values of all PCR products were determined by the SDS 1.2 software (Applied Biosystems). Mean and SEM values were determined for the fold difference, and two-tailed paired t tests to determine statistical significance (p<0.05).

### Example 33

Materials and Methods-Integration site analysis

Ligation-mediated (LM) polymerase chain reaction was performed as described by Modlich et al to map integration sites using primers and conditions described (Arumugam, Mol Ther 2009, in press citation).

### Example 34

### Materials and Methods-Statistical Analysis

Vectors were compared to the sBG vector Student's “t” test (unpaired and two tailed). ANOVA (Dunnett multiple comparison test) was also performed between groups for multiple comparisons. Data was expressed as mean±SEM. P<0.05 was considered significant.

### Example 35

Self-Inactivating Lentiviruses Flanked by the 1.2 Kb Chicken Hypersensitive Site-4 Insulator Element (cHS4) Provide Consistent, Improved Expression of Transgenes, but have Significantly Lower Titers

Self-inactivating lentiviruses flanked by the 1.2 Kb chicken hypersensitive site-4 insulator element (cHS4) provide consistent, improved expression of transgenes, but have significantly lower titers. Lengthening the lentivirus transgene cassette by an additional 1.2 Kb by an internal cassette caused no further reduction in titers. However, when cHS4 sequences or inert DNA spacers of increasing size were placed in the 3′LTR, infectious titers decreased proportional to the length of the insert. The stage of vector life-cycle affected by vectors carrying the large cHS4 3′LTR insert was compared to a control vector: There was no increase in read-through transcription with insertion of the 1.2 Kb cHS4 in the 3′LTR. Equal amount of full-length viral mRNA was produced in packaging cells and viral assembly/packaging was unaffected, resulting in comparable amounts of intact virus particles produced by either vectors. However, lentiviruses carrying cHS4 in the 3′LTR were inefficiently processed following target-cell entry, with reduced reverse transcription and integration efficiency, and hence lower transduction titers. Therefore, vectors with large insertions in the 3′LTR are transcribed and packaged efficiently, but the LTR insert hinders viral-RNA processing and transduction of target cells. These studies have important implications in design of integrating vectors.

### Example 36

Increased Length of the Vector Genome by 1.2 Kb does not Affect Viral Titers

One objective of the study was to determine if reduction in titers by cHS4 was secondary to additional lengthening of the viral genomes in the otherwise large hp-LCR (BG) lentivirus vector. Large viral RNA genomes are known to be packaged less efficiently in integrating vectors. Replication competent gamma-retroviruses delete added sequences and recombine to revert back to their original viral size. In gamma-retrovirus vectors that exceed the natural size of the virus, reduction in titers occurs at multiple steps of the viral life cycle-generation of full length genome, viral encapsidation/release and post-entry recombination events. Notably, BG lentiviruses contain transgene inserts of ˜7 Kb, and therefore do not produce viral-RNA genomes larger than the natural size/packaging capacity of the wild type HIV-1 virus. In lentivirus vectors, however, lowering of viral titers from transgene inserts 6 Kb or larger has been shown to occur from reduced packaging efficiency.

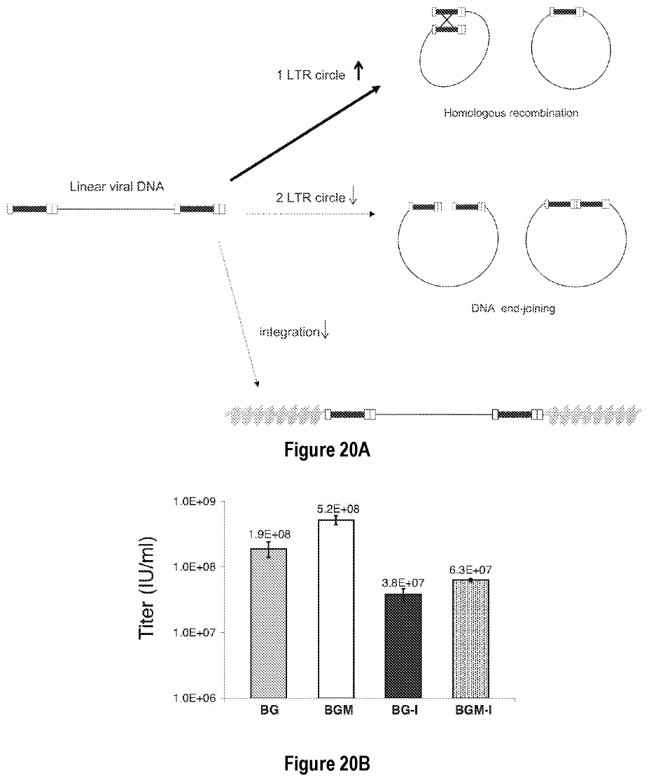

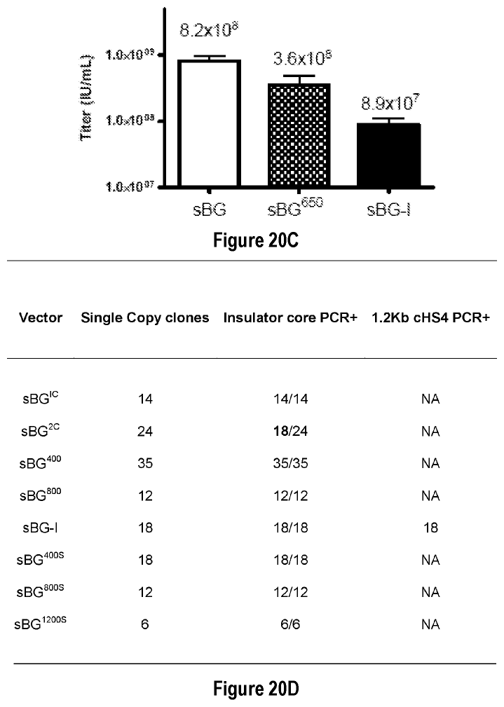

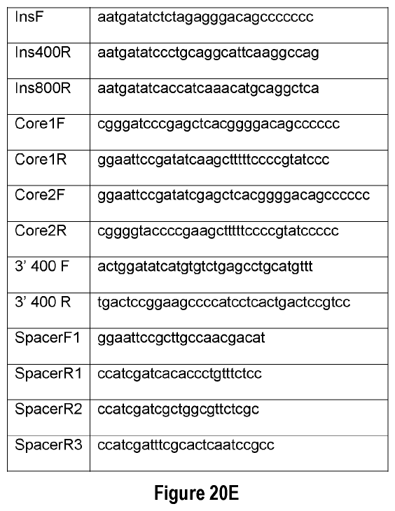

Uninsulated vectors BG and BGM were recently compared with analogous insulated vectors BG-I and BGM-I for position effects. The BG lentivirus vector carries the hβ and LCR, while a similar vector BGM additionally carries a PGK promoter driven methylguanine methyl transferase (P140K) cDNA (PGK-MGMT) insert downstream of the hp-LCR. The PGK-MGMT cassette is 1.2 Kb in size. The BG-I and BGM-I vectors carry the 1.2 Kb cHS4 insulator in the 3′LTR in addition. Virus was produced and processed identically from all four vectors and infectious titers were determined, as previously described. The titers of the concentrated BG vector were 2±0.5×10⁸ IU/mL, while that of BGM, carrying an additional 1.2 Kb internal cassette were slightly higher at 5±0.8×10⁸ IU/mL (n=4). In contrast, addition of the 1.2 Kb cHS4 in the 3′LTR to the BG vector, termed BG-I resulted in reduction in titers by nearly 6-fold to 3.8±0.8×10⁷ IU/mL. A further addition of a 1.2 Kb PGK-MGMT internal cassette to the BG-I vector, termed BGM-I, did not reduce the titers any further (FIG. 20B). These data indicate that cHS4 insertion into the LTR, and not overall viral genome size reduced viral titers. Ramezani et al observed a 3-fold reduction in lentivirus titers when the 1.2 Kb cHS4 was inserted in lentivirus vectors encoding relatively small transgene expression cassettes (2 Kb in size or less). The present data is consistent with their results, although indicating a 6-10 fold reduction in titers with the addition of cHS4. It was additionally observed in the present study that reduction in titers by insertion of insulator elements in the LTR occurred by a distinct mechanism that was not dependent on the increased size of the viral genome.

### Example 37

### The Size of the Insert in the 3′LTR is Responsible for Reduction in Titers

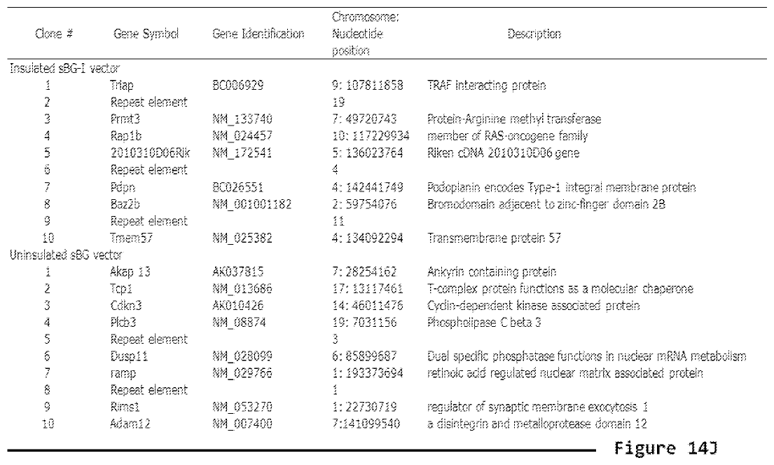

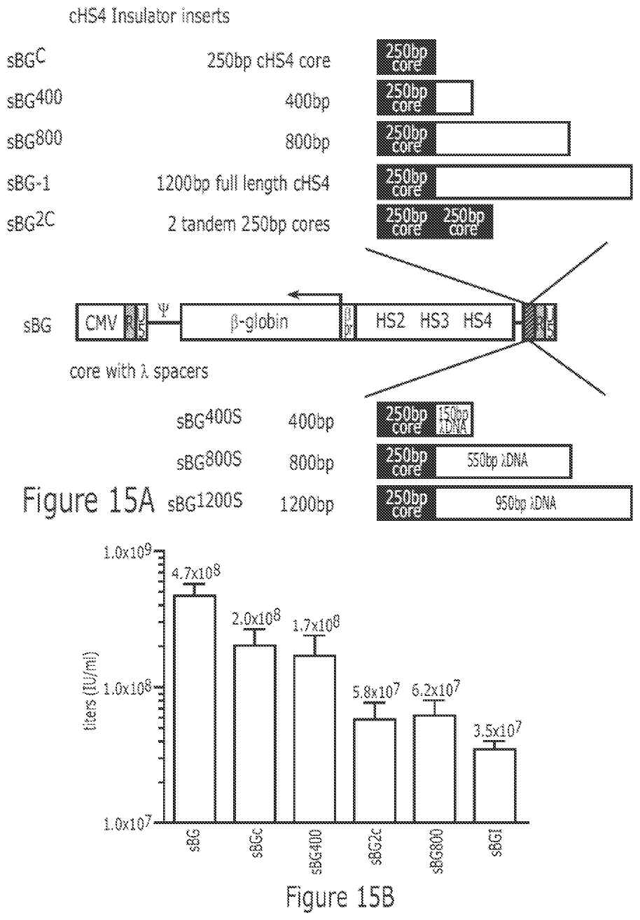

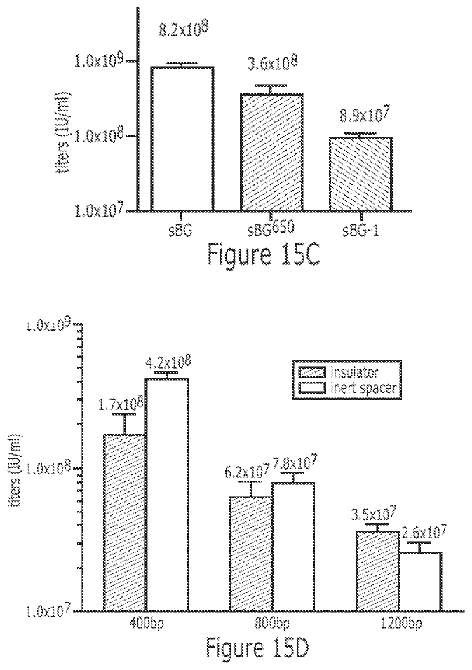

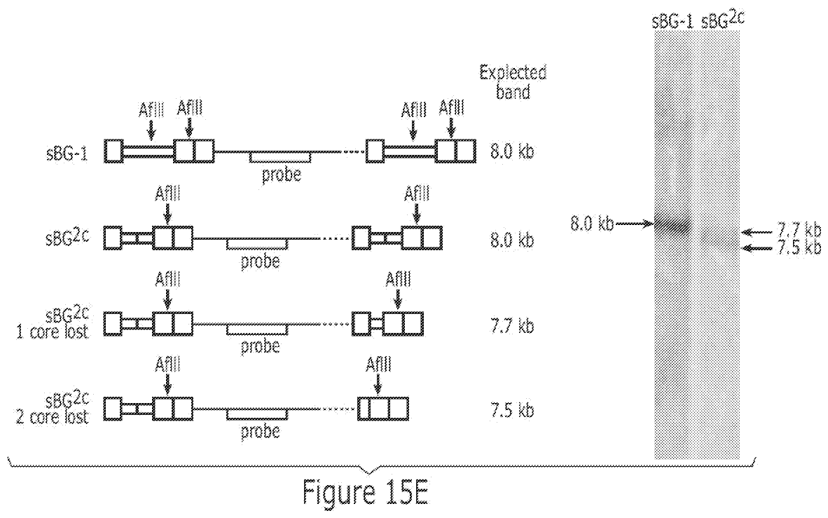

Although the LV vectors used did not exceed the natural size of the HIV-1 virus, the size of the cHS4 insert (1.2 Kb) exceeded the natural size of the wild type LTR (note that the wt LTR carries an additional 400 bp U3 enhancer, which is deleted from the self-inactivating 3′LTR). Experimentation was conducted to determine whether lowering of viral titers was due to lengthening of the SIN LTR beyond its natural capacity (400 bp), or whether titers were lower due to specific sequences in the insulator, which may potentially affect viral-RNA folding/binding to cellular proteins and thus limit packaging. A series of p-globin vectors were constructed in a self-inactivating lentivirus backbone, sSIN, carrying different length fragments of cHS4 in the 3′LTR (FIG. 15a): the first 250 bp of the insulator, also called the core, a 400 bp cHS4 fragment, matching the size of the U3 promoter/enhancer deletion in the 3′ SIN LTR, and a 800 bp cHS4 fragment, to generate sBG^{c}, sBG⁴⁰⁰, sBG⁸⁰⁰ vectors, respectively. These vectors were compared to an analogous ‘uninsulated’ vector, sBG, and a vector carrying the full-length 1.2 Kb insulator, sBG-I. In addition, a vector was cloned with two copies of the core as tandem repeats (250bpx2), sBG^{2C}. The cHS4 core has been shown to have 50% of enhancer blocking activity of the full length (1.2 Kb) insulator; the effect of the core has been shown to be copy number-dependent, with tandem repeats of cHS4 core reported to have the same insulating capacity as the full length 1.2 Kb cHS4.

Virus was generated from sBG, sBG^{C}, sBG⁴⁰⁰, sBG^{2C}, sBG⁸⁰⁰, sBG-I plasmids by concurrent transient transfections and concentration, and titered by flow cytometry of mouse erythroleukemia (MEL) cells infected with serial dilutions of the viruses, as described. MEL cells support adult type globin production. Each experiment was replicated four times.

It was determined that as the size of the cHS4 insert in the 3′LTR increased, viral titers dropped (FIG. 15b). There was a slight, but statistically significant reduction in titers with inserts of 250 bp and 400 bp. However, titers fell sharply thereafter, proportional to the length of the insulator fragment (FIG. 15b). The titers of the vector with a 1.2 Kb full-length cHS4 insulator, sBG-I were an order of magnitude lower than the uninsulated control vector, sBG. Of note, sBG^{2C} vector, with a tandem repeat of two cHS4 core sequences (500 bp insert) had titers similar to sBG⁸⁰⁰.

To ensure that reduction in titers was not from specific cHS4 sequences but an effect of the size of the LTR insert, three additional vectors were constructed, sBG^{400-s} sBG^{800-s} and sBG^{1200-s}. These vectors were analogous to sBG⁴⁰⁰, sBG⁸⁰⁰ and sBG-I, except that they contained spacer elements from the λ phage DNA downstream of the cHS4 core to generate 3′ LTR inserts of 400 bp, 800 bp and 1.2 Kb, respectively (FIG. 15a). The core cHS4 sequences were retained as the reduction in titers was minimal (and not observed in initial experiments) with the core; and it was important to determine if additional sequences downstream of the core are necessary for optimal insulator activity. The titers of the vectors containing DNA spacers were identical to those containing similar sized cHS4 fragments, and decreased with increasing size of the fragment in the 3′LTR (FIG. 15d). These data show that lengthening of the 3′ LTR lowered titers and this effect was not from specific sequences in cHS4. It has been reported that HIV-1 RT is not a strongly processive polymerase; it dissociates from its template frequently and the viral DNA is synthesized in relatively short segments. Therefore, it is likely that as the size of insert in the U3 LTR increased, there was reduced processivity through the 3′ LTR.

### Example 38

Recombination Occur with Repeat Elements in the 3 ′LTR

In order to detect if recombination events occurred in the LTRs from insertion of 2 copies of the core or different size fragments in the LTR, ˜12-20 MEL cell clones transduced with the entire series of insulated vectors (sBG^{C}, sBG⁴⁰⁰ sBG^{2C}, sBG⁸⁰⁰ and sBG-I) were generated. All clones that had a single copy of integrated provirus were identified using qPCR, as previously described. The 250 bp core from the genomic DNA of each clone was then amplified, by a standard PCR. The insulator core sequences could be amplified from clones derived from all vectors except those derived from sBG^{2C} transduced cells. In sBG^{2C} MEL clones, the insulator core was undetectable in 6 of 24 (25%) single copy clones by PCR, suggesting deletion of both tandem repeats of cHS4 core sequences in the 5’ and 3′ LTR of the provirus (FIG. 20D). To further analyze the frequency of recombined proviruses, a genomic Southern blot analysis on sBG^{2C} transduced MEL cell pools was performed. Genomic DNA from sBG^{2C} and sBG-I MEL cell populations was restricted with an enzyme that cut within the LTRs. FIG. 15E shows the expected lengths of the provirus with the sBG^{2C} vector and the sBG-I vector, used as a control. While a single proviral band was seen in sBG-I transduced MEL cells, the sBG^{2C} provirus in MEL cells showed loss of one or both copies of the cHS4 core sequences. Indeed, proviral bands containing two intact copies of the core were not detected at the level of sensitivity of Southern blot analysis. These data show that tandem repeats in sBG^{2C} recombined at a high frequency. The sBG2c vector, therefore, had lower viral titers from recombination events during reverse transcription, rather than the size of the LTR insert. These results were not unexpected, since repeat elements within gamma-retrovirus and lentivirus vectors have been shown to recombine frequently.

### Example 39

Steps in Vector Life-Cycle Affected by Large Inserts into the 3 ′LTR

Large viral genomes in RNA vectors have been shown to be limited at the level of RNA packaging. In the present study, there was no effect on titers with increasing the virus payload by 1.2 Kb, but titers decreased with increasing length of the insert in the LTR. Next, the mechanism by which this affected viral titers was explored. The following steps in the viral life cycle were studied: 1) characteristics of viral-RNA produced in packaging cells, 2) virus particle production, 3) post-entry steps: reverse transcription, nuclear translocation, integration and proviral integrity. For all of these studies, the vector with the largest insert, sBG-I was compared to the vector without the insulator, sBG.

### Example 40

Insertion of cHS4 in the 3′LTR does not Alter the Quantity or Quality of Viral-RNA in Packaging Cells

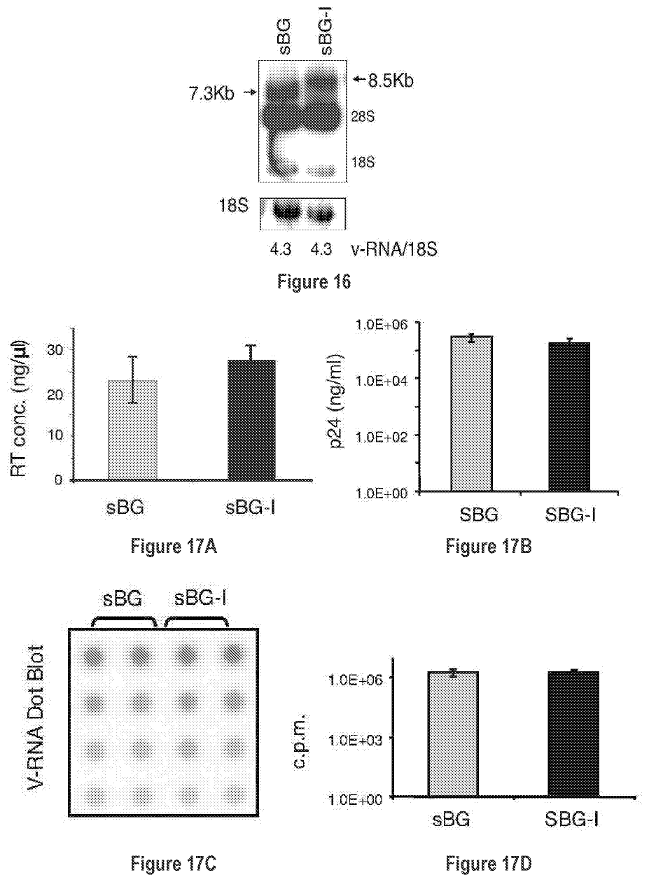

Northern blot analysis was performed on RNA derived from the 293T packaging cells after transient transfection with sBG, sBG-I vector plasmids, along with packaging plasmids (D8.9 and VSV-G). The blot was probed with hp fragment. FIG. 16 shows similar intensity viral-RNA transcripts of the expected lengths of sBG and sBG-I vectors. The probe non-specifically probed the 28S and 18S RNA. Nevertheless, there were no additional bands other than the full length-viral RNA of expected length, suggesting that no recombination or aberrant splicing occurred with insertion of the insulator. Thus, viral-RNA was produced efficiently in packaging cells, independent of the presence of an insert in the LTR.

### Example 41

Insertion of cHS4 in the 3′LTR does not Increase Read-Through Transcription

Experimentation was conducted to determine if the cHS4 insert upstream of the viral polyadenylation signal in the LTR could impair transcript termination of the viral RNA. Read-through transcripts have been shown to be excluded from encapsidation, and can lower viral titers. Although the northern blot in FIG. 16 showed the expected size viral-RNA band and no extraneous transcripts, it has been shown that transcriptional read-through is much less in lentivirus vectors, as compared to gamma-retrovirus vectors, that may not be readily detectable via a northern blot. Therefore a sensitive enzyme based assay was used to study read-through transcription.

Plasmid constructs were cloned, in which the wild type HIV-1 LTR, the SIN HIV-1 3′LTR with or without the insulator (from sBG-I or sBG vectors, respectively) were placed downstream of EF1-α promoter. A promoter-less IRES-cre cassette was placed downstream of the LTRs, so that cre expression would occur only from transcriptional read-through from the LTR. An EF1α-IRES-cre plasmid served as a positive control. Equal amounts of these plasmids were transfected into the reporter cell line, TE26, which expresses β-galactosidase proportional to cre expression. A GFP plasmid was co-transfected with the read-through plasmid constructs to normalize β-galactosidase activity for transfection efficiency. A plasmid carrying the truncated rat nerve growth factor receptor served as a negative control. A standard curve was generated that showed a linear correlation of the amount of the positive control IRES-cre plasmid transfected into cells and the β-galactosidase activity measured by spectrophotometer. No significant increase was observed in β-galactosidase activity from transfected constructs containing the insulated SIN lentivirus LTR, as compared to those carrying the SIN LTR without the cHS4 insulator. The results from the β-galactosidase assay were identical when confirmed by Lac-Z staining of TE26 cells plated on cover slips. These results showed that the insertion of cHS4 element upstream of the viral polyadenylation signal did not increase read-through transcription from the LTR.

### Example 42

Production of Viral Particles Containing Viral Genomes is not Affected by cHS4

To determine whether viral-RNA was encapsidated effectively into virions, p24 levels, virus associated reverse transcriptase (RT) activity and viral-RNA levels (FIGS. 17a-c) were measured. Virus was generated in an identical manner concurrently with the two vectors, and concentrated similarly in three separate experiments. To ensure purity of the viral preparation and lack of protein or plasmid contamination, virus was pelleted on a sucrose cushion and subjected to DNAse digestion for these experiments. Lack of plasmid contamination was confirmed by a qPCR for the ampicillin resistance gene, present in the plasmid backbone. The same volumes of virus preparation were then subjected to p24 ELISA and virus-associated RT assays; and viral-RNA was extracted for a dot-blot analysis. FIG. 17a shows that there was no difference in the amount virus-associated RT between the two vectors. The p24 levels in the sBG and sBG-I virus preparations were also similar (FIG. 17b). In order to ensure sBG-I virions contained viral genomes, and were not empty viral like particles; virus was subjected to RNA dot-blot analysis. FIGS. 17c-d shows one of two representative experiments. Viral RNA from sBG and sBG-I was loaded in duplicate in 4 different dilutions of p24 (FIG. 17c); and the intensity of the dots quantified by phosphoimager (FIG. 17d). There were similar amount of viral mRNA encapsidated from either vector. These data suggest that insertion of a 1.2 Kb fragment in the LTR did not affect packaging efficiency of viral mRNA or production of viral particles.

The present results with large inserts into the LTR are in contrast to those by Sutton and colleagues where lentivirus vectors with lengthened internal transgene cassettes are inefficiently packaged into virions. Equal amounts of virus particles produced from the sBG and sBG-I vectors, but significantly lower infectious/transduction titers suggests a post-entry block of large LTR insert bearing viruses, resulting in less integrated units.

### Example 43

Large LTR Inserts Affect Reverse Transcription and Integration of Viral cDNA

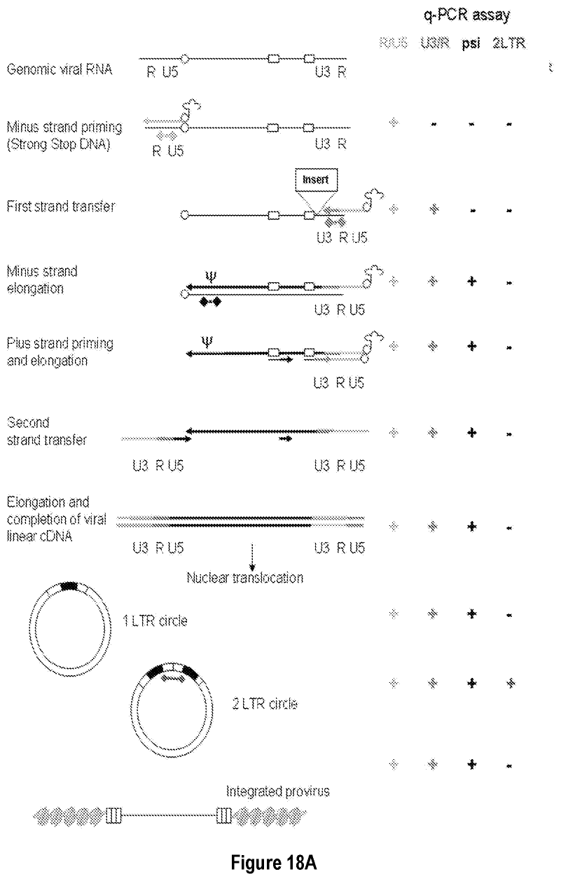

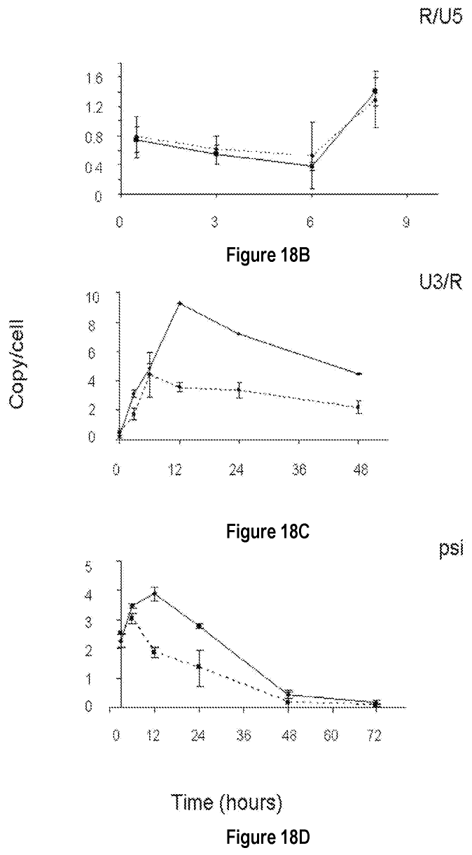

Post-entry steps were investigated; including reverse transcription, nuclear translocation, integration and proviral integrity. Reverse Transcription: the steps of reverse transcription, location of qPCR primers and probes and the viral DNA products are summarized in FIG. 18a. Reverse transcription initiates from the primer binding site near the 5′ end of the genomic RNA, and minus strand synthesis proceeds to the 5′ end of the genome (minus strand strong stop DNA (-sssDNA)). The newly formed -sssDNA anneals to the 3′R region of the genome (first strand transfer), minus-strand DNA synthesis resumes, accompanied by RNase H digestion of the viral RNA template. It has been shown that the secondary structure of viral RNA at the 3′ end is a critical determinant for the -sssDNA transfer, for the reverse transcription process to be efficient. Therefore, it is likely that presence of the insulator/an insert in the U3 region of the 3′ LTR would alter the secondary structure of the region involved in this complex process, resulting in overall decreased reverse transcription efficiency.

To assess reverse transcription efficiency, MEL cells were infected with equal amounts of sBG and sBG-I viral particles, based upon p24 levels, and cells collected at different time points post infection. Absence of plasmid contamination was confirmed by a qPCR for the ampicillin resistance gene present in the plasmid backbone (data not shown). Kinetics of early reverse transcription (production of -sssDNA) were studied using primers and probe spanning the R/U5 region (FIG. 18b). As expected, there was no difference detected in the kinetics between the two viruses, since the 5′ ends of sBG or sBG-I viral RNA were identical. Nevertheless, the data validated that qPCR accurately determined viral reverse transcription.

It is conceivable, however, that when RT switches templates (minus strand jump) to reverse transcribe the 3′ LTR, alteration of secondary structure from the presence of an insert in the U3 region would reduce reverse transcription products. Quantitative PCRs amplifying the U3/R and ψ regions were performed to quantify the amount of intermediate and late reverse transcribed viral cDNA in cells infected with sBG and sBG-I vectors, respectively (FIGS. 18c-d). It was discovered that RT efficiency soon after the first strand transfer was impaired. Notably, the U3/R primers amplified viral DNA that was reverse transcribed before the insulator sequences, suggesting that insert in the 3′LTR affected reverse transcription by altering or “poisoning” the 3′LTR. Indeed, the inefficiency in intermediate RT product formation was similar to that seen with late RT products. In both analysis, the peak of viral cDNA synthesis occurred at 12 h for the uninsulated vector sBG and then gradually decreased, consistent with integration of viral cDNA, and previously reported kinetics of reverse transcription. The amount of viral DNA from the insulated vector sBG-I was lower post-entry compared to sBG by about 2-fold at all time points, as early as 6 hours post-target cell entry. These data strongly suggest that reverse transcription after the minus strand jump was rate-limiting in the sBG-I vector.

Nuclear translocation: After the viral DNA is synthesized in the cytoplasm, it is translocated into the nucleus of infected cells, where it can be found as linear DNA or circular DNA (1-LTR and 2-LTR circles) (FIG. 18a). The linear form is circularized at the LTRs and is the direct precursor of the integration process; 1-LTR and 2-LTR circles, instead, are abortive products of homologous recombination and non-homologous DNA end joining, respectively. However, 1LTR and 2LTR circles are specifically localized in the nucleus, and are used as a marker for nuclear translocation. Presence of an insert in the LTR of lentiviruses can possibly interfere with the pre-integration complex (PIC) formation and the nuclear translocation of the viral DNA can lower transduction titers. It has been shown indeed that PIC complexes bind HIV LTR in the cytoplasm, and they are responsible for the transport to the nucleus and the integration of the cDNA into the genome of infected cells.

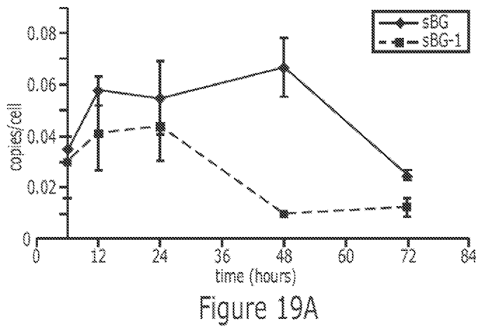

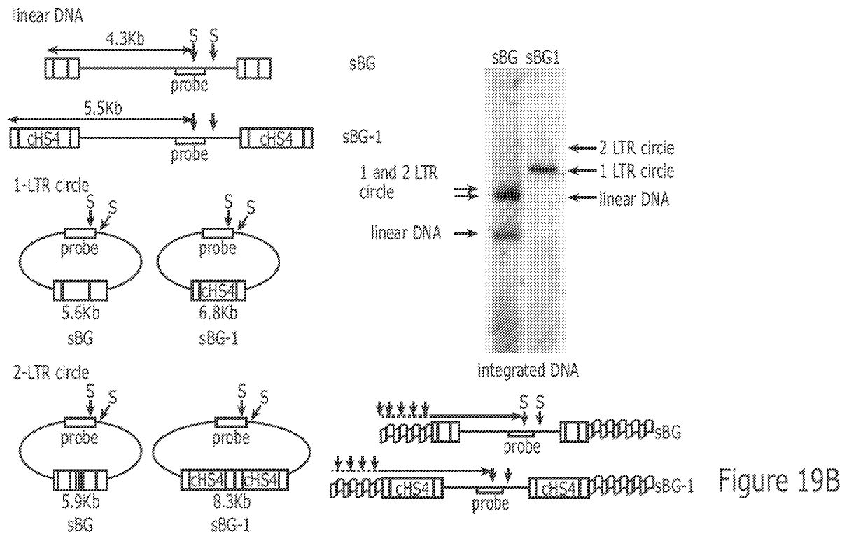

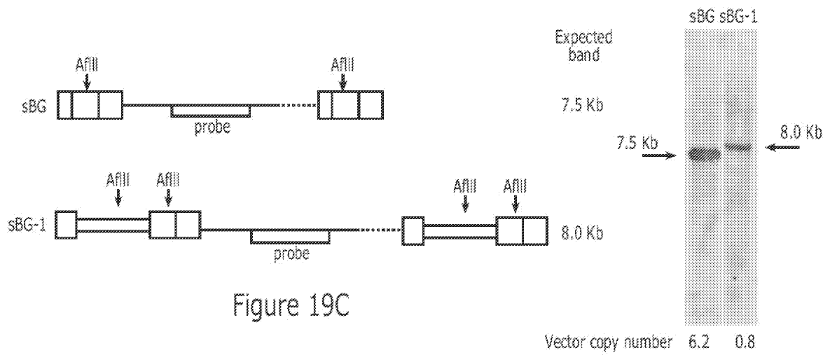

In order to detect the nuclear translocation, the amount of 2-LTR circles in both vectors were analyzed using a qPCR on DNA from infected MEL cells at different time points in sBG versus sBG-I infected cells. As shown in FIG. 19a, the amounts of 2-LTR circles were not significantly different between the two vectors at early time points. However, at 48 h after infection, the peak at which 2-LTR circles are normally detected, 2-LTR circles were 6.7 times higher in sBG infected cells, but were barely at the detection limit in sBG-I infected cells. Later time points (72 and 96 hours) were also analyzed, but no delay was determined in the kinetics of 2LTR circle formation in the insulated vectors. Indeed, the 2-LTR circles were barely detectable by qPCR in the sBG-I infected cells after 24 hours. These data suggested that nuclear translocation was likely reduced due to presence of the large U3 insert.

Integration: It is also conceivable, however, that two copies of large U3 inserts provide a template for homologous recombination, and the rate of homologous recombination between the two LTRs prior to integration increases, resulting in more 1-LTR circles and reduced 2-LTR circles (as proposed in the cartoon in FIG. 20). This would decrease the amount of template available for integration. Due to the nature of reverse transcribed viral cDNA with an insulated and uninsulated vector, 1LTR circles cannot be quantified by a PCR-based technique. Therefore, a Southern blot analysis was performed to detect linear viral cDNA, 1-LTR and 2-LTR circles at 72 hours post infection with equal amounts of sBG and sBG-I (quantified using p24 levels) (FIG. 19b). The Southern blot analysis showed that (i) the linear form of reverse transcribed viral cDNA, the form that integrates, was undetectable in the sBG-I lane at the sensitivity of Southern blot analysis, while it was readily detectable in the sBG lane. (ii) The 2-LTR circles were also undetectable in the Southern analysis in the sBG-I lane, but detectable in the sBG lane, corroborating the qPCR data on 2-LTR circles. (iii) However, large amount of 1-LTR circles were present in sBG-I lane, similar in amount to those seen in the sBG lane. The relative ratios of linear, 1- and 2-LTR circles in sBG versus sBG-I lanes suggested that there was increased homologous recombination of the sBG-I viral DNA. Indeed, these data indicated that nuclear translocation was not affected to any major extent by the U3 insert. But after the reverse transcribed cDNA entered the nucleus, increased 1-LTR circles, representing abortive recombinant integration products were formed due to the large LTR insert and therefore, integration was reduced.

It is conceivable that the integration machinery is also directly affected by the presence of foreign sequences in the LTR. Therefore, sBG and sBG-I viruses were packaged using an integrase defective packaging plasmid, so that effect of the insulator on reverse transcription, nuclear localization, and 1LTR circle formation could be studied independent of integration. The same analysis was performed as with active integrase containing viruses: a q-PCR to study the late reverse transcription product (using psi primers), 2LTR circles and a genomic Southern blot analysis to determine 1LTR circles and other forms of viral cDNA. The results were identical to those seen with sBG and sBG-I packaged with active integrase (shown in FIG. 19b): the same reduction was observed in late RT products and 2LTR circles by qPCR, but increased 1LTR circles by genomic Southern analysis (data not shown). Therefore, sequences inserted into the lentivirus LTR interfered mainly with the reverse transcription process, and increased the frequency of homologous recombination by a mechanism independent of the integrase machinery.

Finally, the integrated sBG and sBG-I provirus were analyzed for stability of transmission and efficiency of integration. The Southern blot analysis in FIG. 19b shows the integrated DNA as a smear, that is of higher intensity in the sBG than the sBG-I lane. In order to confirm and quantify integration, MEL cells were transduced with same amount of p24 levels of sBG or sBG-I virus, cultured for 21 days and a qPCR and Southern blot analysis were performed to compare proviral integration efficiency and stability (FIG. 19c). There were 6.2 proviral copies per cell in sBG MEL cell population by qPCR, while only 0.8 proviral copies were detected in sBG-I MEL cells, a 7.8-fold difference which is consistent with differences seen in transduction titers between the two vectors. Next, DNA was restricted with Afl-II, an enzyme that cuts within the LTRs (FIG. 19c, left panel). Consistent with transduction titers and qPCR, the amount of integrated sBG-I provirus was 8-fold less than sBG, as indicated by phosphoimager quantification of the Southern blot bands (FIG. 19c). The sBG-I vector did not recombine, as shown by the single proviral band of the expected size. Next, the full length insulator was detected by PCR in all single copy clones of sBG-I transduced MEL cells (FIG. 20D). Therefore, the linear sBG-I cDNA, albeit inefficiently formed, integrated as an intact provirus.

The overall reduced viral integration was primarily from a combination of inefficient reverse transcription and increased homologous recombination that hinder the availability of proviral DNA for integration. Since insulators are important for generating viral vectors that would be safe and provide consistent predictable expression, it is important to find a solution to the problem of low viral titers with insulated viruses. One way to overcome the problem would be to flank the internal expression cassette with cHS4 on either end, since further lengthening of the internal cassette did not decrease titers. However, this approach was not tried because repeat elements within retroviruses are known to result in recombination. Since HIV RT is known to have low processivity and frequently dissociate from its template, an attempt was made to increase the amount of RT delivered per vector particle, to assess if that would improve reverse transcription from large LTR inserts. RT was co-packaged in the virions as vpr-RT fusion protein. No significant increase in titers was observed when providing more RT in the virion. The next step was an attempt to increase the integrase (IN) per virion using the same strategy, and copackaged RT-IN-vpr fusion protein in the virion. There was a slight increase in titers providing RT-IN in the viral particle, but the difference was not significant.

Next, a detailed structure-function analysis of the 1.2 Kb cHS4 insulator was performed and a defined 650 bp sequences were determined as the minimum necessary sequences for full insulation effect. The titers of sBG650 were 3.6×10⁸ IU/mL, compared to a titer of 8.2×10⁸ IU/mL and 9.8×10⁷ IU/mL of the sBG and sBG-I vectors (FIG. 20C). Vectors with the 650 bp insert had very reasonable viral titers (2.2-fold lower titers than the uninsulated vector sBG, as compared to 9-10-fold lower titers of sBG-I) with no loss of insulator activity.

Ultimately it was determined that low transduction titers were not from an increase in size of the provirus, but increased length of the 3′LTR. The quantity and quality of viral RNA genomes produced were unaffected and viral-RNA encapsidation/packaging was comparable in vectors with and without a 1.2 Kb LTR insert. Reduced viral titers occurred from post-entry steps, from inefficient reverse transcription, increased homologous recombination in the LTRs of viral DNA, making less viral DNA available for integration. Improvements in vector design were made by including smaller insulator inserts that contained essential elements necessary for optimal insulator activity.

The present studies have important implications for future design of vectors with inserts within the 3′LTR, given the usefulness of chromatin insulator elements, customized lineage specific LTR vectors or double copy vectors.

### Example 44

### Vector Constructs

The cloning of the BG, BGM, BG-I and BGM-I vectors has been previously described. All other vectors were cloned into the sSIN backbone (details provided in Urbinati F, Xia P and Malik P, manuscript in review). All the vectors were obtained cloning the different insulator fragments into a unique Nhe I/EcoR V site was inserted in the U3 3′LTR region of the sSIN LV vector plasmid, which carried the human beta-globin gene and the hypersensitive site 2, 3 and 4 fragments, as previously described. Insulator fragments were amplified by PCR using the insulator plasmid pJCI3-1 as a template. All amplicons were sequenced following the PCR, and after insertion into the 3′LTR. The cloning of the uninsulated beta-globin vector and one that carrying the full length 1.2 Kb cHS4 insulator has been described previously. Briefly, the 1.2 Kb insulator fragment was obtained by digesting pJCI3-1 plasmid with Xba I and cloned into the Nhe I/EcoR V restriction site of sBG. sBGC was cloned inserting into sBG vector the fragment EcoR I/Xba I containing the 250 bp core from the pBS 1 core plasmid. The latter was obtained cloning the 250 bp core Insulator PCR product (using Core IF and Core 1R primers, as described herein) into BamH I/EcoR I restriction sites of a pBS plasmid. A second copy of the 250 bp core was then added into the pBS 1 core plasmid, cloning into EcoR I/Kpn I sites the PCR product (Core 2F and Core 2R), obtaining the pBS 2 core plasmid. 2 tandem copies of the 250 bp core were then isolated digesting the latter plasmid with Kpn I/Xba I, and then cloned into the sBG vector, obtaining sBG^{2C}. The sBG⁴⁰⁰ and sBG⁸⁰⁰ vectors were obtained cloning the 2 PCR products (using InsF and Ins400R primers and InsF and Ins800R primers, respectively) into the sBG Nhe I/EcoR V sites. sBG650 vector was obtained cloning the 3′ 400 fragment of the insulator in EcoRV/BspEI sites of sBG^{Ic} vector. The 3′ 400 fragment was PCR amplified from the plasmid pJCI3-1 using the following primers: 3′ 400 R (BspEI) and 3′ 400 F (EcoRV).

The vectors containing the A DNA spacers were obtained amplifying different size λ phage DNA using the following primer combinations: spacerF1 and spacerR1, spacerF1 and spacerR2 and spacerF1 and spacerR3 amplifying a 150 bp, 550 bp and 950 bp λ DNA fragments, respectively. The three PCR fragments were digested with Cla I and EcoR I restriction enzymes and ligated into EcoR I/Cla I sites in the pBS-1 core plasmid, The 400 bp, 800 bp and 1200 bp fragments were digested from the pBS-1 core plasmid with HincII and Xbal for the 400 bp fragment, and with Xba I and Xho I for the remaining two fragments, and cloned into the EcoR V/Nhe I restriction sites in the sBG vector. All the vectors cloned were confirmed by sequencing. The list of all the primers is available in (FIG. 20E).

### Example 45

### Cell Lines

Murine erythroleukemia cell (MEL) line and 293T cells were maintained in Dulbecco modified Eagle Medium (DMEM, Mediatech, Inc) supplemented with 10% heat inactivated fetal bovine serum (FBS) (U.S. Bio-technologies, Inc.). MEL cells were induced to differentiate in DMEM containing 20% FBS and 5 mM N, N′-hexamethylene bisacetamide (Sigma), as previously described. To derive single integrant clones, transduced MEL cells were cloned and clones were screened for β-globin sequences by PCR to identify transduced clones. Single copy clones were identified by qPCR for lentivirus γ-sequences, and a PCR for the cHS4 core sequences was performed on the single integrant clones to confirm presence of insulator sequences in the provirus.

### Example 46

### HbA Staining and FAGS Analysis

The staining using the anti-human HbA antibody was as previously described. Briefly, cells were fixed in 4% paraformaldehyde for 60 minutes at room temperature, washed once with phosphate-buffered saline (PBS), and the pellet resuspended in 100% methanol for 5 minutes. The fixed cells were then washed with PBS, and nonspecific antibody (Ab) binding was blocked using 5% nonfat dry milk for 10 minutes at room temperature. Subsequently, cells were washed in PBS, pelleted, and permeabilized. The cells were divided into 2 tubes and stained with either anti-Zeta globin-fluorescein isothiocyanate (FITC) (1 μg/10⁶ cells) as a negative control or anti-HbA-FITC (0.1 μg/10⁶ cells) (Perkin Elmer) for 30 minutes at room temperature in the dark. Unbound Ab was removed by a final wash with PBS before they were analyzed on FACS Calibur (Becton Dickinson).

### Example 47

### Virus Production

Virus was produced by transient cotransfection of 293T cells, as previously described, using the vector plasmids, the packaging (A8.9 or A8.2 for active or inactive integrase respectively) and the VSV-G envelope plasmids; virus-containing supernatant was collected at 60 hours after transfection and concentrated by ultracentrifugation. All vectors in an experiment were packaged simultaneously. Virus was treated with DNase and/or Dpnl to remove plasmid DNA contamination and layered on a 20% sucrose cushion to obtain purified viral particles for specific experiments on vector life cycle indicated in the results. Virus was concentrated 1400-fold from all viral supernatants after ultracentrifugation at 25,000 rpm for 90 minutes. Viral titers were determined by infecting mouse erythroleukemia (MEL) cells with serial dilutions of concentrated virus, differentiating them, and analyzing them for HbA expression by fluorescence-activated cell-sorter scanner (FACS).

### Example 48

### Northern Blot

Total RNA was extracted from 293T cells using RNA-STAT (Tel-Test, INC, Texas), 72 hours after transfection. Northern Blot was then performed according to standard protocol. The blot was hybridized with a ³²-P labeled β-globin probe.

### Example 49

### RNA Dot Blot

Viral-RNA was extracted from same volumes of concentrated viruses using the QIAamp Viral RNA Mini Kit (Qiagen, Valencia, CA) following the manufacturer's instructions. Briefly the virus was lysed under a highly denaturing condition and then bound to a silica-gel-based membrane. Two washing steps efficiently washed away contaminants and v-RNA was eluted in 30 μl of DEPC-H2O. After elution viral-RNA was treated for 20 min. at room temperature with amplification grade DNAse I (Invitrogen). DNase was inactivated incubating the sample at 65°. Viral RNA was then denatured in 3 volumes of denaturation buffer (65% formamide, 8% formaldehyde, MOPS 1×) for 15 min at 65°. After denaturation 2 volumes of ice-cold 20×SSC were added and the RNA was bound to a nylon membrane by aspiration through a dot-blot apparatus. The blot was hybridized with a ³²-P labeled β-globin specific probe and a film was exposed overnight. Quantification of the dots was performed with a phosphoimager (Biorad, Hercules, CA).

### Example 50

### Reverse Transcriptase Assay

Concentrated virus (1 μL), and serial dilutions (1:10, 1;100, 1:1000) were lysed and processed following the “Reverse transcriptase (RT) assay, colorimetric” Kit (Roche) protocol. Briefly concentrated viral particles were lysed with lysis buffer and viral-RNA reverse transcribed using digoxigenin and biotin-labeled nucleotides. The detection and quantification of synthesized DNA as a parameter of RT activity followed a sandwich ELISA protocol: biotin-labeled DNA was bound to the surface of microplate modules that were pre-coated with streptavidin. In the next step, an antibody to digoxigenin, conjugated to peroxidase (anti-DIG-POD), was bound to the digoxigenin-labeled DNA. In the final step, the peroxidase substrate ABTS was added, that resulted in a colored reaction product that was quantified using an ELISA reader at a wavelength of 405 nm. The amount of colored product directly correlated to the level of RT activity in the sample.

### Example 51

### P24 Assay

P24 antigen concentration was determined by HIV-1 p24 Antigen EIA Kit (Beckman Coulter). Briefly, serially diluted virus was lysed and incubated onto p24 antigen coated microwells, and washed following manufacturer's protocol. Color absorbance was measured using a spectrophotometer at a wavelength of 450 nm. p24 assay was performed in duplicate.

### Example 52

### Southern Blot

To analyze the integrity of the provirus we infected MEL cells, expanded them for 21 days and extracted DNA using Qiagen Blood and Cell culture DNA Mini Kit (Qiagen). 10 μg of DNA was digested with Afl II, an enzyme that cuts in the LTRs. To determine presence of viral linear DNA, genomic DNA was extracted 72 h after infection of MEL cells and restricted with Stu I, an enzyme that cuts twice within the provirus. The DNA was separated on a 0.8% agarose gel, transfer to a nylon membrane, and probed overnight with a β-globin fragment.

### Example 53

### Real Time PCR for RT Products and 2LTR Circle

The same amount of p24 was used to transduce MEL cells with sBG and sBG-I vectors, in DMEM media, in the presence of 8μ, g/mL polybrene. Cells were harvested at different time point (0.5 h, 3 h, 6 h, 8 h, 12 h, 24 h, 48 h, 72 h) and DNA extracted using Qiagen Blood and Cell culture DNA Mini Kit (Qiagen). Genomic DNA (50 ng) from a single copy MEL clone (confirmed by Southern for a single integrant) was diluted with untransduced DNA to generate copy number standards (1-0.016 copies/cell). The primers and the probe for RT product were designed using the Primer Express Software from Applied Biosystems, Foster City, CA. Primers and probe sequence for early RT products (R/U5) qPCR assay are: forward primer 5′-GAACCCACTGCTTAAGCCTCAA-3′ (SEQ ID NO: 10), reverse primer: 5′-ACAGACGGGCACACACTACTTG-3′ (SEQ ID NO: 11) The reaction was carried out with TaqMan MGB Probe: 5′-AAAGCTTGCCTTGAGTGC-3′ (SEQ ID NO: 12). Primers and probe sequence for intermediate RT products (U3/R) qPCR assay are: forward primer 5′-CCCAGGCTCAGATCTGGTCTAA-3′ (SEQ ID NO: 13), reverse primer: 5′-TGTGAAATTTGTGATGCTATTGCTT-3′ (SEQ ID NO: 14) The reaction was carried out with TaqMan MGB Probe: 5′-AGACCCAGTACAAGCAAAAAGCAGACCGG-3′ (SEQ ID NO: 15). For the late RT product assay (psi) the primers were designed to recognize the ψ region of the provirus: forward primer: 5′-ACCTGAAAGCGAAAGGCAAAC-3′ (SEQ ID NO: 16), reverse primer: 5′-AGAAGGAGAGAGATGGGTGCG-3′ (SEQ ID NO: 17). The reaction was carried out with TaqMan Probe: 5′-AGCTCTCTCGACGCAGGACTCGGC-3′ (SEQ ID NO: 18) with TAMRA dye as quencher. Normalization for loading was carried out using mouse apoB gene controls. The cycling conditions were 2 min at 50° C. and 10 min at 95° C., then 40 cycles of 95° C. for 15s and 60° C. for 1 min. The primers and probe for 2LTR circle were as previously described. The PCR mixture was thermocycled according to the thermal cycler protocol for 96 well plates in Applied Biosystems 7900HT Fast Real-Time PCR System Base Unit.

### Example 54

### LSDs and Treatments-Generally

Lysosomal storage disorders (LSD) include about 50 metabolic diseases that collectively affect approximately 1 in 5000 live births with—65% affecting the CNS. Mucopolysaccharidosis type I (MPS I, or Hurler Syndrome for its severe form), one of the most common LSD, is caused by defective IDUA and consequent systemic accumulation of the unprocessed glycosaminoglycans (GAG) (1). Treatment modalities for LSDs are currently limited to bone marrow transplantation (BMT) and enzyme replacement therapy (ERT). These approaches while providing significant promise for treatment of the visceral manifestations of LSDs, do little to address CNS pathologies for this group of disorders. Moreover, BMT is limited by procedure-related mortality between 20 and 30%, late complications such as graft versus host disease, and by the need to find an HLA-matched donor. Pharmaceutical lysosomal enzyme products are available for several LSDs and are being used to ameliorate visceral manifestations in some LSD patients. However, it is limited by poor penetration of the CNS, the need for frequent intravenous infusion for a lifetime and by tremendous costs. A new therapeutic approach to treatment of LSDs with lower mortality and morbidity, and with the capacity to correct CNS deterioration is needed.

### Example 55

### HSC and Erythroid Cell Gene Transfer-General

Ex vivo HSC gene transfer followed by autologous transplantation is an attractive alternative for LSD treatment that could provide life-long therapeutic effects without the morbidity and mortality of allogeneic transplantation. However, in general the frequencies of transduced and successfully engrafted HSC have been low in gene therapy clinical trials. In addition, inadvertent activation of cellular proto-oncogenes by ubiquitous LTR promoters resulted in secondary leukemogenesis in two otherwise successful clinical trials (11-13).

Healthy individuals can produce 2.4×10¹¹ RBC per day with a daily output of 7.2 g of hemoglobin. Redirecting a portion of the formidable protein synthesis machinery in maturing erythroid cells toward the expression of a transgene can provide an efficient approach for long-term protein delivery into the circulation. Moreover, the high efficiency of protein synthesis can compensate for the generally low HSC gene transfer frequency in gene therapy clinical trials. Restricting transgene expression to a subset of HSC offspring can also reduce the risk of insertional oncogenesis. To that end, an ankyrin-1 based erythroid specific hybrid promoter/enhancer (IHK) can introduce high erythroid-specific expression in vivo in primary and secondary murine BMT recipients (17).

### Example 56

### Reprogramming Erythroid Cells for Production of Alpha-L-Iduronidase (IDUA)

Restricting transgene expression to maturing erythroid cells can reduce the risk of activating oncogenes in hematopoietic stem cells (HSCs) and their progeny, yet take advantage of their robust protein-synthesis machinery for high-level protein production. This study indicates that an erythroid-specific hybrid promoter can provide inducible IDUA expression and release during in vitro erythroid differentiation in murine erythroleukemia cells, resulting in phenotypical cross-correction in an enzyme-deficient lymphoblastoid cell line derived from patients with Mucopolysaccharidosis (MPS) Type I. Stable and higher-than normal plasma IDUA levels were achieved in vivo in primary and secondary MPS I chimeras for at least 9 months after transplantation of HSCs transduced with the erythroid-specific IDUA-containing lentiviral vector (LV). Moreover, long-term metabolic correction was demonstrated by normalized urinary glycosaminoglycan accumulation in all treated MPS I mice. Complete normalization of tissue pathology was observed in heart, liver and spleen. Notably, neurological function and brain pathology were significantly improved in MPS I mice by erythroid-derived, higher-than-normal peripheral IDUA protein. These data are the first to demonstrate that late-stage erythroid cells, transduced with a tissue-specific LV, can deliver a lysosomal enzyme continuously at supra-physiological levels to the bloodstream, and can correct the disease phenotype in both viscera and CNS of MPS I mice. This approach provides a paradigm for the utilization of red blood cell precursors as a depot for efficient and potentially safer, systemic delivery of non-secreted proteins by ex vivo HSC gene transfer.

### Example 57

Inducible IDUA Expression and Enzyme Release from IHK Promoter During In Vitro Erythroid Differentiation in MEL Cells.

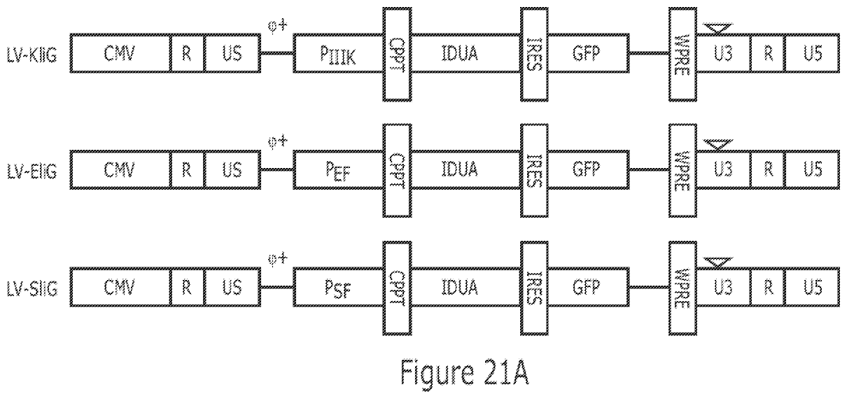

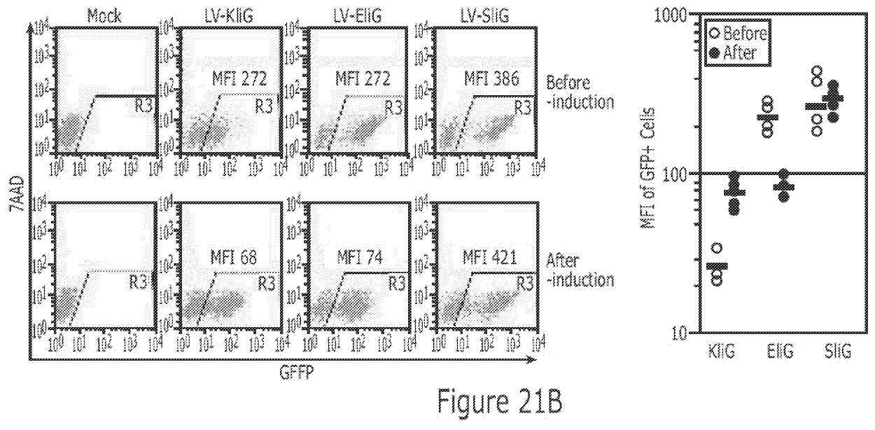

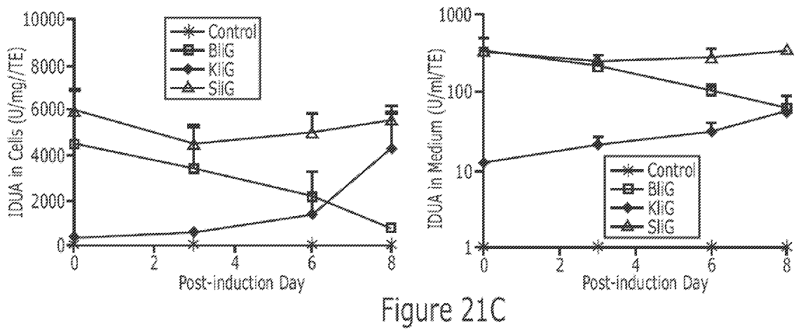

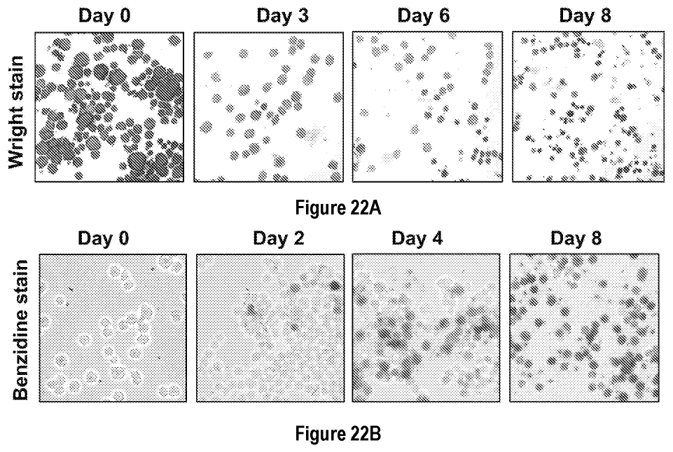

To determine if cells from the erythroid lineage could produce and release lysosomal IDUA during erythroid differentiation, an erythroid MEL cell line was used to compare IDUA expression and enzyme release from three LV constructs containing the same expression cassette with three different promoters, i.e., erythroid specific IHK, ubiquitous cellular promoter of human elongation factor-1a (EF) and LTR promoter of spleen focus-forming virus (SF) (FIG. 21A). Progressive erythroid differentiation during HMBA-induction of MEL cells was confirmed by morphologic evaluation, and by histochemical staining with Benzidine showing an increasing number of hemoglobin-expressing cells (FIG. 22). The mean fluorescent intensity (MFI) of GFP in stably transduced MEL-KIiG increased from mean of 27 to 78 by Day 8 of inductive culture; while the MFI in MEL-EliG decreased from 228 to 84 and no significant change of MFI was observed in MEL-SIiG during erythroid induction (FIG. 21B). IDUA expression from IHK was relatively low (5% of SF and 8% of EF) in un-induced MEL-KIiG, but increased 15-fold following induction, reaching an intracellular level similar to that obtained with the strong LTR promoter of SF (FIG. 21C). After erythroid induction, IDUA expression from the EF promoter decreased to 17% of the un-induced levels, while the levels from SF promoter remained unchanged. A similar pattern was found in IDUA activity in the media from transduced MEL cells during induction. The endogenous IDUA levels of un-transduced MEL control cells were very low (1.1±0.7 U/mg) and decreased to negligible levels during erythroid induction; no IDUA activity was ever found in culture medium. These results demonstrate that maturing erythroid cells can increasingly overexpress IDUA during differentiation to levels comparable to strong SF promoter, and a portion of the IDUA can be released from these cells.

### Example 58

Erythroid Released Enzyme Cross-Corrected Lysosomal Defect in Cells Derived from a MPS I Patient.

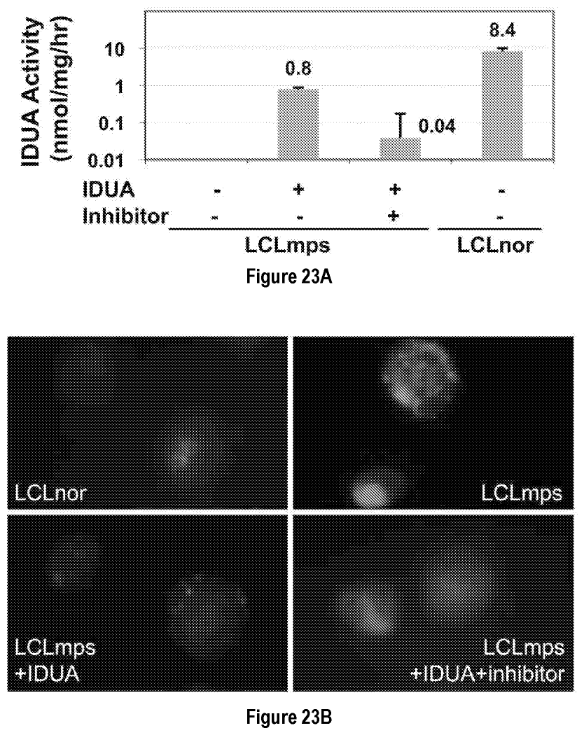

IDUA is synthesized in the endoplasmic reticulum as a 653-amino-acid precursor that undergoes post-translational glycosylation and extensive proteolytic processing to produce at least 10 polypeptides during passage through the endosome-lysosome compartments (18). The enzyme is normally targeted to the lysosome via the cation-independent mannose 6-phosphate (M6P) receptor (MPR) (19). To test if this endogenous uptake pathway remains effective for IDUA protein released by erythroid cells, lymphoblastoid cells derived from an MPS I patient were exposed to medium preconditioned by induced MEL-KIiG (FIG. 23). The intracellular IDUA levels increased from undetectable to 0.8 U/mg or about 10% of wild-type levels (FIG. 23A). This uptake process was inhibitable by the presence of M6P competitor.

The increased abundance of lysosomes and the abnormal lysosomal morphology in MPS I cells are direct consequence of GAG accumulation. To determine the functional integrity of IDUA generated by erythroid cells, in situ immunostaining was performed using a fluorescent dye that could be endocytosed into lysosomes (FIG. 23B). In contrast to normal LCL cells, untreated LCLmps cells contained more lysosomes, i.e., stronger fluorescent intensity, and these compartments might be smaller in size, as suggested by more uniform staining. The majority of LCLmps cells exposed to erythroid-released IDUA exhibited a normalized lysosomal pattern and this was not seen in the presence of M6P. These data demonstrate that IDUA released from maturing red cells can use the MPR lysosomal enzyme trafficking system, and also can restore a normal pattern of lysosomal distribution and morphology in cells derived from MPS I patients.

### Example 59

Long-Term, Supra Physiological Levels of IDUA were Achieved in Plasma of MPS I Mice Transplanted with LV-KIiG Transduced Enzyme-Deficient HSCs.

In vivo systemic IDUA production by erythroid-specific LV in MPS I mice (FIG. 24) was evaluated. HSC-enriched Lin bone marrow cells from MPS I mice were isolated by lineage depletion with 92-97% purity, followed by transduction twice with LV-KIiG or LV-EliG for a total MOI of 20 or 18, respectively. Starting 2 weeks after BMT, plasma IDUA activity levels increased from undetectable levels to 27±9 U/ml, and persisted at supra-physiological IDUA activity levels (4-fold higher than wild-type) till the end of the 5-month observation period (FIG. 24A). Only 0.7+0.2 U/ml of plasma IDUA was present in MPS I mice that received wild-type marrow, and 7±4 U/ml were found in those receiving LV-EliG transduced Lin cells. These results show that a lysosomal enzyme can be produced and released into the circulation in vivo by erythroid cells using tissue-specific LV, even though erythroid cells are not normally regarded as cells that secrete plasma proteins.

To determine whether gene transfer had occurred in primitive HSCs and could sustain long-term erythroid IDUA “secretion”, secondary transplantation in MPS I mice was conducted using bone marrow from primary recipients of LV-KIiG transduced cells 5 months after primary BMT (FIG. 24B). Stable IDUA erythroid expression derived from primary transduced HSCs was attained in all secondary recipients sampled 8 weeks and 16 weeks after transplantation. Long-term plasma IDUA levels achieved in the secondary MPS I recipients were about 8-fold higher than wild-type levels.

To determine transgene frequency in primary and secondary BMT recipients, GFP transgene frequency by real-time qPCR in peripheral blood leukocytes and total bone marrow 4-5 months after transplantation was evaluated (FIG. 24C). Similar levels of transgene frequency were obtained with both EliG and KIiG vectors in primary recipients, averaging 22±7% and 23±8% in PBL, and 24±12% and 28±16% in bone marrow, respectively. Stable gene transfer in HSCs was ascertained in secondary recipients for KIiG with 22±3% GFP⁺ in PBL and 24±9% in BM.

Five months after primary transplantation, spleen colony-forming unit assays were carried out to determine transgene frequency and functional IDUA expression in the clonal progeny of LV-KIiG transduced pluripotent hematopoietic stem/progenitor cells after secondary transplants (FIGS. 24, C and D). Of 112 CFU-S analyzed for the KIiG group of mice, 35 CFU-S were positive for the provirus determined by real-time qPCR (31%), and all expressed elevated IDUA determined by enzyme assay. Of these, 32 colonies contained a single copy and 3 colonies contained 2 copies of provirus. The IDUA activity levels in MPS I CFU-S harboring single-copy KIiG insertion were 100±59 U/mg, which were 9-fold higher than those derived from heterozygous mice (11±6). The mean IDUA levels in LV-KIiG transduced 2-copy CFU-Ss was 299±86 U/mg, i.e., 12.5-fold higher than those derived from wild-type mice (24±16). These observations were consistent with robust levels of IDUA detected in plasma of secondary MPS I recipients transplanted with primary LV-KIiG transduced HSCs.

To determine whether erythroid specific IDUA expression may affect normal erythropoiesis, complete blood count was performed in primary or secondary MPS I chimeras 5-6 months after transplantation. Erythrocyte parameters, such as hemoglobin levels, RBC counts, hematocrit values and red blood cell distribution width etc., were indistinguishable between MPS I chimeras receiving KIiG-transduced HSC and those receiving WT bone marrow (FIG. 25). These results indicate that no significant perturbation of erythropoiesis occurred from IDUA transgene expression in these animals.

### Example 60

### Erythroid-Specific Expression of IDUA Predominantly in Late Stages of Erythroblasts.

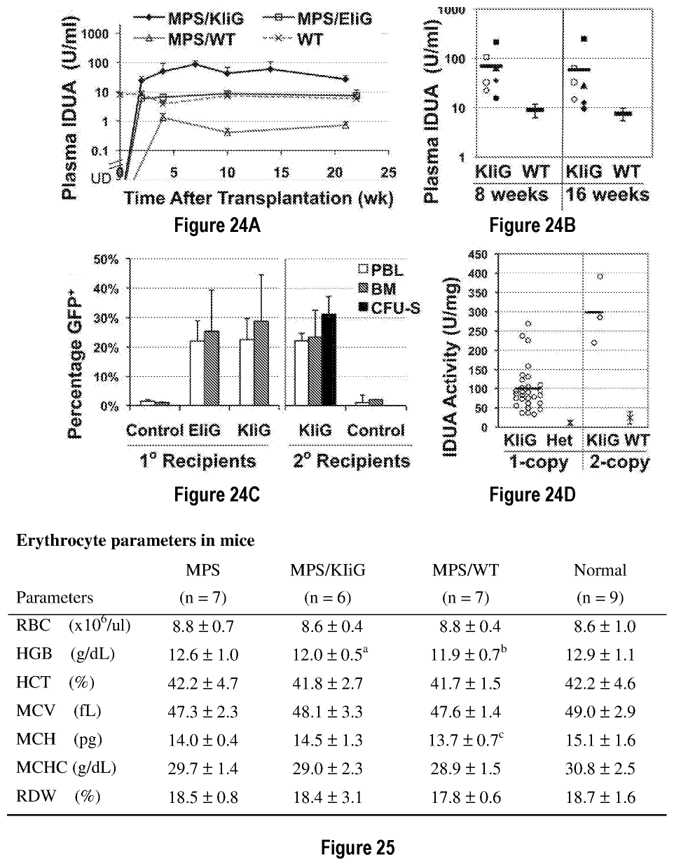

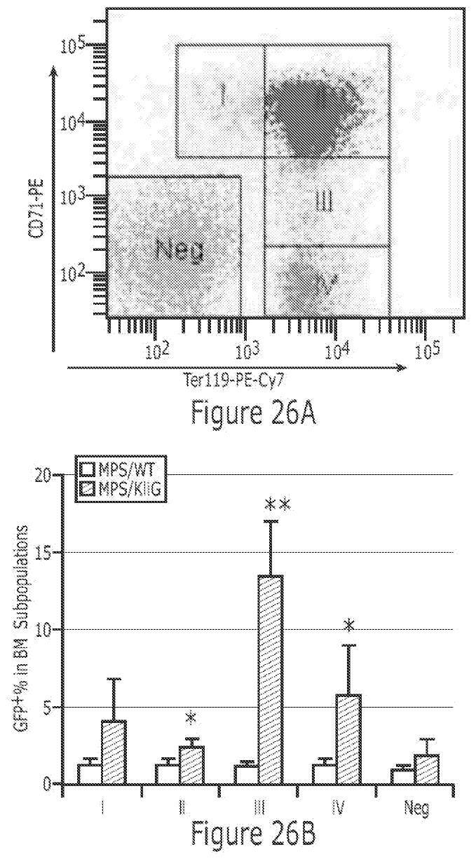

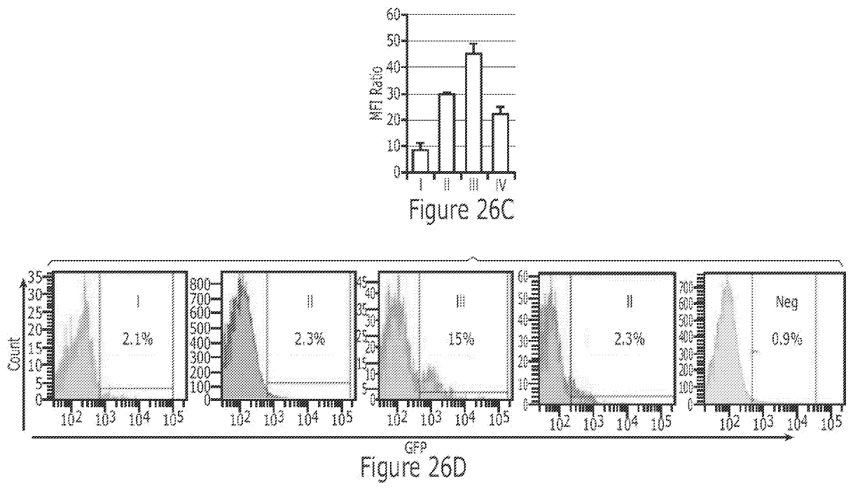

To investigate whether IDUA expression from LV-KIiG is erythroid specific and define its expression pattern during erythroid differentiation in vivo, GFP expression (as bicistronic gene downstream from IDUA) was evaluated in fresh bone marrow cells stained with the erythroid-specific cell surface makers Ter119 (glycophorin A-associated protein) and CD71 (transferrin receptor) (FIG. 26). The progressive maturation in erythroid precursor subpopulations, labeled as I-IV on the histograms, has been examined previously by us (20) and others (21, 22). Population I corresponded mainly to proerythroblasts and early basophilic erythroblasts. Population II contained a mixture of basophilic, polychromatophilic, and orthochromatic erythroblasts. Population III contained reticulocytes and a fraction of mature RBC, while Population IV was mostly RBC. GFP-expressing cells became detectable, i.e., significantly higher than background levels, starting from subpopulation II and further increased with greater percentage representation in later stages of erythroid differentiation (FIG. 26B). Only background levels of expression were observed in non-erythroid populations (Ter119-CD71-fraction). The GFP expression levels, as determined by MFI, increased in population II, peaked in population III, while it decreased again in population IV consisting of enucleated, mature red blood cells (FIG. 26C). These results demonstrate predominant transgene expression in late stages of erythroid differentiation, and confirm the erythroid restriction imparted by the IHK promoter as previously found with related β-globin-encoding vectors (17).

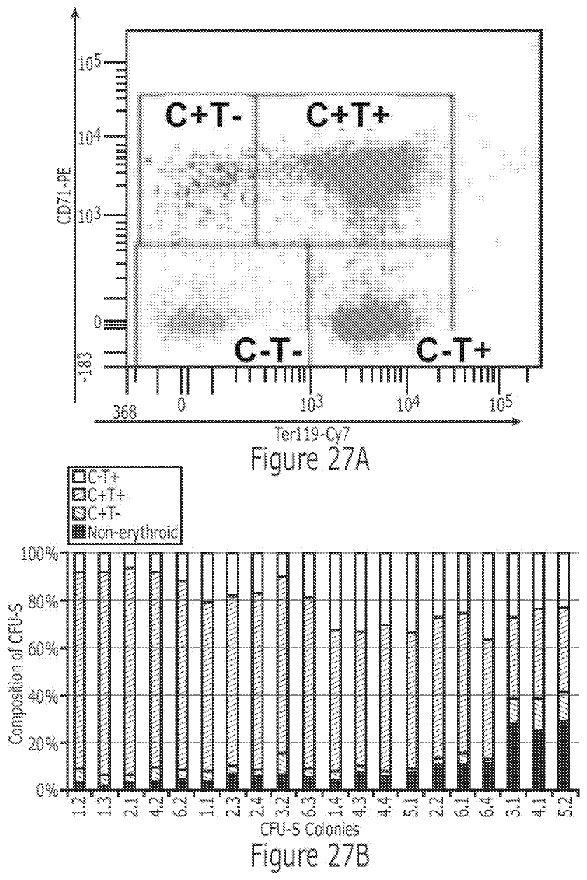

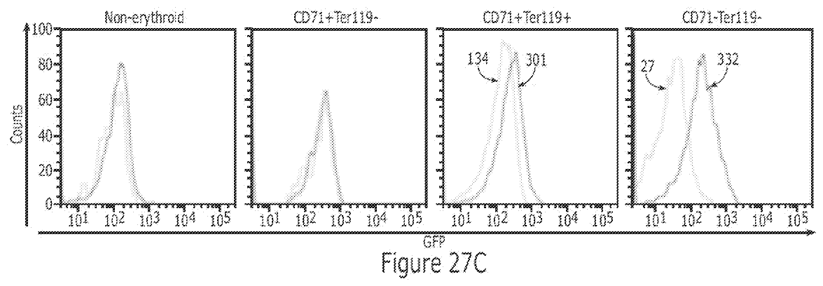

To evaluate transgene expression in the clonal progeny of HSC, the cellular composition of individual CFU-S, and GFP expression pattern in transduced colonies were examined (FIG. 27). Twenty CFU-S colonies were immunostained with erythroid markers CD71 and Ter119, and all of them contained 48-91% erythroblasts/reticulocytes (CD71+) (FIGS. 27A and 27B), suggesting that 12-day CFU-S colonies were either erythroid or multi-lineage colonies. Moreover, GFP expression was restricted in mid/late stages of erythroblasts and reticulocytes (Ter119+ subpopulations) (FIG. 27C).

### Example 61

### Long-Term Systemic Metabolic Correction in MPS I Mice.

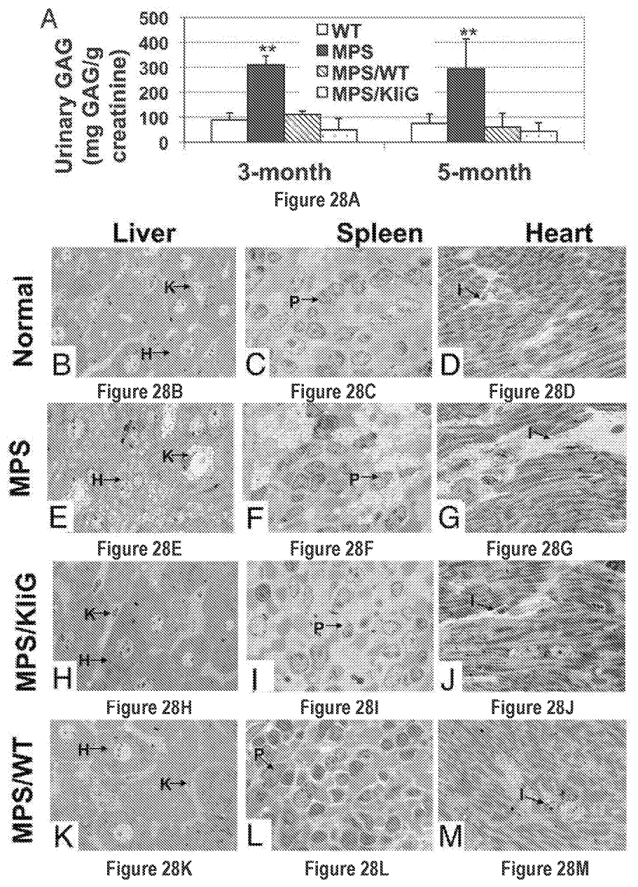

To evaluate the therapeutic effect of transduction, the GAG levels, a parameter for systemic metabolic accumulation, were determined in urine of treated MPS I groups, in comparison with age-matched untreated MPS I and normal mice (FIG. 28). At 3-months and 5-months after transplantation, the mean GAG levels for all transplanted groups were not different from those in normal controls. Untreated MPS I mice had significantly higher urinary GAG accumulation than all other groups (p<0.01). These data indicate that significant systemic metabolic correction of storage disease can be achieved in MPS I chimeras receiving MPS I HSCs transduced with an erythroid specific LV or receiving wild-type HSCs.

To evaluate the potential therapeutic effects of erythroid-derived IDUA on multi-organ deficits in MPS I mice, histological examinations of liver, spleen and heart were performed on two mice from the KIiG group (one with the highest plasma IDUA and the other with the lowest), two from MPS I mice transplanted with WT marrow, and compared with untreated MPS I and wild-type animals (FIG. 28B to M). Cytoplasmic vacuoles, which represent distended lysosomes from which the GAG contents have been leached by fixation, are the pathognomonic feature of Mucopolysaccharidoses. In age-matched untreated MPS I mice (7-8 months old), lysosomal inclusions were most marked in Kupffer cells that were distended by massive vacuoles, as well as in hepatocytes (FIG. 28E). Extensive pathological vacuoles were also observed in scattered perisinusoidal cells of the spleen (FIG. 28F). The interstitial space between myocardial cells was distended by aggregates of vacuolated interstitial cells in the heart of untreated MPS I mice (FIG. 28G). In contrast, tissues of all tested peripheral organs from MPS I mice transplanted with MPS HSC that were transduced by LV-KIiG, or those transplanted with WT HSC contained no vacuolated cells (see Material and Methods for details), and were indistinguishable in regions examined from those of age-matched wild-type controls. These results indicate that erythroid-generated recombinant IDUA enzyme can correct lysosomal storage pathology completely in the liver, spleen and heart.

### Example 62

Significant Improvement in Neurological Function and Brain Pathology in MPS I Mice with Higher-than-Normal IDUA Activities in Peripheral Blood.

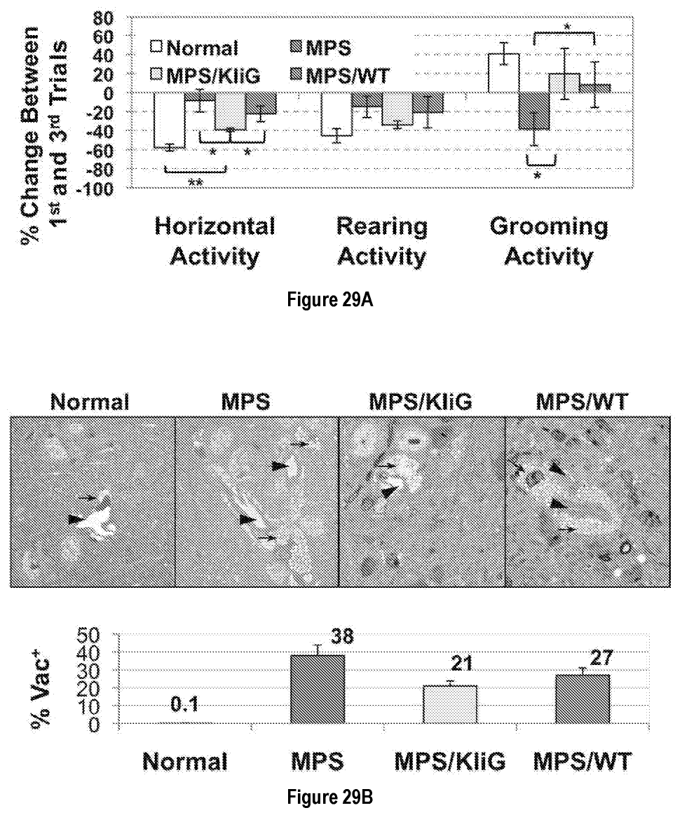

To determine if the supra-physiological levels of IDUA in the circulation could lead to functional neurological improvement in MPS I mice, a repeated open-field test was conducted (FIG. 29). This test has been shown previously to characterize nonaversive and non-associative memory deficits in MPS I mice without gender differences (23). Mice were exposed to the same open-field for 3 repeated trials with a 30 min inter-trial interval. The normal mice showed a 58% reduction in horizontal locomotor activity, whereas the MPS I mice only showed a 9% reduction in activity (p<0.001). Importantly, mice from the MPS/KIiG group exhibited an intermediate level of habituation (39% reduction in locomotor activity), a significant improvement toward normal behavior (p<0.05 compared with MPS I). Whereas MPS/WT group showed no significant improvement. In addition, the normal mice spent 41% more time grooming in the final trial than in the first; however, the untreated mice spent 39% less time in the final trial (p<0.001). Both treated groups showed significantly normalized grooming behavior with 20% more for MPS/KIiG mice and 4% more time grooming for MPS/WT group. Treated mice also had a greater reduction in rearing on the last trial compared to un-treated controls. These observations indicated a significant improvement of the memory deficit in MPS I mice by erythroid derived IDUA in peripheral blood.

The histological appearance of forebrain tissues were then compared (FIG. 29B). Cells that were distended with pathologic vacuoles were still visible in cerebral cortex of KIiG treated mice and MPS mice transplanted with WT marrow; however, there appeared to be a reduction in the number of vacuolated cells. To evaluate the change more objectively, more than 500 micro-vessels were assessed for their association with vacuolated perivascular cells from 9 sections randomly selected from 3 slides of each animal. Significantly fewer brain capillaries were found to be associated with vacuolated perivascular cells in both MPS/KIiG and MPS/WT groups than those from slides of MPS I controls (p<0.01). Interestingly, the MPS/KIiG mice exhibited significantly less pathologic accumulation than MPS/WT group (p<0.01). Taken together, these results demonstrate that behavioral deficits and CNS pathology can be improved by long-term, supra-physiological IDUA in peripheral blood derived from erythroid cells.

### Example 63

### Implications

This study demonstrated in depth a novel gene therapy approach that leads to extremely efficient, long-term systemic delivery of a non-secreted lysosomal enzyme at supra-physiological levels in the circulation. This approach of restricting transgene expression to maturing erythroid cells may reduce the risk of activating oncogenes in hematopoietic stem cells (HSC) and their progeny, yet take advantage of their robust protein-synthesis machinery for efficient protein production. The data showed that a lysosomal enzyme could be produced at high levels and “secreted” by erythroid cells during in vitro and in vivo definitive erythroid differentiation. Remarkably, with a relatively low vector copy number (0.2-0.3 copy/cell), 4- or 8-fold higher than wild-type IDUA levels were achieved in the blood circulation of primary or secondary MPS I chimeras during the 9 months of observation. These levels are at least 40-fold higher than those observed in MPS I mice fully engrafted with normal donor cells. Considering 5% of normal plasma IDUA levels is therapeutic based on allogeneic BMT experience in MPS I patients (5), one can assess that only 0.3% transduced hematopoietic stem cells would be needed by this erythroid-specific gene therapy approach to achieve similar therapeutic plasma level following autologous transplantation. Thus, this highly efficient, erythroid specific gene expression approach would make it substantially feasible to achieve clinical benefits even with the generally low levels of HSC gene transfer frequency (<1%) commonly obtained in most human HSC gene therapy clinical trials.

Unlike intracellular IDUA polypeptides, the released form of IDUA from normal or enzyme-overexpressing cells appears not to be proteolytically processed and exhibits a unique molecular weight that is not found in cell lysate (18, 24). This study indicates that the IDUA levels were very low in the lysate of uninduced MEL cells (5% of mouse fibroblasts) and declined to negligible levels during erythroid induction, while undetectable levels were found in culture medium. This is not surprising, because the number of active genes decreases dramatically due to global transcriptional repression during definitive erythropoiesis (25). In addition, normal cells are known to release only a small portion of their IDUA, although the molecular and cellular determinants for IDUA release remain undefined. The data further showed in vitro that induced, IDUA-overexpressing MEL cells could release IDUA in a similar pattern, but to a lesser extent than intracellular enzyme production. Moreover, the released form of IDUA is fully functional with normal lysosomal enzyme trafficking, and suitable for uptake by other cells via receptor-mediated endocytosis, resulting in cross-correction of phenotypic defects in cells from MPS I patients. Importantly, experimentation demonstrated, in vivo, that the IDUA produced by erythroid cells could lead to long-term systemic metabolic correction, as well as complete normalization of tissue pathology in all tested peripheral organs of treated MPS I mice.

While proviral integration into hematopoietic stem cells by randomly integrating viral vectors has the potential to provide a life-long therapeutic effect, it also carries the risk of insertional oncogenesis from the strong viral enhancers that can ubiquitously activate transgene expression (26-28). Vector genotoxicity has dampened the clinical success of ex vivo stem cell gene therapy for children with severe X-linked combined immunodeficiency (7, 8) and X-linked chronic granulomatous disease (10). Subsequent studies have demonstrated that the ability of LTR promoter/enhancers to trans-activate genes over large distances in both directions largely attribute to the increased risk of transforming potential of vectors (27, 29). Thus the use of vectors with intact LTRs now has limited clinical utility, even though many LTRs have been shown to provide robust transgene expression with resistance to transcriptional silencing (30), such as the consistent expression from SF promoter observed in this study during erythroid differentiation. As an alternative, promoters from cellular housekeeping genes may provide ubiquitous, multi-lineage transgene expression, and reduce the frequency of transactivating oncogenes. The EFla promoter is one of the strongest such promoters in HSCs tested in vitro and in vivo (31). Yet, by restricting transgene expression to a single lineage, the erythroid specific hybrid promoter evaluated here generated 4-fold higher IDUA plasma levels than those derived from EFla promoter. Moreover, this tissue-specific vector may provide additional safety benefits compared with ubiquitous promoters. First, the possibility of transactivating neighboring genes is limited to a much smaller number of integration sites in transgene-containing progeny of transduced HSCs, reducing the risk of insertional oncogenesis. Second, highly efficient IDUA expression and release by IHK would reduce the demand for high vector copy numbers that are often associated with increased risks of genotoxicity. Lastly, this research demonstrates that IHK-derived transgene expression was predominantly restricted to late stages of erythroid differentiation. Thus, the timeframe for active transcription from the IHK promoter during precursor maturation is relatively brief, approximately 3-4 days (32). This is followed by expulsion of the nucleus as the cells become reticulocytes, which is arguably one of the most radical safety features imaginable.

Several factors may have contributed to the high efficiency of erythroid cell-derived systemic lysosomal IDUA production reported in this study. First, red blood cells are the most abundant blood cells, and are constantly replenished at a rate of more than 2×106 per second under normal hematopoiesis (33). The enormous cell mass and rapid turnover are likely to boost IDUA production at any given time and contribute to the high plasma enzyme levels. Second, Sadelain and colleges have demonstrated the feasibility of introducing long-term secretion of a secreted clotting factor, human factor IX, using a β-globin promoter and its locus control region (16). In this study the following elements were used: a hybrid promoter/enhancer containing the core sequence from human ankryin-1 gene promoter (34), a strong enhancer HS40 variant upstream from human embryonic ζ-globin gene (35), and the intron 8 enhancer of erythroid ALAS gene (36). This promoter has been shown in vivo to drive high erythroid-specific GFP expression, and to retain viral titers due to its relatively small size in comparison to other erythroid promoters (17).

While reprogramming erythroid cells for highly efficient, continuous lysosomal enzyme production in circulation with phenotypic corrections is in itself an important finding, the improvement in brain pathology and behavioral deficit in MPS I mice after long-term peripheral IDUA delivery is one of the most compelling observations of this study. It has been generally believed that the blood-brain barrier (BBB) in the mature brain is largely impermeable to lysosomal enzymes including IDUA; and that the CNS benefits observed in some LSD patients receiving allogeneic BMT treatment early in life (under 2-year old) may be dependent upon diapedesis of donor HSC-derived macrophage-monocytes into the brain (37). Recently, a study performed on mice with another LSD, metachromatic leukodystrophy (MLD), showed that gene marked HSCs overexpressing relatively high levels of the aryl sulfatase A enzyme (ARSA) were far more efficient at reversing the pre-existing CNS deficits (demyelination) than a bone marrow transplant using normal HSCs (38). Higher than normal ARSA levels were achieved in serum by transplantation of transduced HSC (using a LTR promoter). The gene-modified, donor-derived, ARSA-overexpressing microglia cells were proposed to be the exclusive source of ARSA in the CNS. However, it was demonstrated herein that CNS benefit could be obtained when the sole source of IDUA was in the peripheral circulation. One possible reason could be that migrating white cells in the CNS were “super-charged” with IDUA by endocytosis from constant, high enzyme levels in serum before crossing the BBB. It has been suggested that CNS pathology in several MPS conditions (including MPS I) contain an inflammatory component, which encourages more diapedesis than that occurs under healthy condition (39). On the other hand, several studies in some LSD models have shown evidence of partial clearance of CNS storage after multiple infusions of large doses of synthetic corrective enzyme in adult mice (40, 41). Low levels of brain entry were implicated to count for the effects, even though the disappearance of these proteins from serum were reported to be in minutes. More recently, it has been suggested that slowing clearance of the recombinant enzyme from circulation could further improve CNS pathology in MPS VII mice (42, 43). The LV-mediated erythroid-specific gene therapy approach developed here could provide continuously higher than normal IDUA in the circulation with potential life-long CNS therapeutic benefits.

In summary, these results are the first to demonstrate that late-stage erythroid cells, transduced with a tissue-specific LV, not only can produce and release a lysosomal enzyme successfully and continuously at supra-physiological levels in circulation, but also can achieve phenotypic correction in peripheral organs and the CNS of MPS I mice. This approach will break the conundrum of achieving high-efficacy with high-copy numbers, thereby increasing the risk of oncogenesis. This study has important practical implications for treatment of many lysosomal storage diseases involving neurological defects, although the efficacy of this approach in large animal models remains to be assessed. These studies could also open a door for the utilization of red blood cell precursors as a depot for efficient, safer, systemic delivery of non-secreted proteins by ex vivo HSC gene transfer.

### Example 64

### Lentiviral Vector Construction, Packaging and Concentration

Three bicistronic self-inactivating LV were constructed by insertion into a 3^{rd}-generation LV backbone pLV-TW (1) at the Afe I restriction sites (between cppt and WPRE) with EF1 D (GenBank AF403737, 1-1192), or LTR promoter/enhancer from SFFV (2), or an erythroid specific hybrid promoter containing a human ALAS2 intron 8 erythroid specific enhancer, HS40 core element from human alpha LCR and human ankyrin-1 promoter (3). The expression cassette IDUA-ires-GFP, containing human IDUA cDNA (4) and eGFP, was inserted into the Hpa I site. The transfer LVs were packaged by co-transfection of 293T cells with three helper plasmids: p2NRF for gag-pol, pEF1. Rev for Rev and pMD.G for VSVG env function as previously described (1). The potency of viral stocks (typical 10⁸-10⁹ TU/ml) was determined by FACS analysis for GFP⁺ percentage on 293T cells or MEL cells (for LV-KIiG vector) exposed to serial LV dilutions using FACS Canto Flow Cytometer (Becton Dickinson, Lincoln Park, NJ). Less than 30% of GFP⁺ cells were considered reliable for titer calculation when most transduced cells contained 1 copy transgene.

### Example 65

### Cell Line Manipulation

MEL cells were cultured at the concentration of 0.5-2×10⁶/ml in DMEM medium with 10% fetal bovine serum (FBS) and antibiotics. Cells were transduced with each vector stock at 2-3 MOI. To induce erythroid differentiation, MEL cells were subcultured at 106/ml in DMEM containing 20% FBS and 1 mg/ml hexamethylene bisacetamide (HMBA) with medium change every other days for a total of 8 days. To monitor erythroid differentiation, cytospins were prepared at 500 rpm×5 min in a Cytospin® 4 Cytocentrifuge (Thermo Shandon Inc, Pittsburgh, PA) and stained with Wright stain (Harleco EMD).

LCL cell lines were prepared by transformation of PBL from a normal individual or a MPS I patient with Epstein-Barr virus (5). Cells were routinely cultured in RPMI medium with 10% FBS, 2 mM glutamine and antibiotics. All cells were maintained at 37° C. in a humidified atmosphere containing 5% CO2, and were routinely tested for free of Mycoplasma infection.

### Example 66

### Uptake Assay and Lysosomal Staining in LCL Cells.

To evaluate enzyme uptake in enzyme deficient cells, 1×10⁶ LCLmps cells were incubated for 3 hr at 37° C. with 5% CO2 with 1 ml of medium that has been preconditioned by 24-hr culture of MEL-KIiG at Day 7 of induction culture and contained 30 U/ml IDUA enzyme. Control medium, which was preconditioned by 24-hr culture of MEL cells at Day 7 of induction and contained undetectable IDUA, was applied to untreated LCLmps and LCLnormal. To inhibit IDUA uptake, 1 mM mannose 6-phosphate (M6P) (Sigma) was added 30 minutes prior to subculture with enzyme-containing medium, as well as during uptake incubation. Each experiment was performed in triplicate wells.

To study lysosomal morphology change, an aliquot of treated cells described above were washed three times and incubated for 1 hr with 75 nM LysoTracker Red (Invitrogen, NY). After three washing steps with PBS and 1% FBS, we then fixed cells with 4% paraformaldehyde, and followed by cytospin at 500 rpm×5 min in a Cytospin® 4 at 1×105/spot. The slides were then mounted using VECTASHIELD mounting medium with DAPI (Vector Laboratories Inc., Burlingame, CA) and observed using an Olympus inverted fluorescence microscope.

### Example 67

### Isolation, Transduction and Transplantation of Lin-Cells.

To enrich HSC, low-density bone marrow cells were stained with a set of biotinylated antibodies including anti-CD3e, B220, CD4, CD8, CD11b, Gr-1 and Ter119, followed by lineage depletion using anti-biotin microbead-mediated MACS LS column (Miltenyi Biotec Inc). Ex vivo transduction of Lin cells was conducted by culturing cells for a 12-hr prestimulation period in serum-free StemSpan medium (StemCell Technologies) supplemented with 40 μg/ml LDL, 50 ng/ml stem cell factor (SCF), 20 ng/ml thrombopoietin (TPO), 10 ng/ml IL3, 50 ng/ml IL6. Cells were then transduced twice within 24-hr at the presence of 8 μg/ml protamine sulfate. Lin cells were then injected into lethally irradiated (split dosage of 700 and 475 cGy) mice at 10⁵ cells/mouse. All animal procedures were approved by Institutional Animal Care and Use Committee of Cincinnati Children's Hospital Medical Center.

### Example 68

### Immunochemical Staining and Flow Cytometry Analysis

LCL cells were stained with LysoTracker Red (Invitrogen), and evaluated using mounting medium with DAPI (Vector Laboratories Inc.). Fresh bone marrow or CFU-S cells were immunostained with PE-conjugated anti-CD71 and PE-Cy7-conjugated anti-Ter119 (BD Biosciences) as previously described (6), with concurrent staining for 7-amino-actinomycin D (BD Biosciences) to gate out apoptotic cells. Single cell suspensions were analyzed using a FACS Canto with FacsDiva software v6.1 (Becton Dickinson).

### Example 69

### Spleen Colony Forming Unit Assay

CFU-S assay was performed by transplanting 1×10⁵ bone marrow cells from a primary recipient into each irradiated (950 cGy) C57BL/6J mouse. Discrete spleen colonies were collected 12 days after transplantation. Aliquots from each colony were analyzed by enzyme assay for IDUA expression, and by qPCR for copy number analysis.

### Example 70

### IDUA Enzyme Assay.

The catalytic activity of IDUA was measured with a fluorometric enzyme assay as previously described with modifications (4). Cell pellets were homogenized in distill water using Ultrasonic Processor (GE). Aliquots of cleared lysate, plasma or culture medium were incubated with 2.5 mM fluorogenic substrate, 4-methylumbelliferyl (4MU) α-L-idopyranosiduronic acid sodium salt (Toronto Research Chemicals Inc., North York, ON, Canada), together with no sample blank controls in parallel. All samples were assayed in duplicate reactions and each reaction was quantified twice using a Fluorescent plate reader (SPECTRA MAX GEMINIxS from Molecular Devices). Protein concentration was measured by Coomassie blue dye-binding assay (BioRad, Hercules, CA). One unit (U) of enzyme activity is defined as the release of 1 nmol of 4MU in a 1-hr reaction at 37° C. The intracellular IDUA specific activity was calculated as U/mg protein, and extracellular IDUA activity U/ml medium.

### Example 71

### Real-Time Quantitative PCR.

GFP transgene and endogenous murine ApoB were both quantified simultaneously in the same 25 μl reaction by real-time PCR, as described previously with minor modification (7). Genomic DNA was isolated from PBL, BM or CFU-S colonies with Gentra Puregene Blood Kit (Qiagen, Valencia, CA). The multiplex reaction contained 5-20 ng genomic DNA, 200 nM of each GFP primer, 200 nM TaqMan GFP probe, 40 nM of each ApoB primer, 200 nM ApoB probe, and 12.5 μl TaqMan 2× Universal Master Mix (Applied Biosystem). Unknown samples were run in triplicate, and standard samples were in duplicate. A standard curve (ranging from 0.1% to 100%) was established from a series of genomic DNA mixtures (10 ng) of a murine myeloid cell line (32Dp210) with a GFP containing cell line (32Dp210-LNChRGFP) (1 copy per genome as determined by Southern blot analysis). The amplification conditions were 2 min at 50° C. and 10 min at 95° C. for the first cycle, followed by 45 cycles of 95° C. for 15 sec and 60° C. for 1 min.

### Example 72

### Quantification of Urinary GAG.

Urine samples were obtained by bladder palpation. We quantified GAG excretion based on methods previously described (8) with modifications. Briefly, urine aliquots were serially diluted with sodium formate buffer (pH3.0), and mixed in duplicate with freshly prepared 1,9-dimethylmethylene blue (DMB) solution (0.35 uM in sodium formate buffer, pH 3.0). Absorbance of the color reaction was measured at 535 nm within 30 min on DU50 spectrophotometer and compared with standard curve generated with heparan sulfate standard solutions (Sigma). To normalize urine concentration, urinary creatinine was quantified by incubating diluted samples with freshly made picric acid/sodium hydroxide solution (10% saturated picric acid and 0.09M NaOH) for 20 min, measuring absorbance at 535 nm and calculated using standard curve established with creatinine reference solutions (Sigma).

### Example 73

### Chemical Staining and Pathology Evaluation.

At the end of observation period, mice were euthanized by intraperitoneal administration of an overdose of sodium Nembutal (Abbott Laboratories). After blood collection and removal of hind legs for marrow harvest, each mouse was perfused transcardially through the aorta with cold normal saline briefly, and followed by 4% paraformaldehyde. Tissue samples were fixed by 2% glutaraldehyde in 0.175M sodium cacodylate buffer (pH7.4) at 4° C. The tissue is then treated with 1% osmium tetroxide, washed in 0.175M sodium cacodylate buffer, dehydrated by a graded ethanol series and embedded in LX112. Sections (0.5-1 μm) were prepared and stained with 1% Toluidine blue in 1% sodium borate, followed by examination for the presence of pathologic storage vacuoles. Two animals per groups were analyzed with 6 sections randomly selected from 3 slides for each organ. For brain pathologic scoring, cells containing <5 cytoplasmic vacuoles were considered as normal while those with >30 vacuoles as positive. More than 500 micro-vessels were scored for each animal from 9 sections randomly picked from 3 slides. The mean of scoring data from 6 slides is shown for each group.

### Example 74

### Behavioral Test.

The repeated open-field test was performed 5 months after BMT at the age of 7-months as described previously (9). The open-field apparatus (60×60 cm) consisted of a white Plexiglas box with 25 squares (12×12 cm) painted on the floor (16 outer and 9 inner). Briefly, the mouse was placed in one of the four corners of the apparatus and allowed to freely explore the whole field for 5 min. Activity was monitored and quantified for ambulation (number of inner and outer squares crossed), rearing frequency and time spent grooming by two observers who did not know the genotype or treatment of the animal during testing. Each mouse was tested for three repeated trials with 30-minute inter-trial intervals.

### Example 75

### Statistical Analysis.

All quantitative assays were performed in duplicate or triplicate from at least two individual experiments. Data are presented as mean±standard deviation unless specified. Comparisons between two groups were performed using two-tailed Student t-tests. P values of less than 0.05 were considered as statistical significance.

### Example 76

### Generally—

Sickle cell anemia (SCA) results from a point mutation in the-globin gene ((3s), resulting in sickle hemoglobin (HbS). HbS polymerizes upon deoxygenation resulting in sickle-shaped RBCs that occlude microvasculature. Patients with SCA have intermittent acute vascular occlusions and cumulative organ damage, reducing the life span to 42 to 58.5 years. Besides sickling, excessive hemolysis and a state of chronic inflammation exist. SCA patients account for approximately 75,000 hospitalizations per year, resulting in an estimated annual expenditure of $475 million dollars in the United States alone. Worldwide, SCA is second only to thalassemia in incidence of monogenic disorders, with more than 200,000 children born annually in Africa.

Current therapies include supportive care for episodic sickling, chronic transfusions with iron chelation, and hydroxyurea to induce fetal hemoglobin (HbF). These therapies impact disease morbidity, but their effectiveness is variable and dependent on compliance to an indefinite treatment regimen. A matched allogeneic hematopoietic stem cell (HSC) transplantation is curative, but restricted by the availability of matched related donors5 and has potential serious complications. A meta-analysis of 187 SCA transplantations shows 6% to 7% conditioning-related peritransplantation mortality, 7% to 10% acute rejection, and 13% to 20% chronic graft-versus host disease (GVHD) in recipients.

Gene therapy of autologous HSCs followed by transplantation could result in a one-time cure, avoid adverse immunologic consequences, and not be limited by availability of donors; it may also not require myeloablative-conditioning regimens, and thereby have lower toxicity. The amount of HbF/anti-sickling globin required to correct SCA via a transgene is unknown.

Expression of HbF postnatally can be therapeutic, as is evident by the protective effect of HbF in neonatal sickle RBCs and in patients with hereditary persistence of HbF and SCA. The proportion of genetically corrected HSCs, the amount of exogenously expressed HbF, and the proportion of F cells that will correct the pathophysiology are unknown. Complete correction of human thalassemia major in vitro, and in xenografted mice in vivo, with a lentivirus vector carrying the β-globin gene and locus control region (LCR) elements has been demonstrated. In this report, this β-globin lentivirus vector was modified to encode γ-globin exons and murine sickle HSCs were transduced. Functional correction was characterized first, with a careful and detailed quantification of RBC sickling, half-life, and deformability, with sickle to normal transplantations and high HbF production to define parameters of correction. Next, using reduced-intensity conditioning and varying the percentage of transduced HSCs, transplantations were performed on sickle mice with significant organ damage and demonstrate the proportions of (1) genetically corrected HSCs, (2) HbF, and (3) F cells, and (4) percentage of HbF/F cell required for correction of the sickle RBC and amelioration of organ damage in SCA.

### Example 77

### Vector

It has been demonstrated that a β-γ-globin hybrid gene carrying lentivirus vector, 18H β/γW, 11 expresses high γ-globin mRNA in erythroid cells expressing “adultlike” globins. All β-globin coding sequences were changed to γ-globin using site-directed mutagenesis and the γ-β-globin hybrid gene, and LCR elements were cloned in reverse orientation to the viral transcriptional unit to generate sGbG lentivirus vector. Virus was made with cotransfection of 293T cells.

### Example 78

### Murine HSC Enrichment

Bone marrow from 6- to 20-week-old BERK sickle mice was harvested and lineage depleted with biotinylated CD5, CD8, B220, Mac-1, CD11b,Gr-1, and TER-119 antibodies and magnetic beads. The bead-free cells were stained with antibodies to Sca-1, c-kit. Cells that were 7-AAD-, Lineage-, c-kit then Sca-1± (LSK cells) were sorted on FACSVantage (BDBiosciences). All experiments using Berkeley transgenic sickle mice and C57/BL6 mice were performed according to protocols approved by the Cincinnati Children's Hospital Medical Center.

### Example 79

### Gene Transfer and Bone Marrow Transplantation

Myeloablative transplantations were performed from BERK3C57B1/6 mice because of ease of transplantation and ready availability of normal recipients (9.5^{+/-}0.6 weeks old) after 11.75 Gy radiation. Radiation control experiments showed that BERK mice receiving 8 to 9 Gy radiation survived without receiving LSK cells; and the lethal dose was lower than in C57B1/6 mice. BERK mice receiving more than 10.5 Gy died when no LSK cells were given; those given LSK rescue survived long term. BERK mice are difficult to breed in large numbers at a given time, therefore 2 mice/radiation dose level were to determine the sublethal dose. All BERK recipients (12.9±0.4 weeks old) received 3 peritransplantation RBC transfusions (days 1-7). Organ pathology in BERK recipients 1 year after transplantation was compared with 12-week-old BERK mice that did not undergo transplantation. The radiation was higher than classical reduced intensity radiation dose of 4 Gy to allow a large degree of donor HSC chimerism. A range of MOI was used to vary the proportion of transduced donor HSCs in the graft. LSK cells were prestimulated overnight and transduced twice at an MOI of 30 for BERK3C57BL/6 transplants and MOI of 30 to 100 for BERK-BERK transplants for 22 to 24 hours; 10,000 to 24,000 LSK cells and untransduced LK cells were cotransplanted into recipient C57BL/6 or BERK mice.

### Example 80

### Copy Number Analysis

Copy number analysis was done on genomic DNA by real-time polymerase chain reaction using primers and probes described previously.

### Example 81

### Hematologic Analysis

Hematologic analysis was obtained on Hemavet 950FS (Drew Scientific) under mouse settings. Reticulocyte analysis was performed as follows: 0.1 μL blood and 200 μl, BD Retic-COUNT Reagent were mixed (Becton Dickinson), incubated at room temperature for 30 minutes, and analyzed by fluorescence-activated cell sorting (FACS).

### Example 82

### Hemoglobin Analysis

Hemoglobin electrophoresis was performed on cellulose acetate plates, as described previously. Ion exchange high-performance liquid chromatography (HPLC) was performed with an Alliance 2690 HPLC machine (Waters) using a PolyCATAcolumn (item no. 3.54CT0510; Poly LC Inc).

### Example 83

### Red Blood Cell Functional Analysis

Irreversibly sickled cells (ISCs) were enumerated by scoring 500 RBCs in consecutive fields. Graded deoxygenation was performed using tonometry. RBC deformability was determined using a laser-assisted optical rotational cell analyzer (LORCA; RR Mechatronics).

### Example 84

### RBC Half-Life

Mice were injected with 3 mg Sulfo-NHS biotin (Sigma) in 300 μl, PBS as 2 separate injections 1 hour apart; 2 to 5 μI, blood was drawn at serial times, and stained with APC-Cy7-conjugated streptavidin.

### Example 85

### Histology

Spleen, liver, bones, brain, and kidney were harvested and placed in 5 mL of 10% formalin. Paraffin blocks were sectioned and stained with hematoxylin and eosin.

### Example 86

High HbF after Gene Therapy and Myeloablative Transplantation Corrects SCA

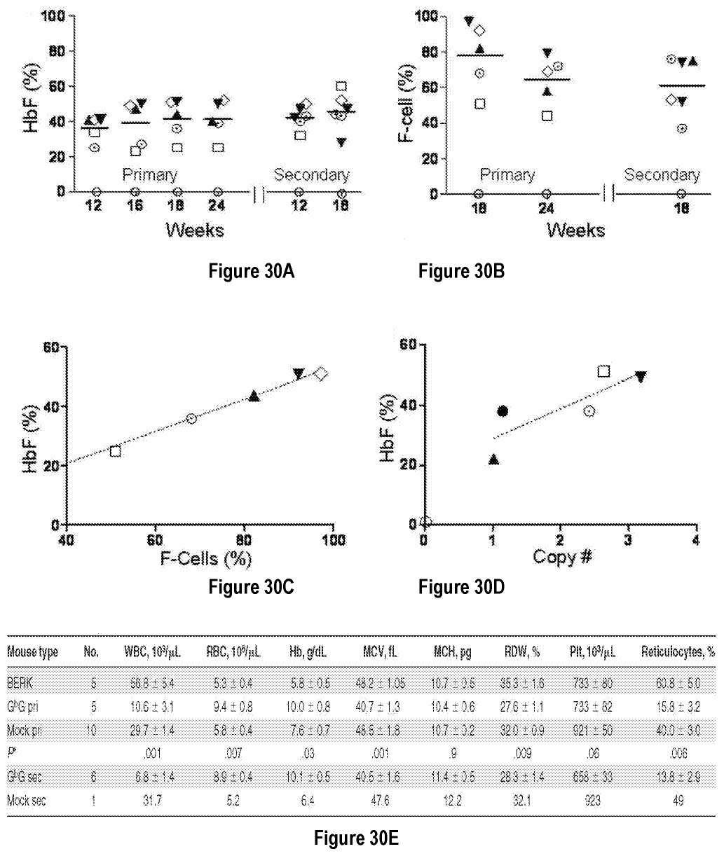

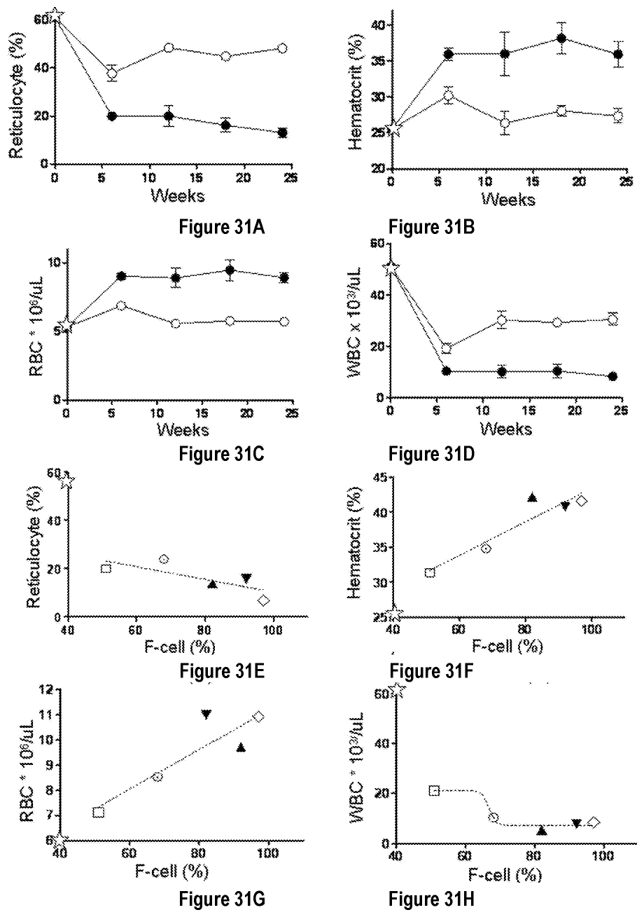

The sGbG vector carries γ-globin exons and β-globin noncoding and regulatory regions. Based upon a previously studied sBG vector, which expresses high levels of human β-globin, sGbG-transduced LSK cells from Berkeley sickle (BERK) mice were transplanted into lethally irradiated (myeloablated) normal C57B1/6J mice (termed GbG mice). Mock transductions on BERK LSK cells from the same bone marrow pool followed by transplantation resulted in mice with SCA. The majority of RBCs in GbG mice expressed HbF. Only GbG mice with 100% donor (HbS⁺) RBCs, with no evidence of residual recipient murine hemoglobin by electrophoresis and HPLC, were analyzed for hematologic, functional, and pathologic analysis. GbG mice with a small proportion of recipient murine RBCs, were used only to assess HbF/vector copy and frequency of transduced HSCs. The percentage of HbF (HbF/HbS+HbF) in blood, quantified by FACS, was approximately 40% in primary mice followed for 6 months and in secondary recipients followed for 7.5 months (FIG. 30A). Two-thirds of RBCs were F cells; their proportion was also stable in primary and secondary recipients (FIG. 30B). The proportion of F cells and vector copies correlated with HbF (FIG. 31C-D). Taken together, these data show significant HbF expression from the s G^{b}G vector in the majority of RBC with stable long-term expression.

### Example 87

### High Levels of HbF Result in Sustained Hematologic Correction

FIG. 30E shows improvement of hematologic parameters in GbG mice. The proportion of reticulocytes decreased from approximately 50% in mock mice to approximately 15% in G^{b}G mice (P<0.005; FIG. 31A). There was correction of anemia by 12 weeks, which persisted throughout the post transplantation period (FIG. 31B-C).

High white blood cell (WBC) counts in humans with SCA and BERK mice reflect the baseline inflammation in this disease. WBC returned to normal levels in GbG mice (FIG. 31D; FIG. 30E).

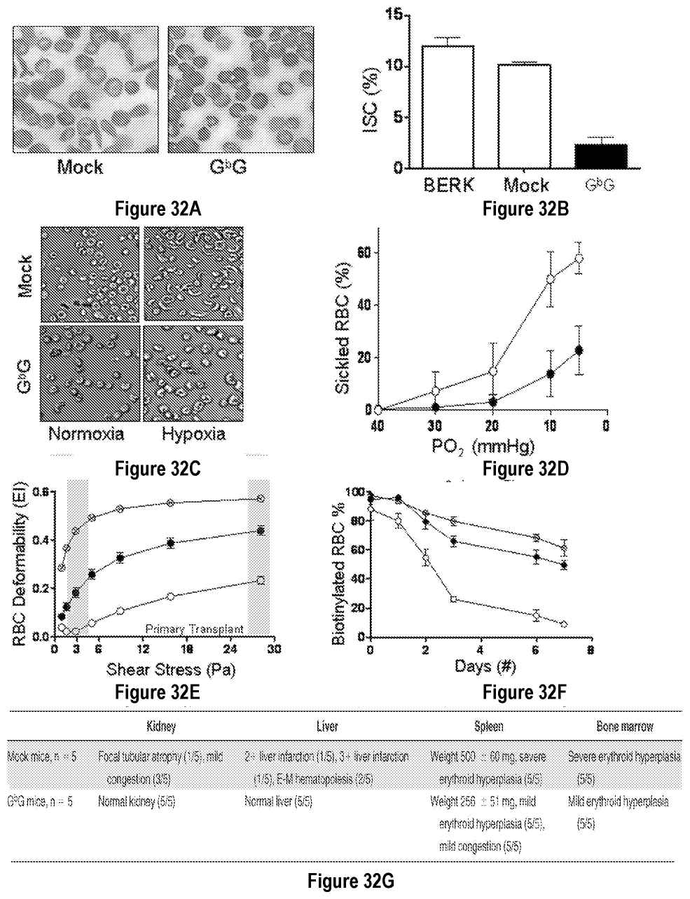

Notably, WBC counts were lower in the mock mice compared with BERK mice that did not undergo transplantation, likely because in the former, sickle HSCs were transplanted into a normal “noninflamed” C57/BL6 background. Indeed, 6 weeks after transplantation, WBC counts in mock group of mice were nearly normal, then gradually rose to high levels seen in SCA (FIG. 31D) Overall, hematologic parameters showed marked improvement to near normal levels, and improvement was stable over a prolonged period in primary and secondary GbG mice. The degree of correction correlated with the proportion of F cells (FIG. 31E-H) and HbF (data not shown). High levels of HbF improve the functional parameters of RBCs in sickle mice. (1) Sickling: The irreversibly sickled cells (ISCs) were significantly reduced to 2.3% plus or minus 0.7% in GbG mice, compared with 12% plus or minus 0.8% in BERK controls and 10.2% plus or minus 0.3% in mock mice (FIG. 32A-B). Deoxygenation of blood from a representative GbG mouse shows a dramatic reduction in sickling (FIG. 32C). A systematic quantification showed a marked decrease in the proportion of sickle RBCs in GbG mice with increasing hypoxia (FIG. 32D). (2) RBC membrane deformability: Normal RBCs deform readily at low shear stress (3 Pascals [Pa]), representative of shear stress in small vessels. Sickle RBCs have relatively rigid membranes with remarkably reduced deformability even at high shear stress (28 Pa; representative of shear stress in large vessels). There was markedly improved deformability of RBCs of GbG mice, although it did not achieve normal levels (FIG. 32E). This may reflect the proportion of circulating sickle RBCs that did not contain HbF. (3) RBC survival: Survival of human sickle RBCs is an order of magnitude less than normal RBCs. The time to 50% reduction (half-life) in G^{b}G and mock/BERK sickle mice was measured. The overall survival of the GbG RBCs was markedly improved, with the time to 50% reduction approximately 4 times longer in RBCs from G^{b}G mice compared with BERK or mock mice (FIG. 32F). (4) RBC hemolysis: RBC hemolysis detected by measuring lactate dehydrogenase (LDH) in blood was reduced from 2706 plus or minus 148 mg/dL in mock mice to 1286 plus or minus 345 mg/mL in GbG mice (n=5; P<0.004).

### Example 88

High Levels of HbF Prevent Chronic Organ Damage Associated with SCA

Bone marrow, spleen, liver, and kidneys at 24 weeks showed complete prevention of organ pathology. There was reduced erythroid hyperplasia in bone marrow and spleen, decreased spleen size, and preservation of the splenic follicular architecture, compared with obliterated follicular architecture from the severe erythroid hyperplasia in mock mice. The focal tubular atrophy and segmental glomerular infarction seen in mock mice were absent in the GbG mouse kidneys. Infarctions and extramedullary hematopoiesis seen in livers of mock mice were absent in livers of GbG mice (FIG. 32G summarizes the data in all groups of mice). Overall, except for a mild erythroid hyperplasia no organ pathology was observed in the GbG mice.

### Example 89

### High HbF Expression Improves Survival of Sickle Mice.

The life span of BERK sickle mice is significantly reduced, as in humans with SCA before modern treatment. Kaplan-Meier survival curves showed a 100% survival of the G^{b}G mice at 24 weeks, in contrast to 20% survival in mock mice (n=14, P<0.001).

### Example 90

### Minimal Parameters Required Correction of SCA

Myeloablative conditioning allows noncompetitive repopulation of gene-corrected donor HSCs, resulting in high transgene-modified HSC engraftment and transgene expression. It was hypothesized that high levels γ-globin expression achieved by myeloablative conditioning may not be necessary for correction, and if so, would reduce transplantation-related morbidity.

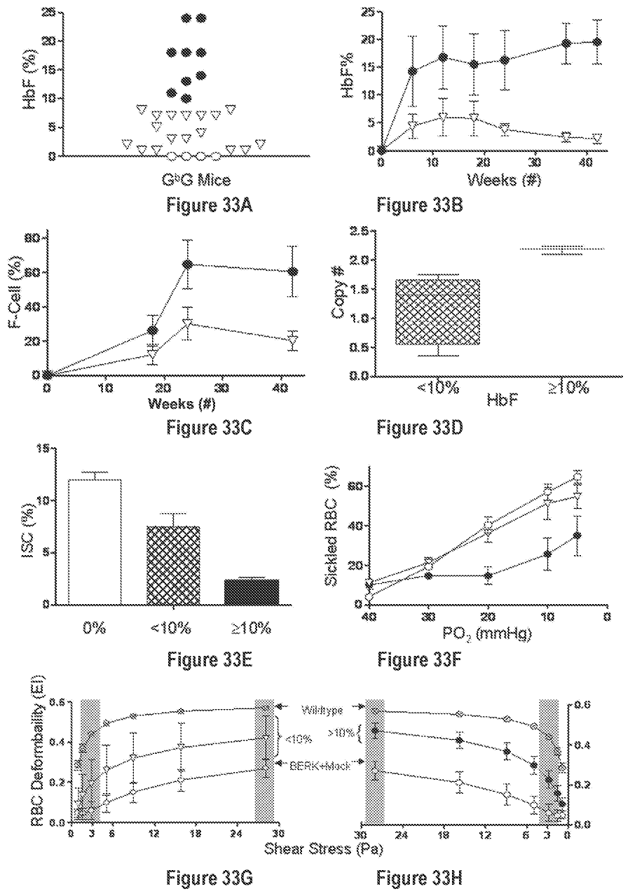

Reduced-intensity transplantation was accomplished by transplanting gene-modified BERK LSK cells into sublethally irradiated, but with significantly high radiation dose, BERK mice. The proportion of transduced HSCs and vector copy/cell in the graft was varied by transducing LSK cells with at a range of MOI (30-100). Since the half-life of BERK RBCs was 1.5 to 2 days (FIG. 33G-H), mice were transfused in the peritransplantation period and analyzed after 12 weeks. Three serial experiments were carried out with mice followed for 1 year. G^{b}G mice were analyzed by separating them into 3 groups based upon percentage of HbF at 18 weeks: HbF=0% (mock, n=4), HbF less than 10% (termed G^{b}G<10; n=17), and HbF of 10% or more (termed G^{b}G>10; n=9); (FIG. 33A). The cutoff at 10% HbF was selected as this appeared to be a threshold level of HbF that reflected correction of disease: G^{b}G<10 mice showed a higher mortality and inconsistent hematologic correction, compared with G^{b}G>10 described in the following paragraph. The mouse numbers in the groups changed with time primarily due to the increased mortality related to SCA in mice with no/low HbF. The G^{b}G≥10 group of mice had 16% (±1.2%), 17% (±1.8%), and 21% (±2.3%) HbF, whereas the G^{b}G<10 group of mice had 5% (±1.4%), 4% (±0.6%), and 4% (±0.5%) HbF at 12, 18, and 24 weeks, respectively, that was stable up to 1 year (FIG. 33B). F-cell repopulation was significantly higher in G^{b}G>10 mice (65%±14%) compared with G^{b}G<10 mice (30%+9.4%; (FIG. 33C). G^{b}G>10 mice had 2 to 2.5 vector copies/cell, whereas the G^{b}G<10 mice had 1.4 copies/cell (FIG. 33D).

### Example 91

Hematologic Improvement Occurred with Reduced-Intensity Transplantations

Hematologic parameters stabilized at 18 weeks, due to persistent transfused RBCs in the early post transplantation period. There was a significant improvement in hematologic parameters in the G^{b}G>10 group of mice (FIG. 32G), in contrast to a small and inconsistent improvement in G^{b}G<10 mice.

### Example 92

Improvement in RBC Function Occurs with Reduced-Intensity Transplantations

Sickling: There was a very significant reduction in ISCs in GbG>10 mice (P<0.005) and a small, but significant reduction in ISCs in G^{b}G<10 mice compared with mock/BERK controls (P<0.05, FIG. 33E). RBCs from G^{b}G>10 mice showed reduced sickling when exposed to graded hypoxia, compared with RBCs from G^{b}G<10 or mock/BERK mice (n=20, P<0.01; FIG. 33F). In contrast, there was no significant difference in sickling between GbG<10 and mock/BERK mice. (2) RBC membrane deformability: Surprisingly, despite similar degree of sickling with hypoxia in RBCs from G^{b}G<10 mice and mock/BERK mice, there was slight improvement in RBC deformability in the G^{b}G<10 mice. However, these differences were not statistically significant from the mock/BERK mice due to the high variance (FIG. 33G). In contrast, there was a consistent significant improvement in RBC deformability in G^{b}G>10 mice (P<0.001, FIG. 33H). The deformability pattern suggested improved RBC flow through large vessels and microvessels. (3) RBC survival: RBC half-life of BERK mice was 1.5 days. RBCs of GbG mice with 1%, 3%, and 7% HbF had a slightly higher half-life (2 days). Two G^{b}G mice with 18% HbF showed an RBC half-life of 6 days, a 4-fold increase, similar to that seen in mice carrying 40% HbF in the myeloablative transplantation model.

Taken together, the sGbG vector resulted in significant and consistent hematologic and functional correction of SCA, when the HbF production exceeded 10% of the total hemoglobin. Notably, the improvement in phenotype was comparable with that achieved with myeloablative conditioning.

### Example 93

Remarkable Improvement in Organ Pathology when HbF Concentrations Exceed 10%

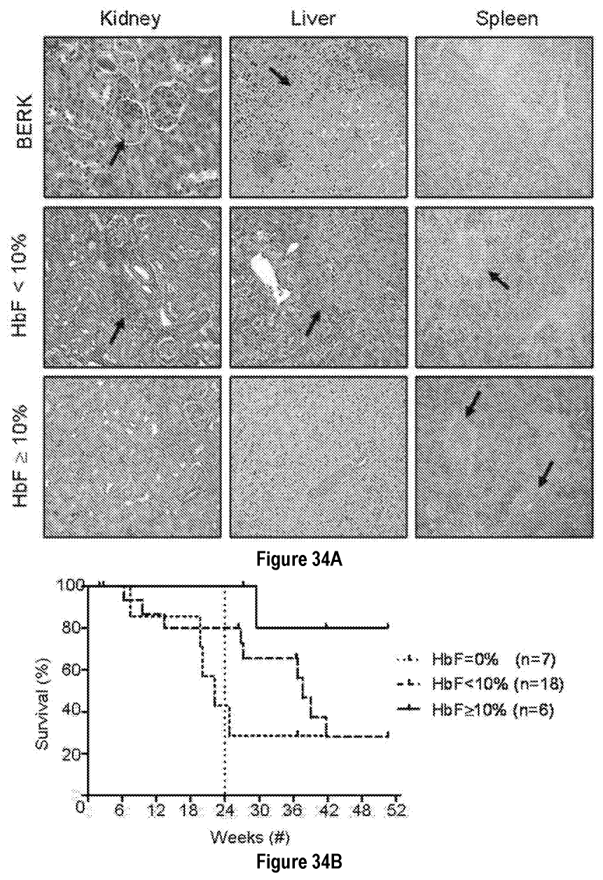

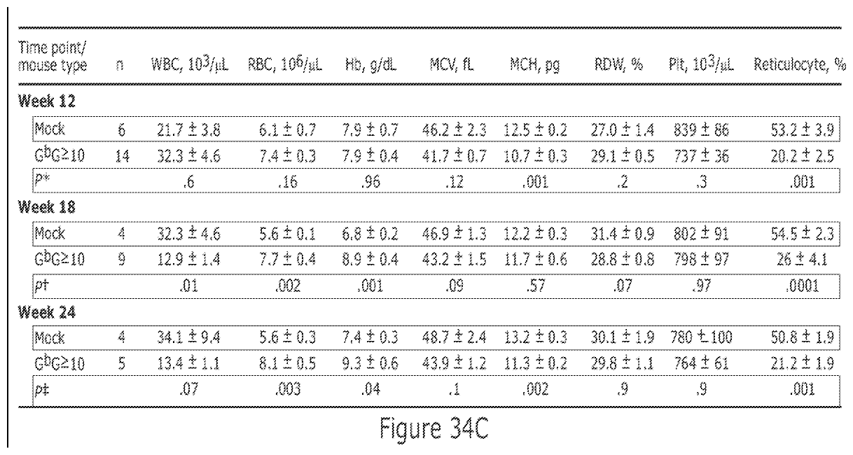

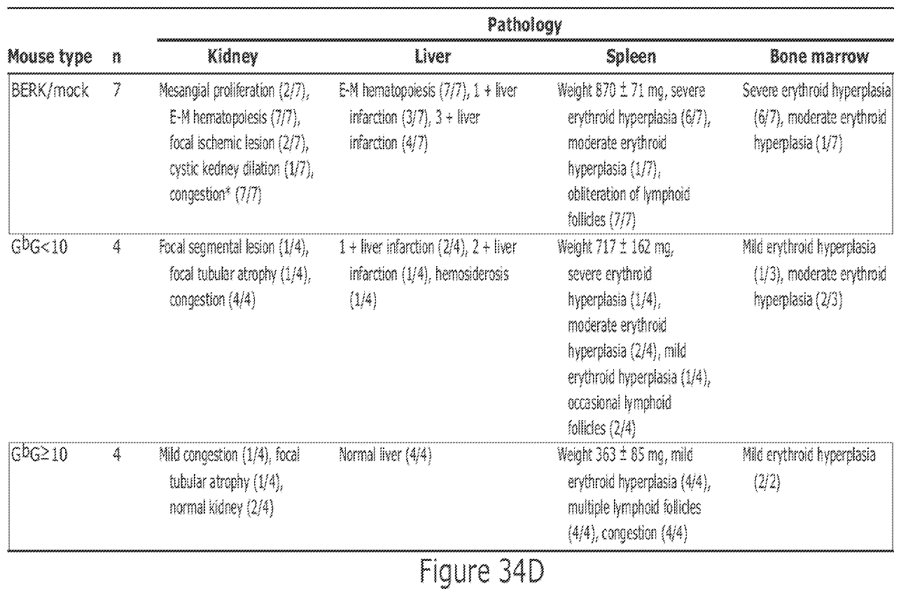

One unique feature of this BERK-BERK transplantation model was presence of significant sickle pathology in recipients at the time of transplantation (determined using BERK controls of comparable age as recipient mice when they underwent transplantation). Therefore, the potential for reversal of organ pathology after gene therapy could be assessed. Organ pathology in the surviving mice at approximately 50 weeks after transplantation was compared with 3-month-old BERK mice that did not undergo transplantation (FIG. 34A; FIG. 34C). The G^{b}G<10 group of mice showed slight improvement in organ pathology: There was a slight reduction in spleen weight (717±162 mg in G^{b}G<10 vs 870±71 mg in BERK/mock mice; P value, NS). Bone marrow and spleens showed moderate to severe erythroid hyperplasia; livers had infarctions and extramedullary hematopoiesis; and the kidneys showed occasional focal segmental lesions, focal tubular atrophy, and vascular congestion (FIG. 34D). In contrast, a dramatic reversal of organ pathology was seen in G^{b}G>10 mice: there was a 50% reduction in spleen weight to 363 plus or minus 85 mg, preservation of splenic follicles, and mild erythroid hyperplasia in bone marrow and spleen. Remarkably, no liver infarctions and no kidney pathology were detected, except in one mouse with a single focus of focal tubular atrophy. Overall, G^{b}G>10 mice showed correction of organ pathology. The lack of organ pathology in GbG mice at 15 months of age compared with 3-month-old BERK controls demonstrates that gene therapy with the s G^{b}G vector in a reduced-intensity transplantation setting prevents any further organ damage, and the existent organ damage at the time of transplantation probably reverses from regeneration.

### Example 94

### Survival

There was a significant improvement in overall survival in the GbG>10 mice compared with G^{b}G<10 or mock mice (FIG. 34B; P<0.05). Indeed, at 24 weeks, survival of the G^{b}G>10 mice was comparable with survival in mice with approximately 40% HbF in the myeloablative transplantation model that were followed for 24 weeks. There was some improvement in early survival in G^{b}G<10 mice compared with mock mice (P<. 05). However, by 1 year, there was no difference in survival of G^{b}G<10 mice over mock mice.

### Example 95

### F Cells and HbF F Cell Critical for Improved RBC Survival and Correction of SCA

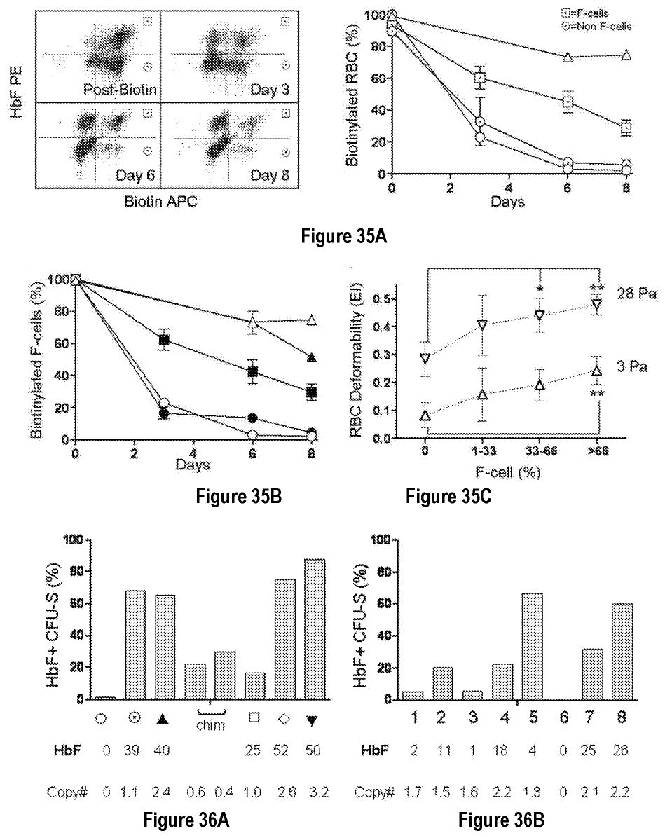

Using biotin surface labeling and intracellular HbF staining, the survival of F cells and non-F cells was studied in the same animal, which allowed quantification of the HbF/F cell necessary for improved sickle RBC survival and deformability. F cells showed a selective prolonged survival, as anticipated (FIG. 35A). The average HbF/F cell 20 in GbG mice in the BERK3C57B1/6 model was 64% (in these mice, HbF was 41%±5%, F cells were 64%±6%). In the reduced-intensity transplantation model, GbG 10 mice had 32% HbF/F cell (in these mice HbF was 21%±2%, F cells were 65%±14%), and G^{b}G<10 mice had 13% HbF/F cell (HbF, 4%=0.1%; F cells, 30%±9.4%). Note that G^{b}G mice in the myeloablative model and G^{b}G>10 mice had similar F-cell repopulation (64%-65%), suggesting that 32% HbF/F cell was sufficient to correct the sickle phenotype. However G^{b}G<10 mice with 13% HbF/F cell and 30% F cells had inconsistent and insignificant amelioration of the disease phenotype.

Therefore, the half-life of F cells in mice was determined, grouped by the percentage of HbF/F cell. GbG mice with low (16%; n=2), intermediate (33%; n=4), and very high (89%; n=2) HbF/F cell was injected with biotin and followed by periodic blood sampling. It was determined that mice with low HbF/F cell had no improvement in RBC half-life over BERK controls (FIG. 35B), those with 33% HbF/F cell had a 3- to 4-fold improvement in half-life, and mice with very high amounts of HbF/F cell showed RBC survival similar to normal mice. These data demonstrate that if one-third of the hemoglobin within a sickle RBC is HbF, there is significant improvement in RBC survival. Mice with these levels of HbF/F cell showed approximately 65% F cells, more than 10% HbF.

To confirm the impact of percentage of circulating F cells on overall RBC deformability, mice from both the myeloablative and reduced-intensity experiments (n=34) were grouped into 3 groups: mice with less than 33% circulating F cells, 33% to 65% F cells, and 66% or more F cells and measured RBC deformability. Only data from the low (3 Pa) and high (28 Pa) shear rates are plotted in FIG. 6C. Mice with more than 66% F cells had a highly significant improvement in RBC deformability at both high and low shear stress (P<0.01). Mice with 33% to 66% F cells had significantly improved RBC deformability only at high shear stress (P<0.05). Mice with less than 33% F cells showed inconsistent improvement in RBC deformability at low or high shear stress, which was not significantly different from mock controls. These data quantify the critical amount of HbF/F cell, the proportion of F cells, and overall HbF that are necessary for correction of sickle cell disease.

### Example 96

### Proportion of Transduced HSCs Required for Phenotypic Correction

The proportion of HSCs transduced with s G^{b}G in G^{b}G mice was analyzed by the secondary spleen colony-forming unit (CFU-S) assay performed at 6 months in both models (FIG. 36A-B). Bone marrow aspirates were performed at 6 months in the BERK-BERK mice that were followed for 1 year. The proportion of transduced CFU-S's was determined by HbF expression. It has been previously shown that all vector-positive CFUs express the transgene in an identical vector that encodes β-globin. G^{b}G mice in the myeloablative conditioning group had 16% to 87% s G^{b}G-transduced CFU-S's (average HSC transduction was −50%), and those in the reduced-intensity group had 5% to 60% transduced HSCs (average HSC transduction was −30%). It is to be noted that in the reduced-intensity model, HSC transduction is overestimated, secondary to the higher mortality of G^{b}G<10% mice in the first 6 months.

Importantly, 3 mice with 16%, 20%, and 22% transduced CFU-S's had more than 10% HbF (HbF was 20%, 11%, and 18%, respectively) and showed complete phenotypic correction. A vector copy number analysis was performed concurrently at 24 weeks on bone marrow cells and showed 1 to 3 copies/cell and 1 to 2.5 copies/cell in GbG mice that underwent transplantation using the myeloablative conditioning and reduced-intensity conditioning models, respectively. When corrected for HSC transduction, there were 1.5 to 5 vector copies/cell.

### Example 97

### Transduction of Human CD34+ Cells

The percentage of gene-modified HSCs necessary for effective gene therapy is critical in this disease. In vitro studies on SCA marrow can be done only on a small scale, and would read out correction in progenitors, not HSCs. HSC correction was shown in humanized models of SCA with long-term analysis. The extremely limited numbers of RBCs produced from injecting human thalassemia bone marrow CD34⁺ cells are prohibitive for studies on sickling. Therefore, lentivirus transduction into normal human CD34⁺ cells was optimized for a preclinical scale-up, using a GFP lentivirus vector and the severe combined immunodeficient (SCID)-repopulating assay. Granulocyte colony-stimulating factor-mobilized peripheral blood CD34±cells transduced with a GFP lentivirus vector were transplanted into nonobese diabetic (NOD)/LtSz-scid IL2Rymill (NOG) mice. Here, mock mice were those that received a transplant of untransduced CD34+ cells immediately after selection, as controls for the effect of transduction on engraftment and clonogenicity. At 6 weeks, CFUs were plated from bone marrow derived from NOG mice, and 36 individual CFUs/mouse were analyzed for the percentage of gene-marked colonies. The 18-hour transduction did not affect engraftment or clonogenicity (data not shown). A 77% gene transfer on average was observed in the SCID-repopulating cell assay, similar to previous data in human thalassemia CD34+ cells.

The data from this study indicates that lentiviral delivery human γ-globin under β-globin regulatory control elements in HSCs results in sufficient postnatal HbF expression to correct SCA in mice. The amount of HbF and transduced HSCs was then de-scaled, using reduced-intensity conditioning and varying MOI, to assess critical parameters needed for correction. A systematic quantification of functional and hematologic RBC indices, organ pathology, and life span were critical to determine the minimal amount of HbF, F cells, HbF/F cell, and gene-modified HSCs required for reversing the sickle phenotype.

Results indicate the following: (1) Amelioration of disease occurred when HbF exceeded 10%, F cells constituted two-thirds of the circulating RBCs, and HbF/F cell was one-third of the total hemoglobin in RBCs; and when approximately 20% sGbG modified HSCs repopulated the marrow. (2) Genetic correction was sustained in primary or secondary transplant recipients followed long-term. (3) There is a method of determining minimum HSC chimerism for correction of a hematopoietic disease in an in vivo model, which would contribute to design of cell dose and conditioning regimens to achieve equivalent genetically corrected HSCs in human clinical trials.

One novel aspect of this study is that it addresses, for the first time, the gene dosage and the gene-modified hematopoietic stem cell dosage required for correction of a genetic defect. Expressing a tremendous amount of fetal/antisickling hemoglobin will undoubtedly correct disease, as has been shown by others, but is not practically possible in a clinical setting. As an example, an initial gene therapy for adenosine deaminase (ADA) deficiency was performed using no conditioning, and was not therapeutic, even though few gene-marked stem cells engrafted, and a selective advantage to gene-corrected lymphocytes was evident upon withdrawal of ADA. In a subsequent trial, 4 mg/kg busulfan was used before transplantation, as conditioning, resulting in adequate gene-corrected stem cell dose and gene-modified T cells. Although these pioneering studies provided us with invaluable information, they underscore the critical importance of determining thresholds for genetic correction before embarking on clinical studies.

Although disease has been corrected at 1 to 3 copies/cell, the present study indicates that the percentage of transduced stem cells in this setting of lethal irradiation/transplantation is very high (average HSCs transduced are 50%, as analyzed by a stringent secondary CFU-S assay). This level of HSC transduction would likely not be achieved in the clinical setting unless myeloablation is performed.

Therefore a novel model (BERK to BERK transplantation) was developed to address the minimal gene transfer needed, and answer questions of correction of SCA in a mouse with significant sickle pathology at 12 weeks of life (FIG. 34). Notably, a sickle to normal myeloablative transplantation, used by other groups showing correction of SCD, is a disease prevention model, where there was no underlying pathology at time of transplantation. The present studies show that repair of preexisting pathology can occur, if genetic correction results in more than 10% HbF.

BERK mice have some degree of thalassemia. Therefore one concern in using this model for genetic therapy studies for sickle cell anemia is that correction of thalassemia would obscure

improvements made by the antisickling effects of HbF. Surprisingly no significant change in MCH in G^{b}G<10 or G^{b}σ>10 mice, including mice with HbF/F cell as high as 89% were seen (as disclosed herein). These results were surprising, but showed that the correction of sickling in RBCs was not secondary to correction of thalassemia, as seen in murine thalassemia model, where increasing MCH was seen with increases in HbF of 4% or higher. Conceivably, HbF is produced at the expense of HbS.

Although BERK mice exclusively carry human hemoglobin, the total hemoglobin in the mouse RBCs is one-third of a human RBC. Therefore, HbF and HbF/F cell were expressed as a percentage, rather than in absolute amounts, to best compare murine data to human. An increase of HbF from 3.6% to 13.6% has been shown to reduce acute sickle events in patients on decitabine. Similar improvement in sickle events occurs with 25% or more HbF/F cell in patients responsive to hydroxyurea. Data presented here, indicating improvement with 33% HbF/F cell, is concordant with these reports, but more closely resemble RBCs in infants with SCA, where less than one-third HbF/F cell at 10 to 12 months is considered a threshold for intracellular sickle polymerization. The most remarkable effect of γ-globin production with the s G^{b}G vector was a dramatic absence of chronic organ damage and an improved survival of the sickle mice when HbF exceeded 10%. Patients with high HbF have an improved survival, confirmed by the multicenter study on hydroxyurea. HbF expressed from the s G^{b}G vector was comparable with, or even better than, effective hydroxyurea treatment. The potential of a one-time correction, where responsiveness to hydroxyurea and compliance to daily life-long administration would not be limiting factors, would be a tremendous advantage of gene therapy. Indeed, we did not anticipate we would get the same conclusion with gene therapy, as derived from collective knowledge on (1) transgenic mice, in which every RBC has the same amount of HbF although we were imposing HbF on SS RBCs; (2) chimeric transplantations, in which normal amounts of HbA-producing RBCs (AA RBC) are present mixed with SS RBCs17,37,38; or (3) SCD patients on hydroxyurea, in whom macrocytosis induced by hydroxyurea would dilute HbS and reduce the threshold for sickling. A much higher threshold of genetically corrected sickle HSCs necessary for F-cell repopulation and correction of SCA phenotype was expected, as HbF was exogenously imposed into a sickle cell with normal amounts of HbS. Notably, despite these distinct differences in transgenics/chimeras, conclusions were similar with exogenous γ-globin expression: Indeed expressing exogenous HbF in RBCs at concentrations from 33% to as high as 89% resulted in no significant increase in MCV or MCH, yet corrected sickling. This data suggests that genetic delivery of HbF decreases endogenous HbS.

The percentage of transduced HSCs in the setting of lethal irradiation/transplantation is very high (50% on average, as analyzed by a stringent secondary CFU-S assay at 24 weeks), a number that would be difficult to achieve in a clinical setting. The BERK→BERK transplantation model, however, shows that 20% autologous HSC correction may suffice for a significant amelioration of sickling, organ damage, and survival. However, whether this percentage of gene-modified HSCs necessary for effective gene therapy is achievable is critical to determine, since there is no survival advantage to the gene-modified HSCs in this disease. High human HSC transduction has been a limitation of gene therapy with the traditional gammaretrovirus vectors. Lentivirus vectors can overcome this barrier: a 20% long-term transduction has been shown in adrenoleukodystrophy with a lentivirus vector. Lentivirus transduction into human CD34+ cells was optimized, using the SCID-repopulating cell assay and achieved approximately 75% gene transfer in SCID-repopulating cell, on average, similar to previous data in human thalassemia CD34+ cells, where 70% transduction was seen 3 to 4 months after transplantation into immune-deficient mice. Notably, this level of gene transfer in the SCID mice is encouraging, and indeed higher than the gene transfer observed in NOD-SCID mice with the adrenoleukodystrophy lentivirus vector in preclinical studies.

Gene therapy using this approach could also overcome the toxicity and immunologic consequences of the traditional allogeneic bone marrow transplantation/reduced-intensity transplantation. Mismatched mixed chimerism of normal and sickle marrow in murine transplantations shows that a near complete chimerism is typically necessary for correction of organ damage. It is encouraging that, in a clinical series, reduced-intensity conditioning (RIC) transplantation with 8 mg/kg busulfan along with fludarabine, antithymocyte globulin, and total lymphoid irradiation in SCA patients has shown an average allogeneic engraftment of 78% at 2 to 8.5 years after transplantation, with correction of SCA phenotype. This high level of donor chimerism even in an allogeneic RIC setting, where immune rejection can occur, suggests that high gene transfer efficiency into autologous CD34+ cells followed by RIC may be a potentially safer alternative to myeloablative conditioning. 77% gene transfer efficiency in human stem/progenitors was demonstrated using a NOD-SCID repopulating cell assay, as well a correction of phenotype in mice with 1.3 to 1.5 copies per cell and approximately 20% gene-marked CFU-Ss (FIG. 36).

Significantly, correction occurred at 1 to 3 vector copies per cell, a clinically achievable goal. Flanking the GbG virus with a chromatin insulator is expected to increase HbF/vector copy by 2- to 4-fold. In experimental models, the insulator appears to reduce clonal dominance, although whether the insulator element lowers the risk of insertional oncogenesis is unknown. The risk of insertional oncogenesis observed with randomly integrating vectors has been shown to be lower with a lentivirus vector than a gammaretrovirus vector. It would be further lowered when the enhancer element is active only in a restricted erythroid lineage.

### Example 98

### Optimizing Closed-System Production of High-Titer Retroviral Vectors

The need for clinical grade gamma-retroviral vectors with self-inactivating (SIN) long terminal repeats has prompted a shift in the method with which large scale cGMP-grade vectors are produced, from the use of stable producer lines to transient transfection-based techniques. The Vector Production Facility, an academic cGMP manufacturing laboratory that is part of the Translational Core Laboratories at the Cincinnati Children's Research Foundation, has developed such a method based on the Wave Bioreactor® (GE Healthcare) production platform. This platform allows for large scale closed-system production of high-titer retroviral vectors for clinical trials using transient transfection up to 25 Liters per harvest using closed system processing.

The present study describes the development and scale-up procedures and reports on the successful use of the Wave Bioreactor in the production of six cGMP grade retroviral vectors in support of the FDA's National Toxicology Program (NTP).

### Example 99

### Transfection

Adherent 293T cells were transfected in T75 or T225 flasks or on 2 gram of FibraCel discs in ridged 850 cm² roller bottles (10 mL/T75; 30 mL/T225; 100 mL/roller bottle). Non-adherent 293F cells were grown in suspension culture and transfected in either serum-free FreeStyle 293 media (non-adherent conditions), or in FreeStyle media or DMEM supplemented with FBS (adherent conditions) in tissue culture flasks. Transfections were done using Calcium Phosphate (adherent conditions only), Lipofectamine 2000, or Fecturin according to the manufacturer's instructions. Vector was collected at 12 or 24 hour intervals, filtered at 0.45 μm, and frozen at or below −70° C. In the Bioreactor (suspension cells or adherent cells on Fibracel), higher titers were obtained when a higher concentration of plasmid was utilized (9.2 performed better than 6.9 or 4.6 microgram of total plasmid/mL media). Higher concentrations were not tested but may result in even further enhancements.

### Example 100

### Large Scale Virus Production

Cells from a certified 293T master cell bank (MCB) were expanded on tissue culture plastic, harvested, mixed with calcium phosphate transfection reagents and plasmid (4 g vector, 3.6 gram gag/pol, 1.6 gram env per Liter), and pumped into a Wave Cell Bag (GE Healthcare) containing FibraCel® discs (New Brunswick) in DMEM with 10% FBS (D10). Cells were cultured at 37° C., 5% CO2 using a rocking speed of 22 rpm and 6° angle. At 16-20 hours post-transfection, the media was exchanged; virus was harvested at approximately 12-hour intervals, filtered through a leukocyte reduction filter (Pall), aliquoted into Cryocyte freezing containers using a closed system fluid path, placed in protective freezing cassettes and frozen at or below −70° C.

### Example 101

### Titration

Vector pseudotyped with an ecotropic envelope was titered on NIH 3T3 cells, vector pseudotyped with the Gibbon Ape Leukemia (GALV) or Feline Leukemia Virus (RD114) envelope was titered on HT1080 cells. Titers were calculated based on the % GFP expression as determined by FACS or based on copy number as determined by vector specific quantitative PCR.

### Example 102

### Suspension Culture

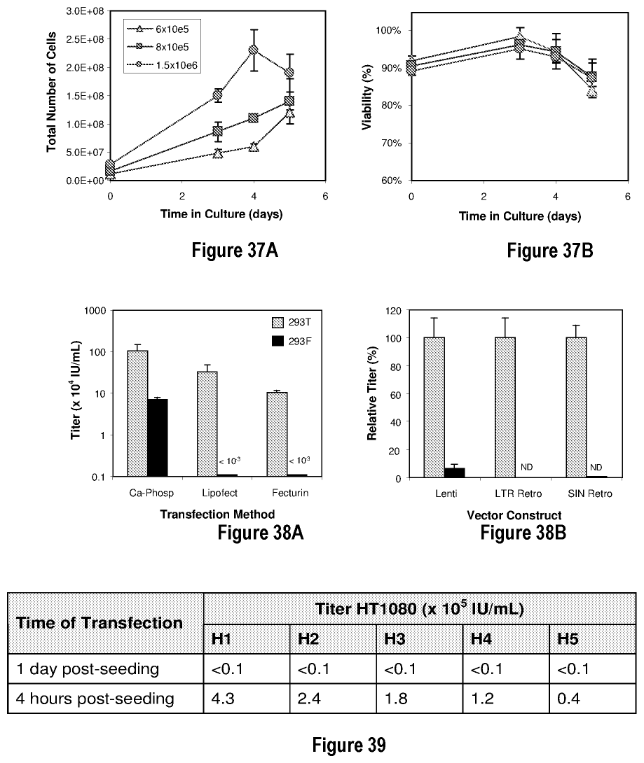

Initial pilot studies and scale-up were done with HEK293-derived 293F cells (Invitrogen) grown in serum-free (SF) FreeStyle 293 media (Invitrogen) as suspension cells are easier to manipulate in a bioreactor. Studies show up to 10-fold expansion over 5 days with cell viability at or above 80% (FIG. 37). However, 293F cells produced a 20-fold lower titer when transfected under adherent conditions in D10 with Ca-Phosphate (FIG. 38) and no detectable titer with other transfection reagents or under non-adherent conditions.

### Example 103

### Adherent Cell Culture

FibraCel disks (New Brunswick Scientific) are available as a sterile pre-loaded substrate for the Wave Bioreactor (at 20 gram per Liter) to support growth of adherent cells. Small scale pilot studies using adherent 293T cells were performed in 850 cm² ridged roller bottles with 2 gram FibraCel discs per 2×10⁸ 293T cells per 100 mL of D10. Post-seeding, cells migrate inside of the matrix and continue to expand as can be determined by glucose consumption over time. Glucose levels in a 1 Liter bioreactor that had been seeded with 2×10⁹ transfected 293T cells showed that the media should be changed at approximately 12 hour intervals to maintain a glucose level above 100 mg/dL. Treatment with TrypLESelect for up to 30 minutes allows up to 20% of the post-production cells to be released and harvested while the majority of cells maintain trapped in the matrix.

### Example 104

### Time of Transfection

To determine the optimal time of transfection, 293T cells were seeded onto FibraCel and exposed to transfection reagents and plasmid DNA within hours of seeding as compared to cells that were transfected the following day. The data show a titer of less than 104 IU/mL from cells that were transfected one day post-seeding as compared to cells that were transfected the same day (FIG. 39). It has now been determined that optimal titers are achieved when cells are mixed with transfection reagents and plasmid DNA at the time of seeding onto FibraCel.

### Example 105

### Plasmid DNA

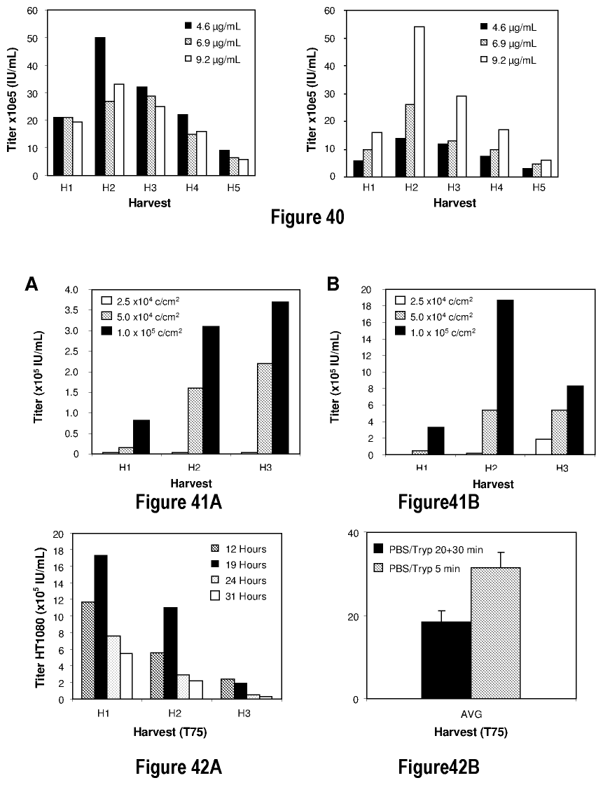

To establish the amount of plasmid DNA needed for optimal titer, 293T cells were transfected side-by-side on tissue culture plastic as well as on FibraCel. Where increasing plasmid DNA in static cultures produced a lower titer, increasing the DNA concentration on FibraCel increased titer as shown in a representative dataset (FIG. 40) out of a total of 3 experiments.

### Example 106

### Cell Culture

Cells were plated at different cell densities (from 2.5×10⁴ cells/cm² through 1×10⁵ cells/cm²) 4 days prior to transfection, harvested and tested for virus production in five separate experiments using GALV pseudotyped gamma-retroviral vectors. Although the same number of cells was used for each group, titers on plastic surface as well as on Fibracel cultures in the bioreactor varied greatly based on the plating density and were higher when cells were harvested from plates that had been seeded with a higher cell density (>2.5×10⁴ cells/cm²) (FIG. 41).

### Example 107

### Scale-Up

Several parameters were tested including the time of media change post-transfection (FIG. 42A) and the length of time cells were exposed to PBS and TrypLESelect prior to transfection (FIG. 42B). For media change, 19 hours was found to be optimal in two separate experiments (representative experiment shown). Although all cells had >95% viability after exposure to PBS and TrypLESelect, cells exposed for a shorter period of time generated higher titers.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the specific number of antigens in a screening panel or targeted by a therapeutic product, the type of antigen, the type of cancer, and the particular antigen(s) specified. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

Description of the 3′ Region of cHS4 Insulator can be found in, for example, Felsenfeld et al. (2004) Chromatin boundaries and chromatin domains. Cold Spring Harb Symp Quant Biol 69: 245-250; Neff T, et al. (1997) Stem cell gene therapy, position effects and chromatin insulators. Stem Cells 15: Suppl 1265-271; Challita and Kohn (1994) Lack of expression from a retroviral vector after transduction of murine hematopoietic stem cells is associated with methylation in vivo. Proc Natl Acad Sci USA 91 (7): 2567-71; Osborne C S, et al. (1999) Amelioration of retroviral vector silencing in locus control region beta-globin-transgenic mice and transduced F9 embryonic cells. J Virol 73: 5490-5496; Aker M, et al. (2007) Extended core sequences from the cHS4 insulator are necessary for protecting retroviral vectors from silencing position effects. Hum Gene Ther 18: 333-343; Kalberer C P, et al. (2000) Preselection of retrovirally transduced bone marrow avoids subsequent stem cell gene silencing and age-dependent extinction of expression of human beta-globin in engrafted mice. Proc Natl Acad Sci USA 97: 5411-5415; Hacein-Bey-Abina S, et al. (2003) LMO2-associated clonal T cell proliferation in two patients after gene therapy for SCID-X 1. Science 302: 415-419. Ott M G; et al. (2006) Correction of X-linked chronic granulomatous disease by gene therapy, augmented by insertional activation of MDS1-EVI1, PRDM16 or SETBP1. Nat Med 12: 401-409; Cavazzana-Calvo M, et al. (2000) Gene therapy of human severe combined immunodeficiency (SCID)-X1 disease. Science 288: 669-672; Zufferey R, et al. (1998) Self-inactivating lentivirus vector for safe and efficient in vivo gene delivery. J Virol 72: 9873-9880; Miyoshi H, et al. (1998) Development of a self-inactivating lentivirus vector. J Virol 72: 8150-8157; Kraunus J, et al. (2004) Self-inactivating retroviral vectors with improved RNA processing. Gene Ther 11: 1568-1578; Modlich U, et al. (2006) Cell-culture assays reveal the importance of retroviral vector design for insertional genotoxicity. Blood 108: 2545-2553; Zychlinski D, et al. (2008) Physiological promoters reduce the genotoxic risk of integrating gene vectors. Mol Ther 16: 718-725; Evans-Galea M V, et al. (2007) Suppression of clonal dominance in cultured human lymphoid cells by addition of the cHS4 insulator to a lentiviral vector. Mol Ther 15: 801-809; Ryu B Y, et al. (2007) A chromatin insulator blocks interactions between globin regulatory elements and cellular promoters in erythroid cells. Blood Cells Mol Dis 39: 221-228; Arumugam P I, et al. (2007) Improved human beta-globin expression from self-inactivating lentiviral vectors carrying the chicken hypersensitive site-4 (cHS4) insulator element. Mol Ther 15: 1863-1871; Emery D W, et al. (2002) Development of virus vectors for gene therapy of beta chain hemoglobinopathies: flanking with a chromatin insulator reduces gamma-globin gene silencing in vivo. Blood 100: 2012-2019; Imren S, et al. (2002) Permanent and panerythroid correction of murine beta thalassemia by multiple lentiviral integration in hematopoietic stem cells. Proc Natl Acad Sci USA 99: 14380-14385; Puthenveetil G, et al. (2004) Successful correction of the human beta-thalassemia major phenotype using a lentiviral vector. Blood 104: 3445-3453; Arumugam P I, et al. (2007) Improved human beta-globin expression from self-inactivating lentiviral vectors carrying the chicken hypersensitive site-4 (cHS4) insulator element. Mol Ther 15 (10): 1863-71; Rivella S, et al. (2000) The cHS4 insulator increases the probability of retroviral expression at random chromosomal integration sites. J Virol 74: 4679-4687; Ryu B Y, et al. (2008) An experimental system for the evaluation of retroviral vector design to diminish the risk for proto-oncogene activation. Blood 111: 1866-1875; Urbinati F, et al. (2009) Mechanism of Reduction in Titers From Lentivirus Vectors Carrying Large Inserts in the 3′ LTR. Mol Ther [Epub ahead of print]; Chung J H, et al. (1997) Characterization of the chicken beta-globin insulator. Proc Natl Acad Sci USA 94: 575-580; Recillas-Targa F, et al. (2002) Position-effect protection and enhancer blocking by the chicken beta-globin insulator are separable activities. Proc Natl Acad Sci USA 99: 6883-6888; Yusufzai T M, Felsenfeld G (2004) The 5′-HS4 chicken beta-globin insulator is a CTCF-dependent nuclear matrix-associated element. Proc Natl Acad Sci USA 101: 8620-8624; West A G, et al. (2004) Recruitment of histone modifications by USF proteins at a vertebrate barrier element. Mol Cell 16: 453-463. Huang S, et al. (2007) USF1 recruits histone modification complexes and is critical for maintenance of a chromatin barrier. Mol Cell Biol 27: 7991-8002; Parelho V, et al. (2008) Cohesins functionally associate with CTCF on mammalian chromosome arms. Cell 132: 422-433; Xie X, et al. (2007) Systematic discovery of regulatory motifs in conserved regions of the human genome, including thousands of CTCF insulator sites. Proc Natl Acad Sci USA 104: 7145-7150; Yusufzai T M, et al. (2004) CTCF tethers an insulator to subnuclear sites, suggesting shared insulator mechanisms across species. Mol Cell 13: 291-298; Smith S T, et al. (2009) Genome wide ChIP-chip analyses reveal important roles for CTCF in Drosophila genome organization. Dev Biol 328: 518-528; Cuddapah S, et al. (2009) Global analysis of the insulator binding protein CTCF in chromatin barrier regions reveals demarcation of active and repressive domains. Genome Res 19: 24-32; Kim T H, et al. (2007) Analysis of the vertebrate insulator protein CTCF-binding sites in the human genome. Cell 128: 1231-1245; Moreau-Gaudry F, et al. (2001) High-level erythroid-specific gene expression in primary human and murine hematopoietic cells with self-inactivating lentiviral vectors. Blood 98: 2664-2672; Perumbeti A, et al. (2009) A novel human gamma-globin gene vector for genetic correction of sickle cell anemia in a humanized sickle mouse model: critical determinants for successful correction. Blood [Epub ahead of print]; Puthenveetil G, et al. (2004) Successful correction of the human beta-thalassemia major phenotype using a lentiviral vector. Blood 104: 3445-3453; Shang Y, et al. (2000) Cofactor dynamics and sufficiency in estrogen receptor-regulated transcription. Cell 103: 843-852; Mutskov V J, et al. (2002) The barrier function of an insulator couples high histone acetylation levels with specific protection of promoter DNA from methylation. Genes Dev 16: 1540-1554; Modlich U, et al. (2006) Cell-culture assays reveal the importance of retroviral vector design for insertional genotoxicity. Blood 108: 2545-2553; Carr J M, et al. (2001) Effect of deletion and the site of insertion in double copy anti-tat retroviral vectors: viral titers and production of anti-tat mRNA. Arch Virol 146: 2191-2200; Junker U, et al. (1995) Genetic instability of a MoMLV-based antisense double-copy retroviral vector designed for HIV-1 gene therapy. Gene Ther 2: 639-646; Pathak V K, et al. (1990) Broad spectrum of in vivo forward mutations, hypermutations, and mutational hotspots in a retroviral shuttle vector after a single replication cycle: deletions and deletions with insertions. Proc Natl Acad Sci USA 87: 6024-6028; Zhuang J, et al. (2002) Human immunodeficiency virus type 1 recombination: rate, fidelity, and putative hot spots. J Virol 76: 11273-11282; Mohamedali A, et al. (2004) Self-inactivating lentiviral vectors resist proviral methylation but do not confer position-independent expression in hematopoietic stem cells. Mol Ther 10: 249-259; May C, et al. (2000) Therapeutic haemoglobin synthesis in beta-thalassaemic mice expressing lentivirus-encoded human beta-globin. Nature 406: 82-86; Bell A C, et al. (1999) The protein CTCF is required for the enhancer blocking activity of vertebrate insulators. Cell 98:387-396; Yannaki E, et al. (2002) Topological constraints governing the use of the chicken HS4 chromatin insulator in oncoretrovirus vectors. Mol Ther 5: 589-598; Saitoh N, et al. (2000) Structural and functional conservation at the boundaries of the chicken beta-globin domain. EMBO J 19: 2315-2322; Stumph W E, et al. (1984) Genomic structure and possible retroviral origin of the chicken CRI repetitive DNA sequence family. Proc Natl Acad Sci USA 81: 6667-6671; Gerasimova T I, et al. (2000) A chromatin insulator determines the nuclear localization of DNA. Mol Cell 6: 1025-1035; Pant V, et al. (2004) Mutation of a single CTCF target site within the H19 imprinting control region leads to loss of Igf2 imprinting and complex patterns of de novo methylation upon maternal inheritance. Mol Cell Biol 24: 3497-3504; Kim A, et al. (2007) Distinctive signatures of histone methylation in transcribed coding and noncoding human beta-globin sequences. Mol Cell Biol 27: 1271-1279; Litt M D, et al. (2001) Correlation between histone lysine methylation and developmental changes at the chicken beta-globin locus. Science 293: 2453-2455; Litt M D, et al. (2001) Transitions in histone acetylation reveal boundaries of three separately regulated neighboring loci. EMBO J 20:2224-2235; Li C L, et al. (2008) The cHS4 chromatin insulator reduces gammaretroviral vector silencing by epigenetic modifications of integrated provirus. Gene Ther 15: 49-53; Pikaart M J, et al. (1998) Loss of transcriptional activity of a transgene is accompanied by DNA methylation and histone deacetylation and is prevented by insulators. Genes Dev 12: 2852-2862; Bell A, et al. (1998) The establishment of active chromatin domains. Cold Spring Harb Symp Quant Biol 63: 509-514; Bell A C, et al. (2001) Insulators and boundaries: versatile regulatory elements in the eukaryotic. Science 291: 447-450; Yao S, et al. (2003) Retrovirus silencer blocking by the cHS4 insulator is CTCF independent. Nucleic Acids Res 31: 5317-5323; Elnitski L, et al. (1997) Conserved E boxes function as part of the enhancer in hypersensitive site 2 of the beta-globin locus control region. Role of basic helix-loop-helix proteins. J Biol Chem 272: 369-378; Crusselle-Davis V J, et al. (2006) Antagonistic regulation of beta-globin gene expression by helix-loop-helix proteins USF and TFII-I. Mol Cell Biol 26: 6832-6843; Pannell D, et al. (2000) Retrovirus vector silencing is de novo methylase independent and marked by a repressive histone code. EMBO J 19:5884-5894; Splinter E, et al. (2006) CTCF mediates long-range chromatin looping and local histone modification in the beta-globin locus. Genes Dev 20: 2349-2354; Kurukuti S, et al. (2006) CTCF binding at the H19 imprinting control region mediates maternally inherited higher-order chromatin conformation to restrict enhancer access to Igf2. Proc Natl Acad Sci USA 103: 10684-10689; Wendt K S, et al. (2008) Cohesin mediates transcriptional insulation by CCCTC-binding factor. Nature 451: 796-801; Wallace J A, et al. (2007) We gather together: insulators and genome organization. Curr Opin Genet Dev 17:400-407; Williams A, et al. (2008) The role of CTCF in regulating nuclear organization. J Exp Med 205: 747-750; each of which is hereby incorporated by reference in its entirety.

Description of reprogramming erythroid cells for lysosomal enzyme production and the effects on visceral and CNS cross-correction in mice with Hurler syndrome can be found in, for example, Hopwood and Morris (1990) The mucopolysaccharidoses. Diagnosis, molecular genetics and treatment. Mol Biol Med 7: 381-4041; Souillet G, et al. (2003) Outcome of 27 patients with Hurler's syndrome transplanted from either related or unrelated haematopoietic stem cell sources. Bone Marrow Transplant 31: 1105-1117; Staba S L, et al. (2004) Cord-blood transplants from unrelated donors in patients with Hurler's syndrome. N Engl J Med 350: 1960-1969; Grewal S, et al. (2003) Continued neurocognitive development and prevention of cardiopulmonary complications after successful BMT for I-cell disease: a long-term follow-up report. Bone Marrow Transplant 32: 957-960; Peters C, et al. (1996) Outcome of unrelated donor bone marrow transplantation in 40 children with Hurler syndrome. Blood 87: 4894-4902; Pan D, et al. (2004) Improved gene transfer and normalized enzyme levels in primitive hematopoietic progenitors from patients with mucopolysaccharidosis type I using a bioreactor. J Gene Med 6: 1293-1303; Hacein-Bey-Abina S, et al. (2002) Sustained correction of X-linked severe combined immunodeficiency by ex vivo gene therapy. N Engl J Med 346: 1185-1193; Aiuti A, et al. (2002) Correction of ADA-SOD by stem cell gene therapy combined with nonmyeloablative conditioning. Science 296: 2410-2413; Gaspar H B, et al. (2006) Successful reconstitution of immunity in ADA-SCID by stem cell gene therapy following cessation of PEG-ADA and use of mild preconditioning. (Translated from eng) Mol Ther 14: 505-513; Ott M G, et al. (2006) Correction of X-linked chronic granulomatous disease by gene therapy, augmented by insertional activation of MDS1-EVI1, PRDM16 or SETBP1. Nat Med 12: 401-409; Hacein-Bey-Abina S, et al. (2003) LMO2-associated clonal T cell proliferation in two patients after gene therapy for SCID-X1. Science 302: 415-419; Howe S J, et al. (2008) Insertional mutagenesis combined with acquired somatic mutations causes leukemogenesis following gene therapy of SCID-X1 patients. (Translated from eng) J Clin Invest 118: 3143-3150; Hacein-Bey-Abina S, et al. (2008) Insertional oncogenesis in 4 patients after retrovirus-mediated gene therapy of SCID-X1. J Clin Invest 118: 3132-3142; Kohn D B, et al. (2003) Occurrence of leukaemia following gene therapy of X-linked SCID. Nature reviews 3: 477-488; von Kalle C, et al. (2004) Lenti in red: progress in gene therapy for human hemoglobinopathies. J Clin Invest 114: 889-891; Chang A H, et al. (2006) Stem cell-derived erythroid cells mediate long-term systemic protein delivery. Nat Biotechnol 24: 1017-1021; Moreau-Gaudry F, et al. (2001) High-level erythroid-specific gene expression in primary human and murine hematopoietic cells with self-inactivating lentiviral vectors. Blood 98: 2664-2672; Scott H S, et al. (1991) Human alpha-L-iduronidase: cDNA isolation and expression. Proc Natl Acad Sci USA 88: 9695-9699; Zhao K W, et al. (1997) Carbohydrate structures of recombinant human alpha-L-iduronidase secreted by Chinese hamster ovary cells. J Biol Chem 272: 22758-22765; Kalfa T A, et al. (2009) Rac 1 and Rac2 GTPases are necessary for early erythropoietic expansion in the bone marrow but not in the spleen. Haematologica; McGrath K E, et al. (2008) Multispectral imaging of hematopoietic cells: where flow meets morphology. J Immunol Methods 336: 91-97; Socolovsky M, et al. (2001) Ineffective erythropoiesis in Stat5a(−/−)5b(−/−) mice due to decreased survival of early erythroblasts. Blood 98:3261-3273; Pan D, et al. (2008) Progression of multiple behavioral deficits with various ages of onset in a murine model of Hurler syndrome. Brain Res 1188: 241-253; Brooks D A, et al. (2001) Glycosidase active site mutations in human alpha-L-iduronidase. Glycobiology 11: 741-750; Ney P A (2006) Gene expression during terminal erythroid differentiation. Curr Opin Hematol 13: 203-208; Baum C, et al. (2003) Side effects of retroviral gene transfer into hematopoietic stem cells. Blood 101: 2099-2114; Modlich U, et al. (2006) Cell-culture assays reveal the importance of retroviral vector design for insertional genotoxicity. Blood 108: 2545-2553; Bushman F D (2007) Retroviral integration and human gene therapy. J Clin Invest 117: 2083-2086; Metals J Y and Dunbar C E (2008) The MDS1-EVI1 gene complex as a retrovirus integration site: impact on behavior of hematopoietic cells and implications for gene therapy. Mol Ther 16: 439-449; Ellis J (2005) Silencing and variegation of gammaretrovirus and lentivirus vectors. Hum Gene Ther 16: 1241-1246; Chang A H and Sadelain M (2007) The genetic engineering of hematopoietic stem cells: the rise of lentiviral vectors, the conundrum of the ltr, and the promise of lineage-restricted vectors. Mol Ther 15: 445-456; Geminard C, et al. (2002) Reticulocyte maturation: mitoptosis and exosome release. Biocell 26: 205-215; Chasis J A and Mohandas N (2008) Erythroblastic islands: niches for erythropoiesis. Blood 112: 470-478; Sabatino D E, et al. (2000) A minimal ankyrin promoter linked to a human gamma-globin gene demonstrates erythroid specific copy number dependent expression with minimal position or enhancer dependence in transgenic mice. J Biol Chem 275: 28549-28554; Huang B L, et al. (1998) Derepression of human embryonic zeta-globin promoter by a locus-control region sequence. Proc Natl Acad Sci USA 95: 14669-14674; Surinya K H, et al. (1998) Identification and characterization of a conserved erythroid-specific enhancer located in intron 8 of the human 5-aminolevulinate synthase 2 gene. J Biol Chem 273: 16798-16809; Kennedy D W and Abkowitz J L (1998) Mature monocytic cells enter tissues and engraft. Proc Natl Acad Sci USA 95: 14944-14949; Biffi A, et al. (2006) Gene therapy of metachromatic leukodystrophy reverses neurological damage and deficits in mice. J Clin Invest 116: 3070-3082; Ohmi K, et al. (2003) Activated microglia in cortex of mouse models of mucopolysaccharidoses I and IIIB. Proc Natl Acad Sci USA 100: 1902-1907; Vogler C, et al. (2005) Overcoming the blood-brain barrier with high-dose enzyme replacement therapy in murine mucopolysaccharidosis VII. Proc Natl Acad Sci USA 102: 14777-14782; Roces D P, et al. (2004) Efficacy of enzyme replacement therapy in alpha-mannosidosis mice: a preclinical animal study. Hum Mol Genet 13: 1979-1988; Sly W S, et al. (2006) Enzyme therapy in mannose receptor-null mucopolysaccharidosis VII mice defines roles for the mannose 6-phosphate and mannose receptors. Proc Natl Acad Sci USA 103: 15172-15177; Grubb J H, et al. (2008) Chemically modified beta-glucuronidase crosses blood-brain barrier and clears neuronal storage in murine mucopolysaccharidosis VII. Proc Natl Acad Sci USA 105: 2616-2621; Worsham D N, et al. (2006) In vivo gene transfer into adult stem cells in unconditioned mice by in situ delivery of a lentiviral vector. Mol Ther 14: 514-524; Baum C, et al. (1998) cis-Active elements of Friend spleen focus-forming virus: from disease induction to disease prevention. Acta Haematol 99: 156-164; Pan D, et al. (2000) Retroviral vector design studies toward hematopoietic stem cell gene therapy for mucopolysaccharidosis type I. Gene Ther 7: 1875-1883; Pan D, et al. (2002) Biodistribution and toxicity studies of VSVG-pseudotyped lentiviral vector after intravenous administration in mice with the observation of in vivo transduction of bone marrow. Mol Ther 6: 19-29; Hartung S D, et al. (2004) Correction of metabolic, craniofacial, and neurologic abnormalities in MPS I mice treated at birth with adeno-associated virus vector transducing the human alpha-1-iduronidase gene. Mol Ther 9: 866-875; each of which is hereby incorporated by reference in its entirety.

Description of the Cis elements can be found for example, Aiuti A, et al. Immune reconstitution in ADA-SLID after PBL gene therapy and discontinuation of enzyme replacement. NatMed 2002; 8: 423-425; Cavazzana-Calvo M, et al. Gene therapy of human severe combined immunodeficiency (SCID)-X1 disease. Science 2000; 288: 669-672; Ott M G, et al. Correction of X-linked chronic granulomatous disease by gene therapy, augmented by insertional activation of MDS1-EVIL, PRDM16 or SETBP1. NatMed 2006; 12: 401-409; Cone R D, et al. Regulated expression of a complete human beta-globin gene encoded by a transmissible retrovirus vector. MolCell Biol 1987; 7: 887-897; Karlsson S, et al. Expression of the human beta-globin gene following retroviral-mediated transfer into multipotential hematopoietic progenitors of mice. Proc Natl Acad Sci USA 1988; 85: 6062-6066; Bender M A, et al. A majority of mice show long-term expression of a human beta-globin gene after retrovirus transfer into hematopoietic stem cells. Mol Cell Biol 1989; 9: 1426-1434; Dzierzak E, et al. Lineage-specific expression of a human β-globin gene in murine bone marrow transplant recipients reconstituted with retrovirus-transduced stem cells. Nature 1988; 331: 35-41; Buchman A R, Berg P. Comparison of intron-dependent and intron-independent gene expression. MolCell Biol 1988; 8: 4395-4405; Huang Y, et al. Intronless mRNA transport elements may affect multiple steps of pre-mRNA processing. EMBO J 1999; 18: 1642-1652; Leboulch P, et al. Mutagenesis of retroviral vectors transducing human beta-globin gene and beta-globin locus control region derivatives results in stable transmission of an active transcriptional structure. EMBO J 1994; 13: 3065-3076; Plavec I, et al. A human beta-globin gene fused to the human beta-globin locus control region is expressed at high levels in erythroid cells of mice engrafted with retrovirus-transduced hematopoietic stem cells. Blood 1993; 81: 1384-1392; Chang J C, et al. A 36-base-pair core sequence of locus control region enhances retrovirally transferred human beta-globin gene expression. Proc Natl Acad Sci USA 1992; 89: 3107-3110; Novak U, et al. High-level beta-globin expression after retroviral transfer of locus activation region-containing human beta-globin gene derivatives into murine erythroleukemia cells. Proc Natl Acad Sci 1990; 87: 3386-3390; Sadelain M, et al. Generation of a high-titer retroviral vector capable of expressing high levels of the human beta-globin gene. Proc Natl Acad Sci 1995; 92: 6728-6732; May C, et al. Therapeutic haemoglobin synthesis in beta-thalassaemic mice expressing lentivirus-encoded human beta-globin. Nature 2000; 406: 82-86; Imren S, et al. Permanent and panerythroid correction of murine beta thalassemia by multiple lentiviral integration in hematopoietic stem cells. Proc Natl Acad Sci 2002; 99: 14380-14385; Persons D A, et al. The degree of phenotypic correction of murine beta-thalassemia intermedia following lentiviral-mediated transfer of a human gamma-globin gene is influenced by chromosomal position effects and vector copy number. Blood 2003; 101: 2175-2183; Puthenveetil and Malik. Gene therapy for hemoglobinopathies: are we there yet? CurrHematolRep 2004; 3: 298-305; Schambach A, et al. Overcoming promoter competition in packaging cells improves production of self-inactivating retroviral vectors. Gene Ther Gene therapy 2006; 13: 1524-1533; Puthenveetil G, et al. Successful correction of the human beta-thalassemia major phenotype using a lentiviral vector. Blood 2004; 104: 3445-3453; Arumugam P I, et al. Improved Human beta-globin Expression from Self-inactivating Lentiviral Vectors Carrying the Chicken Hypersensitive Site-4 (cHS4) Insulator Element. Mol Ther 2007; Dull T, et al. A third-generation lentivirus vector with a conditional packaging system. J Virol 1998; 72: 8463-8471; Cui Y, et al. Contributions of viral splice sites and cis-regulatory elements to lentivirus vector function. J Virol 1999; 73:6171-6176; Richardson J H, et al. Packaging of human immunodeficiency virus type 1 RNA requires cis-acting sequences outside the 5′ leader region. J Virol Journal of virology 1993; 67: 3997-4005; Buchschacher G L, Jr., Panganiban A T. Human immunodeficiency virus vectors for inducible expression of foreign genes. J Virol Journal of virology 1992; 66: 2731-2739; Parolin C, et al. Analysis in human immunodeficiency virus type 1 vectors of cis-acting sequences that affect gene transfer into human lymphocytes. J Virol Journal of virology 1994; 68: 3888-3895; Baudin F, et al. Functional sites in the 5′ region of human immunodeficiency virus type 1 RNA form defined structural domains. J Mol Biol 1993; 229: 382-397; Luban J, Goff S P. Binding of human immunodeficiency virus type 1 (HIV-1) RNA to recombinant HIV-1 gag polyprotein. J Virol 1991; 65: 3203-3212; Sundquist W I, Heaphy S. Evidence for interstrand quadruplex formation in the dimerization of human immunodeficiency virus 1 genomic RNA. Proc Natl Acad Sci U SA 1993; 90: 3393-3397; Favaro J P, et al. Effect of Rev on the intranuclear localization of HIV-1 unspliced RNA. Virology 1998; 249: 286-296; Berthold E, Maldarelli F. cis-acting elements in human immunodeficiency virus type 1 RNAs direct viral transcripts to distinct intranuclear locations. J Virol 1996; 70: 4667-4682; Shin N H, et al. Replication of lengthened Moloney murine leukemia virus genomes is impaired at multiple stages. J Virol Journal of virology 2000; 74: 2694-2702; Hanenberg H, et al. Phenotypic correction of primary Fanconi anemia T cells with retroviral vectors as a diagnostic tool. Exp Hematol 2002; 30: 410-420; Kelly P F, et al. Stem cell collection and gene transfer in fanconi anemia. MolTher 2007; 15: 211-219; Kumar M, et al. Systematic determination of the packaging limit of lentiviral vectors. Hum Gene Ther Human gene therapy 2001; 12: 1893-1905; Kohn D B. Lentiviral vectors ready for prime-time. Nat Biotechnol Nature biotechnology 2007; 25: 65-66; Hildinger M, et al. Design of 5′ untranslated sequences in retroviral vectors developed for medical use. J Virol 1999; 73: 4083-4089; Yu S S, et al. Construction of a retroviral vector production system with the minimum possibility of a homologous recombination. Gene Ther 2003; 10: 706-711; Malim M H, et al. The HIV-1 rev trans-activator acts through a structured target sequence to activate nuclear export of unspliced viral mRNA. Nature Nature 1989; 338: 254-257; Chang D D, Sharp P A. Messenger RNA transport and HIV rev regulation. Science 1990; 249: 614-615; Fischer U, et al., Luhrmann R, Rautmann G. Evidence that HIV-1 Rev directly promotes the nuclear export of unspliced RNA. EMBO J 1994; 13: 4105-4112; Anson D S, Fuller M. Rational development of a HIV-1 gene therapy vector. J Gene Med 2003; 5: 829-838; Felber B K, et al. rev protein of human immunodeficiency virus type 1 affects the stability and transport of the viral mRNA. Proc Natl Acad Sci USA 1989; 86: 1495-1499; Brandt S, et al. Rev proteins of human and simian immunodeficiency virus enhance RNA encapsidation. PLoS Pathog 2007; 3: e54; Swanson C M, et al. Retroviral mRNA nuclear export elements regulate protein function and virion assembly. Embo J 2004; 23: 2632-2640; Kaye J F, et al. cis-acting sequences involved in human immunodeficiency virus type 1 RNA packaging. J Virol Journal of virology 1995; 69: 6588-6592; Schwartz S, et al. Mutational inactivation of an inhibitory sequence in human immunodeficiency virus type 1 results in Rev-independent gag expression. J Virol Journal of virology 1992; 66: 7176-7182; Moreau-Gaudry F, et al. High-level erythroid-specific gene expression in primary human and murine hematopoietic cells with self-inactivating lentiviral vectors. Blood 2001; 98: 2664-2672; Kraunus J, et al. Murine leukemia virus regulates alternative splicing through sequences upstream of the 5′ splice site. J Biol Chem 2006; 281: 37381-37390; each of which is hereby incorporated by reference in its entirety.

Description of the relationship between titer and the insert length into LTR can be found in, for example, Rivella, S, et al. (2003). A novel murine model of Cooley anemia and its rescue by lentiviral-mediated human beta-globin gene transfer. Blood 101: 2932-2939; Persons, D A, et al. (2003). The degree of phenotypic correction of murine beta-thalassemia intermedia following lentiviral-mediated transfer of a human gamma-globin gene is influenced by chromosomal position effects and vector copy number. Blood 101: 2175-2183; Modlich, U, et al. (2006). Cell-culture assays reveal the importance of retroviral vector design for insertional genotoxicity 108: 2545-2553; Imren, S, et al. (2002). Permanent and panerythroid correction of murine beta thalassemia by multiple lentiviral integration in hematopoietic stem cells 99: 14380-14385; Thrasher, A J, et al. (2006). Gene therapy: X-SCID transgene leukaemogenicity 443: E5-E6; Fischer, A, Abina, SH, Thrasher, A, von Kalle, C, and Cavazzana-Calvo, M (2004). LMO2 and gene therapy for severe combined immunodeficiency 350: 2526-2527; Hacein-Bey-Abina, S, et al. (2003). LMO2-associated clonal T cell proliferation in two patients after gene therapy for SCID-X 1. Science 302: 415-419; Zufferey, R, et al. (1998). Self-inactivating lentivirus vector for safe and efficient in vivo gene delivery 72: 9873-9880; Miyoshi, H, et al. (1998). Development of a self-inactivating lentivirus vector 72: 8150-8157; Kraunus, J, et al. (2004). Self-inactivating retroviral vectors with improved RNA processing 11: 1568-1578; Chung, J H, et al. (1997). Characterization of the chicken beta-globin insulator 94: 575-580; Bell, A C, and Felsenfeld, G (1999). Stopped at the border: boundaries and insulators 9: 191-198; Felsenfeld, G, et al. (2004). Chromatin boundaries and chromatin domains 69: 245-50: 245-250; Rivella, S, et al. (2000). The cHS4 insulator increases the probability of retroviral expression at random chromosomal integration sites 74: 4679-4687; Emery, D W, et al. (2002). Development of virus vectors for gene therapy of beta chain hemoglobinopathies: flanking with a chromatin insulator reduces gamma-globin gene silencing in vivo. Blood 100: 2012-2019; Yannaki, E, et al. (2002). Topological constraints governing the use of the chicken HS4 chromatin insulator in oncoretrovirus vectors 5: 589-598; Ramezani, A, et al. (2003). Performance- and safety-enhanced lentiviral vectors containing the human interferon-beta scaffold attachment region and the chicken beta-globin insulator. Blood 101: 4717-4724; Arumugam, P I, et al. (2007). Improved Human beta-globin Expression from Self-inactivating Lentiviral Vectors Carrying the Chicken Hypersensitive Site-4 (cHS4) Insulator Element. Mol Ther; Puthenveetil, G, et al. (2004). Successful correction of the human beta-thalassemia major phenotype using a lentiviral vector. Blood 104: 3445-3453; Evans-Galea, et al. (2006). Supression of clonal dominance in cultured human lymphoid cells by addition of 5′ cHS4 insulator to a lentiviral vector. Molecular Therapy 13: S405; Ryu, B Y, et al. (2007). A chromatin insulator blocks interactions between globin regulatory elements and cellular promoters in erythroid cells. Blood Cells Mol Dis 39: 221-228; Carr, J M, et al. (2001). Effect of deletion and the site of insertion in double copy anti-tat retroviral vectors: viral titres and production of anti-tat mRNA. Archives of virology 146: 2191-2200; Junker, U, et al. (1995). Genetic instability of a MoMLV-based antisense double-copy retroviral vector designed for HIV-1 gene therapy. Gene Ther 2: 639-646; Shin, N H, et al. (2000). Replication of lengthened Moloney murine leukemia virus genomes is impaired at multiple stages. J Virol 74: 2694-2702; Gelinas, C, and Temin, HM (1988). The v-rel oncogene encodes a cell-specific transcriptional activator of certain promoters. Oncogene 3:349-355; Coffin, J M (1990). Retroviridae and their replication. In: Fields, BN (ed). Feilds Virology, 2 ed. New York: Raven Press. pp 1437-1500; Kumar, M, et al. (2001). Systematic determination of the packaging limit of lentiviral vectors. Hum Gene Ther 12: 1893-1905; Recillas-Targa, F, et al. (1999). Positional enhancer-blocking activity of the chicken beta-globin insulator in transiently transfected cells 96: 14354-14359; Julias, J G, et al. (2001). Replication of phenotypically mixed human immunodeficiency virus type 1 virions containing catalytically active and catalytically inactive reverse transcriptase. J Virol 75: 6537-6546; Brule, F, et al. (2000). In vitro evidence for the interaction of tRNA (3) (Lys) with U3 during the first strand transfer of HIV-1 reverse transcription. Nucleic Acids Res 28: 634-640; Ohi, Y, and Clever, J L (2000). Sequences in the 5′ and 3′ R elements of human immunodeficiency virus type 1 critical for efficient reverse transcription. J Virol 74: 8324-8334; Topping, R, et al. (1998). Cis-acting elements required for strong stop acceptor template selection during Moloney murine leukemia virus reverse transcription. J Mol Biol 281: 1-15; Pathak, V K, and Temin, HM (1990). Broad spectrum of in vivo forward mutations, hypermutations, and mutational hotspots in a retroviral shuttle vector after a single replication cycle: deletions and deletions with insertions. Proc Natl Acad Sci USA 87:6024-6028; Zhuang, J, et al. (2002). Human immunodeficiency virus type 1 recombination: rate, fidelity, and putative hot spots. J Virol 76: 11273-11282; Herman, S A, and Coffin, J M (1987). Efficient packaging of readthrough RNA in ALV: implications for oncogene transduction. Science 236: 845-848; Schambach, A, et al. (2007). Improving transcriptional termination of self-inactivating gamma-retroviral and lentiviral vectors. Mol Ther 15:1167-1173; Higashimoto, T, et al. (2007). The woodchuck hepatitis virus post-transcriptional regulatory element reduces readthrough transcription from retroviral vectors. Gene Ther. Zaiss, A K, et al. (2002). RNA 3′ readthrough of oncoretrovirus and lentivirus: implications for vector safety and efficacy 76:7209-7219; Heilman-Miller, S L, et al. (2004). Alteration of nucleic acid structure and stability modulates the efficiency of minus-strand transfer mediated by the HIV-1 nucleocapsid protein. J Biol Chem 279: 44154-44165; Negroni, M, and Buc, H (2000). Copy-choice recombination by reverse transcriptases: reshuffling of genetic markers mediated by RNA chaperones. Proc Natl Acad Sci USA 97: 6385-6390; Wu, T, et al. (2007). Effects of nucleic acid local structure and magnesium ions on minus-strand transfer mediated by the nucleic acid chaperone activity of HIV-1 nucleocapsid protein. Nucleic Acids Res 35: 3974-3987; Berkhout, B, et al. (2001). Structural features in the HIV-1 repeat region facilitate strand transfer during reverse transcription. RNA 7:1097-1114; Butler, S L, et al. (2001). A quantitative assay for HIV DNA integration in vivo. Nat Med 7: 631-634; Brown, P O, et al. (1987). Correct integration of retroviral DNA in vitro. Cell 49: 347-356; Bukrinsky, M I, et al. (1992). Active nuclear import of human immunodeficiency virus type 1 preintegration complexes. Proc Natl Acad Sci USA 89: 6580-6584; Zennou, V, et al. (2000). HIV-1 genome nuclear import is mediated by a central DNA flap. Cell 101: 173-185; Hansen, M S, et al. (1999). Integration complexes derived from HIV vectors for rapid assays in vitro. Nat Biotechnol 17: 578-582; Miller, M D, et al. (1997). Human immunodeficiency virus type 1 preintegration complexes: studies of organization and composition. J Virol 71: 5382-5390; Grande, A, et al. (1999). Transcriptional targeting of retroviral vectors to the erythroblastic progeny of transduced hematopoietic stem cells. Blood 93: 3276-3285; Wiznerowicz, M, et al. (1998). Development of a double-copy bicistronic retroviral vector for human gene therapy. Advances in experimental medicine and biology 451: 441-447; Wiznerowicz, M, et al. (1997). Double-copy bicistronic retroviral vector platform for gene therapy and tissue engineering: application to melanoma vaccine development. Gene Ther 4: 1061-1068; Junker, U, et al. (1997). Antiviral potency of drug-gene therapy combinations against human immunodeficiency virus type 1. AIDS research and human retroviruses 13: 1395-1402; Ilves, H, et al. (1996). Retroviral vectors designed for targeted expression of RNA polymerase III-driven transcripts: a comparative study. Gene 171: 203-208; Sabatino, D E, et al. (2000). A Minimal Ankyrin Promoter Linked to a Human gamma-Globin Gene Demonstrates Erythroid Specific Copy Number Dependent Expression with Minimal Position or Enhancer Dependence in Transgenic Mice 15: 28549-28554; Sabatino, D E, et al. (2000). Long-term expression of gamma-globin mRNA in mouse erythrocytes from retrovirus vectors containing the human gamma-globin gene fused to the ankyrin-1 promoter 97: 13294-13299; Moreau-Gaudry, F, et al. (2001). High-level erythroid-specific gene expression in primary human and murine hematopoietic cells with self-inactivating lentiviral vectors. Blood 98: 2664-2672; Yanez-Munoz, R J, et al. (2006). Effective gene therapy with nonintegrating lentiviral vectors. Nature medicine 12: 348-353; each of which is hereby incorporated by reference in its entirety.

Description of the Correction of Sickle Cell Anemia can be found for example, Darbari D S, et al. Circumstances of death in adult sickle cell disease patients. Am J Hematol. 2006; 81 (11): 858-863; Platt O S, et al. Mortality in sickle cell disease: life expectancy and risk factors for early death. N Engl J Med. 1994; 330 (23): 1639-1644; Davis H, et al. National trends in the mortality of children with sickle cell disease, 1968 through 1992. Am J Public Health. 1997; 87 (8): 1317-1322; World Health Organization. Sickle-Cell Anaemia Fifty-Ninth World Health Assembly, Provisional Agenda Item 11.4. 2006; A59: 1; Walters M C, et al. Barriers to bone marrow transplantation for sickle cell anemia. Biol Blood Marrow Transplant. 1996; 2 (2): 100-104; Fitzhugh C D, et al., Late effects of myeloablative bone marrow transplantation (BMT) in sickle cell disease (SCD) [letter]. Blood. 2008; 111 (3): 1742-1743; author reply 1744; Bernaudin F, et al. Long-term results of related myeloablative stem-cell transplantation to cure sickle cell disease. Blood. 2007; 110 (7): 2749-2756; Pawliuk R, et al.Correction of sickle cell disease in transgenic mouse models by gene therapy. Science. 2001; 294 (5550): 2368-2371; Levasseur D N, et al. Correction of a mouse model of sickle cell disease: lentiviral/antisickling beta-globin gene transduction of unmobilized, purified hematopoietic stem cells. Blood. 2003; 102 (13): 4312-4319; Puthenveetil G, et al. Successful correction of the human beta thalassemia major phenotype using a lentiviral vector. Blood. 2004; 104 (12): 3445-3453; Moreau-Gaudry F, et al. High-level erythroid-specific gene expression in primary human and murine hematopoietic cells with selfinactivating lentiviral vectors. Blood. 2001; 98 (9): 2664-2672; Rubin J E, et al. Locus control region activity by 5HS3 requires a functional interaction with beta-globin gene regulatory elements: expression of novel beta/gamma-globin hybrid transgenes. Blood. 2000; 95 (10): 3242-3249; Arumugam P I, et al. Improved human beta-globin expression from self-inactivating lentiviral vectors carrying the chicken hypersensitive site-4 (cHS4) insulator element. Mol Ther. 2007; 15 (10): 1863-1871; Pa′szty C, et al. Transgenic knockout mice with exclusively human sickle hemoglobin and sickle cell disease. Science. 1997; 278 (5339): 876-878; Manci E A, et al. Pathology of Berkeley sickle cell mice: similarities and differences with human sickle cell disease. Blood. 2006; 107 (4): 1651-1658; Baskurt O K, et al. Blood rheology and hemodynamics. Semin Thromb Hemost. 2003; 29 (5): 435-450; Iannone R, et al. Effects of mixed hematopoietic chimerism in a mouse model of bone marrow transplantation for sickle cell anemia. Blood. 2001; 97 (12): 3960-3965; Iannone R, et al. Results of minimally toxic nonmyeloablative transplantation in patients with sickle cell anemia and beta thalassemia. Biol Blood Marrow Transplant. 2003; 9 (8): 519-528; Hardeman M R, Ince C. Clinical potential of in vitro measured red cell deformability, a myth? Clin Hemorheol Microcirc. 1999; 21 (3-4): 277-284; Franco R S, et al. Time-dependent changes in the density and hemoglobin F content of biotin-labeled sickle cells. J Clin Invest. 1998; 101 (12): 2730-2740; Pestina T I, et al. Correction of murine sickle cell disease using gamma-globin lentiviral vectors to mediate high-level expression of fetal hemoglobin. Mol Ther. 2009; 17 (2): 245-252; Kohn D B, et al. T lymphocytes with a normal ADA gene accumulate after transplantation of transduced autologous umbilical cord blood CD34 cells in ADA-deficient SOD neonates. Nat Med. 1998; 4 (7): 775-780; Aiuti A, et al. Correction of ADASCID by stem cell gene therapy combined with nonmyeloablative conditioning. Science. 2002; 296 (5577): 2410-2413; Aiuti A, et al. Gene therapy for immunodeficiency due to adenosine deaminase deficiency. N Engl J Med. 2009; 360 (5): 447-458; Noronha P A, et al., Hemoglobin-specific antibody in a multiply transfused patient with sickle cell disease. Blood. 1997; 89 (6): 2155-2158; Samakoglu S, et al. A genetic strategy to treat sickle cell anemia by coregulating globin transgene expression and RNA interference. Nat Biotechnol. 2006; 24 (1): 89-94; Trudel M, et al. Sickle cell disease of transgenic SAD mice. Blood. 1994; 84 (9): 3189-3197; Blouin M J, et al. Genetic correction of sickle cell disease: insights using transgenic mouse models. Nat Med. 2000; 6 (2): 177-182; Fabry M E, et al. Second generation knockout sickle mice: the effect of HbF. Blood. 2001; 97 (2): 410-418; Persons D A, et al. The degree of phenotypic correction of murine beta-thalassemia intermedia following lentiviral-mediated transfer of a human gamma-globin gene is influenced by chromosomal position effects and vector copy number. Blood. 2003; 101 (6): 2175-2183; Koshy M, et al. 2-deoxy 5-azacytidine and fetal hemoglobin induction in sickle cell anemia. Blood. 2000; 96 (7): 2379-2384; Charache S, et al. Hydroxyurea-induced augmentation of fetal hemoglobin production in patients with sickle cell anemia. Blood. 1987; 69 (1): 109-116; Maier-Redelsperger M, et al. Fetal hemoglobin and F-cell responses to long-term hydroxyurea treatment in young sickle cell patients: The French Study Group on Sickle Cell Disease. Blood. 1998; 91 (12): 4472-4479; Marcus S J, Ware R E. Physiologic decline in fetal hemoglobin parameters in infants with sickle cell disease: implications for pharmacological intervention. J Pediatr Hematol Oncol. 1999; 21 (5): 407-411; Maier-Redelsperger M, et al. Variation in fetal hemoglobin parameters and predicted hemoglobin S polymerization in sickle cell children in the first two years of life: Parisian Prospective Study on Sickle Cell Disease. Blood. 1994; 84 (9): 3182-3188; Steinberg M H, et al. Effect of hydroxyurea on mortality and morbidity in adult sickle cell anemia: risks and benefits up to 9 years of treatment. JAMA. 2003; 289 (13): 1645-1651; Kean L S, et al. Chimerism and cure: hematologic and pathologic correction of murine sickle cell disease. Blood. 2003; 102 (13): 4582-4593; Walters M C, et al. Stable mixed hematopoietic chimerism after bone marrow transplantation for sickle cell anemia. Biol Blood Marrow Transplant. 2001; 7 (12): 665-673; Cavazzana-Calvo M, et al. Hematopoietic stem cell gene therapy trial with lentiviral vector in X-linked adrenoleukodystrophy. Blood (ASH Annual Meeting Abstracts). 2008; (suppl) 112. Abstract 821; Benhamida S, et al. Transduced CD34_cells from adrenoleukodystrophy patients with HIV-derived vector mediate long-term engraftment of NOD/SOD mice. Mol Ther. 2003; 7 (3): 317-324; Horan J T, et al. Hematopoietic stem cell transplantation for multiply transfused patients with sickle cell disease and thalassemia after low-dose total body irradiation, fludarabine, and rabbit antithymocyte globulin. Bone Marrow Transplant. 2005; 35 (2): 171-177; Krishnamurti L, et al. Bone marrow transplantation without myeloablation for sickle cell disease. N Engl J Med. 2001; 344 (1): 68; Kean L S, et al. A cure for murine sickle cell disease through stable mixed chimerism and tolerance induction after nonmyeloablative conditioning and major histocompatibility complex-mismatched bone marrow transplantation. Blood. 2002; 99 (5): 1840-1849; Krishnamurti L, et al. Stable long-term donor engraftment following reduced-intensity hematopoietic cell transplantation for sickle cell disease. Biol Blood Marrow Transplant. 2008; 14 (11): 1270-1278; Ryu B Y, et al. An experimental system for the evaluation of retroviral vector design to diminish the risk for proto-oncogene activation. Blood. 2008; 111 (4): 1866-1875; Ryu B Y, et al. A chromatin insulator blocks interactions between globin regulatory elements and cellular promoters in erythroid cells. Blood Cells Mol Dis. 2007; 39 (3): 221-228; Montini E, et al. Hematopoietic stem cell gene transfer in a tumor-prone mouse model uncovers low genotoxicity of lentiviral vector integration. Nat Biotechnol. 2006; 24 (6): 687-696; von Kalle C, et al. Lenti in red: progress in gene therapy for human hemoglobinopathies; each of which is hereby incorporated by reference in its entirety.

1-26. (canceled)

27. A modified self-inactivating (SIN) lentiviral vector, comprising:
(i) a packaging signal,
(ii) a gag gene fragment consisting of about 360 base pairs of the 5′ end of a gag gene,
(iii) a Rev response element (RRE),
(iv) an envelope fragment comprising a splice acceptor sequence (SA),
(v) a Central Polypurine Tract; and
(vi) a nucleic acid sequence encoding beta-globin and/or a nucleic acid sequence encoding gamma-globin,
wherein said lentiviral vector is capable of expressing beta globin and/or gamma globin.

28. The modified SIN lentiviral vector of claim 27, wherein the lentiviral vector further comprises a long terminal repeat (LTR) comprising a polyadenylation (polyA) signal sequence, said LTR being located at the 3′ end of said lentiviral vector.

29. The modified SIN lentiviral vector of claim 28, wherein the polyA signal sequence comprises a bovine-growth hormone polyA signal sequence.

30. The modified SIN lentiviral vector of claim 28, wherein a U3 region of a 3′-LTR is deleted.

31. The modified SIN lentiviral vector of claim 30, wherein said polyA signal sequence is located at the deleted U3 region, and wherein said polyA signal sequence is derived from an SV40 late polyA signal sequence.

32. The modified SIN lentiviral vector of claim 31, further comprising a chromatin insulator element comprising about 400 base pairs of the distal region of a chicken hypersensitive site-4 (cHS4) element and about 250 base pairs of a core region of a cHS4 element.

33. The modified SIN lentiviral vector of claim 32, wherein the cHS4 element is inside the LTR.

34. The modified SIN lentiviral vector of claim 27, consisting essentially of the packaging signal, the env fragment, the 360 base pair gag fragment, an upstream sequence element (USE) comprising-an SV40 late polyA signal sequence, a bovine growth hormone polyA signal sequence, and one or more chicken hypersensitive site-4 elements (cHS4s).

35. The modified SIN lentiviral vector of claim 27, wherein the transgene of interest is a gamma-globin gene.

36. The modified SIN lentiviral vector of claim 27, wherein the gamma-globin gene is a human gamma-globin gene.

37. The modified SIN lentiviral vector of claim 39, wherein the gamma-globin gene is in a reverse orientation to a viral transcription unit in the lentiviral vector backbone.

38. The modified SIN lentiviral vector of claim 27, wherein the transgene of interest is in operable linkage to a promoter.

39. The modified SIN lentiviral vector of claim 35, wherein the gamma-globin gene comprises a gamma-globin coding sequence and a beta-globin sequence.

40. The modified SIN lentiviral vector of claim 45, wherein the modified SIN lentiviral vector further comprises a beta-globin non-coding and regulatory sequences comprising one or more noncoding region selected from HS2, HS3, and HS4 of the locus control region (LCR) of beta-globin gene.

41. The modified SIN lentiviral vector of claim 27, wherein the gamma-globin gene is under the control of the beta-globin regulatory sequences.

42. A host cell comprising the modified SIN lentiviral vector of claim 27.

43. The host cell of claim 42, wherein the host cell is a hematopoietic stem cell.

44. A method for preparing a transgenic host cell, comprising transfecting a host cell with the modified SIN lentiviral vector of claim 27.

45. The modified SIN lentiviral vector of claim 32, wherein the cHS4 element comprises a core sequence of a cHS4 insulator comprising about 250 base pairs linked to a distal element of the cHS4, comprising about 400 base pairs.
